(19) United States
(12) Patent Application Publication
(10) Pub. No.: US 2026/0209183 A1
(43) Pub. Date: Jul. 23, 2026

(54) HETEROCYCLIC COMPOUNDS AND USES THEREOF

(71) Applicant: CERTA THERAPEUTICS PTY LTD, Melbourne, Victoria (AU)

(72) Inventors: Lorna MITCHELL, Melbourne (AU); Chris BURNS, Melbourne (AU); Jean-Marc GARNIER, Parkville (AU); Jonathan POWELL, Essex (GB); Antonio DORE, Essex (GB); Robert DOWNHAM, Essex (GB)

(21) Appl. No.: 19/138,783

(22) PCT Filed: Nov. 30, 2023
(86) PCT No.: PCT/AU2023/051235

§ 371 Date: Jun. 13, 2025

(30) Foreign Application Priority Data
Dec. 16, 2022 (AU) 2022903880

(51) Int. Cl. C07D 231/56; A61K 31/416; A61K 31/422; A61K 31/4245; A61K 31/4439; A61K 31/506; C07D 401/12; C07D 403/12; C07D 405/04; C07D 405/06; C07D 409/12; C07D 413/06; C07D 413/12

(52) U.S. Cl. CPC C07D 231/56; A61K 31/416; A61K 31/422; A61K 31/4245; A61K 31/4439; A61K 31/506; C07D 401/12; C07D 403/12; C07D 405/04; C07D 405/06; C07D 409/12; C07D 413/06; C07D 413/12

(57) ABSTRACT

The present disclosure relates to compounds useful as modulators of proton-activated G protein-coupled receptors, and pharmaceutical compositions comprising same. The present disclosure also relates to methods of using such compounds and pharmaceutical compositions for the treatment and/or prevention of diseases, disorders or conditions mediated by proton-activated G pro-tein-coupled receptors.

### TECHNICAL FIELD

The present disclosure relates to novel compounds useful as modulators of proton-activated G protein-coupled receptors. The present disclosure further relates to methods of making such compounds, pharmaceutical compositions and medicaments comprising such compounds, and methods of using such compounds to treat, prevent or diagnose diseases, disorders, or conditions associated with proton-activated G protein-coupled receptors.

### BACKGROUND

Local acidification is a common feature of many disease processes such as inflammation, infarction, or solid tumour growth. Acidic pH is not merely a consequence of disease but contributes to recruitment and regulation of immune cells, modifies metabolism of parenchymal, immune and tumour cells, modulates fibrosis, vascular permeability, oxygen availability, and consumption, invasiveness of tumour cells, and impacts on cell survival. Thus, pH-sensing mechanisms are required in cells involved in these processes. These pH sensors play important roles in normal physiology and pathophysiology.

Among the pH-sensing mechanisms, the proton-activated G protein-coupled receptors (GPCRs), in particular GPR68 (OGR1), GPR4 (GPR4), and GPR65 (TDAG8), have emerged as important targets. These receptors are widely expressed, upregulated in inflammation and tumours, sense changes in extracellular pH in the range between pH 8 and 6, and are involved in modulating key pathological processes such as the genesis and progression of several inflammatory diseases (asthma, inflammatory bowel disease), tumour cell metabolism and invasiveness, and fibrosis (Silva et al., Am J Physiol Cell Physiol., 1; 323(2): C400, 2022; Silva and Wagner, Pflugers Arch., 474(5): 487, 2022.).

There is a need for new therapies to ameliorate, treat and/or prevent diseases, disorders, or conditions associated with proton-activated GPCRs, such as inflammatory, proliferative or fibrotic disorders, or cancer.

### SUMMARY

The present inventors have identified compounds effective for modulating, such as inhibiting, proton-activated G protein-coupled receptors, such as GPR68 (OGR1). The identified compounds are useful in the treatment, prevention and/or diagnosis of diseases, disorders, or conditions associated with proton-activated G protein-coupled receptors, such as inflammatory, fibrotic, or proliferative diseases, disorders or conditions, and/or cancer.

Accordingly, in one aspect of the present disclosure, there is provided a compound having the structure of Formula (II):

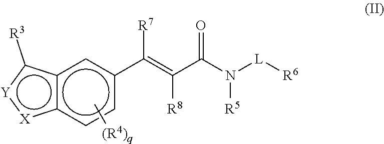

or a pharmaceutically acceptable salt, solvate or stereoisomer thereof,
wherein:
X is selected from N and NR¹;
Y is selected from N and NR²;
R¹ and R² are independently selected from H, C₁₋₁₀ alkyl, C₁₋₁₀ heteroalkyl, C₂₋₁₀ alkenyl, C₂₋₁₀ alkynyl, C₃₋₆ carbocyclyl, heterocyclyl, aryl, and heteroaryl, each of which is optionally substituted with one or more substituents selected from C₁₋₁₀ alkyl, halogen, —CN, —OR⁹, —SR⁹, —S(O)₂R⁹, —NR⁹R¹⁰, —NO₂, ═(O), ═(S), —(N)R⁹, ═(N)R¹⁰, —C(O)R⁹, —C(O)NR⁹R¹⁰, —C(O)SR⁹ or —C(O)OR⁹; or with one or more substituents selected from C₃₋₆ carbocyclyl, heterocyclyl, aryl, and heteroaryl, each of which is optionally substituted with one or more substituents selected from C₁₋₁₀ alkyl, halogen, —CN, —OR⁹, —SR⁹, —S(O)₂R⁹, —NR⁹R¹⁰, —NO₂, ═(O), ═(S), ═(N)R⁹, ═(N)R¹⁰, —C(O)R⁹, —C(O)NR⁹R¹⁰, —C(O)SR⁹ or —C(O)OR⁹;
or R¹ and R², together with the atoms to which they are attached, form a fused 5-6 membered heteroaryl or a fused 5-6 membered heterocyclyl, each of which is optionally substituted with one or more substituents selected from optionally substituted C₁₋₁₀ alkyl, halogen, —CN, —OR⁹, —SR⁹, —S(O)₂R⁹, —NR⁹R¹⁰, —NO₂, ═(O), ═(S), ═(N)R⁹, —(N)R¹⁰, —C(O)R⁹, —C(O)NR⁹R¹⁰, —C(O)SR⁹ or —C(O)OR⁹;
each R⁹ is independently selected from H, optionally substituted C₁₋₆ alkyl, and optionally substituted C₅₋₆ aryl;
each R¹⁰ is independently selected from H, optionally substituted C₁₋₆ alkyl, optionally substituted C₅₋₆ aryl, and —C(O)R⁹;
R³ is selected from H, halogen, hydroxy, optionally substituted C₁₋₆ alkyl, optionally substituted C₂₋₆ alkenyl, optionally substituted C₂₋₆ alkynyl, optionally substituted C₁₋₆ alkylthio, optionally substituted C₂₋₈ alkoxyalkyl, optionally substituted C₃₋₁₀ carbocyclyl, optionally substituted C₅₋₁₀ aryl, —CN, —OR⁹, —SR⁹, —NR⁹R¹⁰, —C(O)R⁹, —C(O)OR⁹, —S(O)₂R⁹, and —NO₂;
or R³ and R², together with the atoms to which they are attached, form a fused 5-6 membered heteroaryl or a fused 5-6 membered heterocyclyl, each of which is optionally substituted with one or more substituents selected from C₁₋₁₀ alkyl, halogen, —CN, —OR⁹, —SR⁹, —S(O)₂R⁹, —NR⁹R¹⁰, —NO₂, —(O), —(S), —(N)R⁹, ═(N)R¹⁰, —C(O)R⁹, —C(O)NR⁹R¹⁰, —C(O)SR⁹ or —C(O)OR⁹;
q is an integer from 0 to 3;
R⁴ is selected from halogen, hydroxy, optionally substituted C₁₋₆ alkyl, optionally substituted C₂₋₆ alkenyl, optionally substituted C₂₋₆ alkynyl, optionally substituted C₁₋₆ alkylthio, optionally substituted C₂₋₈ alkoxyalkyl, optionally substituted C₃₋₁₀ carbocyclyl, optionally substituted C₆₋₁₀ aryl, —CN, —OR⁹, —SR⁹, —NR⁹R¹⁰, —C(O)R⁹, —C(O)OR⁹, —S(O)₂R⁹, and —NO₂;
R⁵ is selected from H, optionally substituted C₁₋₆ alkyl, optionally substituted C₂₋₆ alkenyl, optionally substituted C₂₋₆ alkynyl, optionally substituted C₂₋₈ alkoxyalkyl, optionally substituted C₃₋₆ carbocyclyl, optionally substituted C₅₋₁₀ aryl, optionally substituted 5-6 membered heteroaryl, and optionally substituted 5-6 membered heterocyclyl;
L is a bond, or a linker selected from —C(O)N(R⁹)—, —S(O)₂—, —C(O)—, or —C(O)O—; or from optionally substituted C₁₋₄ alkylene, optionally substituted C₂₋₄ alkenylene, —OC₁₋₄ alkylene, or —OC₂₋₄ alkenylene, each of which is optionally interrupted with —O—, —S—, —NR⁹—, —NR¹⁰—, —C(O)N(R⁹)—, —N(R⁹) C(O)—, —S(O)₂—, —N(R⁹) S(O)₂—, —S(O)₂N(R⁹)—, —C(O)—, —C(O)O—, or —O(O)C—;
R⁶ is selected from C₅₋₁₀ aryl, 5-10 membered heteroaryl, 3-10 membered heterocyclyl, C3-10 carbocyclyl, C₁₋₁₀ alkyl, C₂₋₁₀ alkenyl, or C₂₋₁₀ alkynyl, each of which is optionally substituted with one or more substituents optionally selected from optionally substituted C₁₋₆ alkyl, optionally substituted C₅₋₆ aryl, optionally substituted 5-6 membered heteroaryl, optionally substituted 5-6 membered heterocyclyl, halogen, —CN, —OR¹¹, —SR¹¹, —S(O)₂R¹¹, —S(O)₂N(R¹¹)₂, —S(O)(NR¹⁰)R¹¹, —C(O)N(R¹¹)₂, —NR¹¹C(O)R¹¹, —N(R¹¹) S(O)₂R¹¹, —N(R¹¹)₂, —NO₂, ═(O), ═(S), ═(N)R¹¹, —C(O)R¹¹, —C(O)OR¹¹, —C(O)N(R¹¹)₂, or —C(O)SR¹¹; wherein two adjacent optional substituents, when present, together with the atoms to which they are attached, may form an optionally substituted fused 5-6 membered heteroaryl or an optionally substituted fused 5-6 membered heterocyclyl;
or R⁶ and R⁵, together with the atoms to which they are attached, form a 3-10 membered heterocyclyl or 5-10 membered heteroaryl, each of which is optionally substituted with one or more substituents optionally selected from optionally substituted C₁₋₁₀ alkyl, optionally substituted C₂₋₆ alkenyl, optionally substituted C₂₋₆ alkynyl, halogen, —CN, —OR¹¹, —SR¹¹, —S(O)₂R¹¹, —S(O)₂N(R¹¹)₂, —S(O)(NR¹⁰)R¹¹, —C(O) N(R¹¹)₂, —NR¹¹C(O)R¹¹, —N(R¹¹) S(O)₂R¹¹, —N(R¹¹)₂, —NO₂, ═(O),═(S), ═(N)R¹¹, —C(O)R¹¹, —C(O)OR¹¹, —C(O)N(R¹¹)₂, or —C(O)SR¹¹;
each R¹¹ is independently selected from H, optionally substituted C₁₋₆ alkyl, optionally substituted C₂₋₆ alkenyl, optionally substituted C₂₋₆ alkynyl, optionally substituted C₅₋₁₀ aryl, optionally substituted C₂₋₂₀ alkoxyalkyl, C₁-C₆ haloalkyl, —C(O)R⁹, —OR⁹, —S(O)₂R⁹, and —S(O)₂N(R⁹)₂.
R⁷ and R⁸ are independently selected from H, C₁₋₆ alkyl, C₅₋₁₀ aryl, 5-10 membered heteroaryl, and C₃₋₁₀ carbocyclyl, each of which is optionally substituted with one or more substituents selected from C₁₋₆ alkyl, C₂₋₆ alkenyl, C₂₋₆ alkynyl, C₅₋₆ aryl, halogen, —CN, —OR⁹, —SR⁹, —S(O)₂R⁹, —NR⁹R¹⁰, —NO₂, —(O), ═(S), ═(N)R⁹, ═(N)R¹⁰, —C(O)R⁹, —C(O)NR⁹R¹⁰, —C(O)SR⁹ or —C(O)OR⁹.

In some embodiments, the compound as defined herein is having the structure of Formula (III):

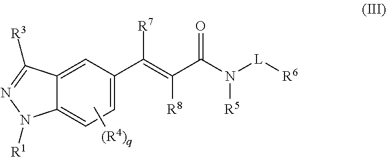

or a pharmaceutically acceptable salt, solvate or stereoisomer thereof.

In some embodiments, the compound as defined herein is having the structure of Formula (IV):

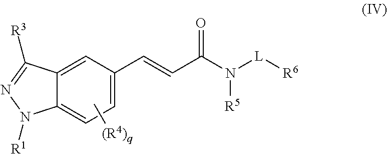

or a pharmaceutically acceptable salt, solvate or stereoisomer thereof.

In some embodiments, R¹ is C₁₋₁₀ alkyl, C₂₋₁₀ alkenyl, C₂₋₁₀ alkynyl, or C₃₋₆ carbocyclyl, each of which is optionally substituted with one or more substituents selected from C₁₋₆ alkyl, halogen, —CN, OR⁹, and —SR⁹; or with one or more groups selected from C₃₋₆ carbocyclyl, 5-6 membered heterocyclyl, C₅₋₆ aryl, and 5-6 membered heteroaryl, each of which is optionally substituted with one or more substituents selected from C₁₋₆ alkyl, halogen, —CN, —OR⁹, —SR⁹, —S(O)₂R⁹, —NR⁹R¹⁰, —NO₂, ═(O),═(S), ═(N)R⁹, ═(N)R¹⁰, —C(O)R⁹, —C(O)NR⁹R¹⁰, —C(O)SR⁹ or —C(O)OR⁹.

In some embodiments, R¹ is C₁₋₆ alkyl, optionally substituted with one or more substituents selected from C₁₋₆ alkyl, halogen, —CN, —OR⁹, and —SR⁹; or with one or more groups selected from C₃₋₆ carbocyclyl, 5-6 membered heterocyclyl, C₅₋₆ aryl, and 5-6 membered heteroaryl, each of which is optionally substituted with one or more substituents selected from C₁₋₆ alkyl, halogen, —CN, —OR⁹, —SR⁹, —S(O)₂R⁹, —NR⁹R¹⁰, —NO₂,═(O),═(S), ═(N)R⁹, ═(N)R¹⁰, —C(O)R⁹, —C(O)NR⁹R¹⁰, —C(O)SR⁹ or —C(O)OR⁹.

In some embodiments, R¹ is C₁₋₆ alkyl, optionally selected from methyl, ethyl, propyl or isopropyl.

In some embodiments, R¹ is C₁₋₆ alkyl substituted with halogen, —CN, —OR⁹, —C(O)R⁹, —C(O)NR⁹R¹⁰, C(O)SR⁹ or —C(O)OR⁹, optionally wherein R⁹ is methyl.

In some embodiments, R¹ is C₁₋₆ alkyl substituted with optionally substituted 5-6 membered heteroaryl or optionally substituted 3-6 membered heterocyclyl.

In some embodiments, R¹ is C₁₋₄ alkyl substituted with a 3-6 membered heterocyclyl selected from:

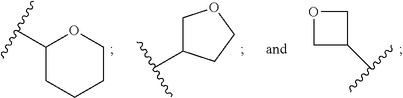 [SMILES: *C1CCCCO1 ; *C1CCOC1 ; *C1COC1]

each of which may be optionally substituted.

In some embodiments, R¹ is C₁₋₆ alkyl substituted with optionally substituted C₁₋₆ carbocyclyl.

In some embodiments, R¹ is C₁₋₄ alkyl substituted with C₁₋₆ carbocyclyl, optionally wherein C₁₋₆ carbocyclyl is selected from optionally substituted cyclobutyl or optionally substituted cyclopropyl.

In some embodiments, R¹ is

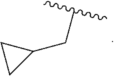 [SMILES: *CC1CC1]

In some embodiments, R³ is H.

In some embodiments, q is 0 or 1.

In some embodiments, the compound as defined herein is having the structure of Formula (Va), (Vb) or (Vc):

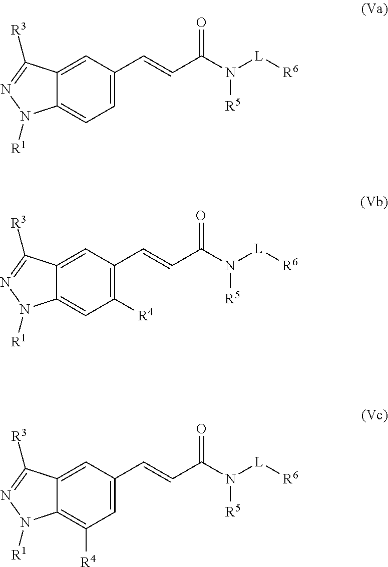

or a pharmaceutically acceptable salt, solvate or stereoisomer thereof.

In some embodiments, R⁴ is selected from halogen, hydroxy, optionally substituted C₂₋₈ alkoxyalkyl, —CN, —OR⁹, —SR⁹, —NR′R¹⁰, —C(O)R⁹, —C(O)OR⁹, —S(O)₂R⁹, and —NO₂.

In some embodiments, R⁴ is selected from halogen, —CN, and —NO₂, optionally wherein halogen is F.

In some embodiments, the compound as defined herein is having the structure of Formula (VI):

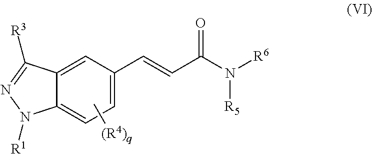

or a pharmaceutically acceptable salt, solvate or stereoisomer thereof.

In some embodiments, the compound as defined herein is having the structure of Formula (VIa), (VIb) or (VIc):

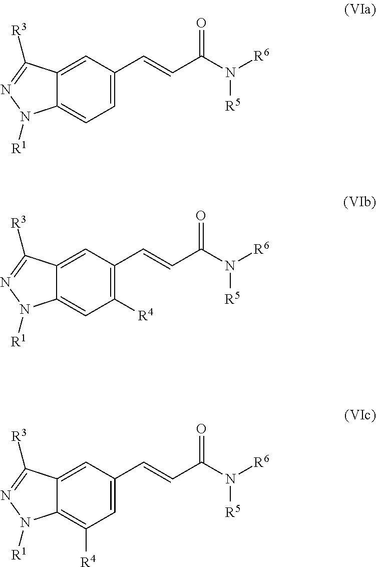

or a pharmaceutically acceptable salt, solvate or stereoisomer thereof.

In some embodiments, R⁵ is H, optionally substituted C₁₋₄ alkyl, optionally substituted C₂₋₄ alkenyl, optionally substituted C₂₋₄ alkynyl, or optionally substituted C₂₋₈ alkoxyalkyl.

In some embodiments, R⁵ is H.

In some embodiments, R⁶ is selected from C₅₋₁₀ aryl, 5-10 membered heteroaryl, 3-10 membered heterocyclyl, C₃₋₁₀ carbocyclyl, C₁₋₁₀ alkyl, C₂₋₁₀ alkenyl, or C₂₋₁₀ alkynyl, each of which is optionally substituted with one or more substituents optionally selected from optionally substituted C₁₋₆ alkyl, optionally substituted C₅₋₆ aryl, optionally substituted 5-6 membered heteroaryl, optionally substituted 5-6 membered heterocyclyl, halogen, —CN, —OR¹¹, —SR¹¹, —S(O)₂R¹¹, —S(O)₂N(R¹¹)₂, —S(O)(NR¹⁰)R¹¹, —C(O)N(R¹¹)₂, —NR¹¹C(O)R¹¹, —N(R¹¹) S(O)₂R¹¹, —N(R¹¹)₂, —NO₂, ═(O),═(S), ═(N)R¹¹, —C(O)R¹¹, —C(O)OR¹¹, —C(O)N(R¹¹)₂, or —C(O)SR¹¹; wherein two adjacent optional substituents, when present, together with the atoms to which they are attached, may form an optionally substituted fused 5-6 membered heteroaryl or an optionally substituted fused 5-6 membered heterocyclyl.

In some embodiments, R⁶ is selected from optionally substituted C6 aryl, optionally substituted 5 membered heteroaryl, and optionally substituted 6 membered heteroaryl.

In some embodiments, R⁶ is selected from:

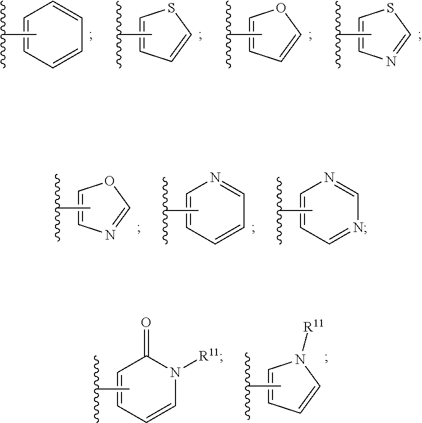

each of which may be optionally substituted.

In some embodiments, R⁶ is selected from:

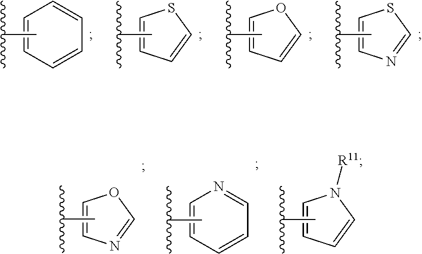

each of which may be optionally substituted.

In some embodiments, the optional substituent of R⁶ is one or more substituents selected from halogen, optionally substituted 5 membered heterocyclyl, optionally substituted 5 membered heteroaryl, —C(O)OR¹¹, —N(R¹¹) S(O)₂R¹¹, —C(O) N(R¹¹)₂, and —S(O)₂N(R¹¹) 2.

In some embodiments, the compound as defined herein is selected from the group consisting of:

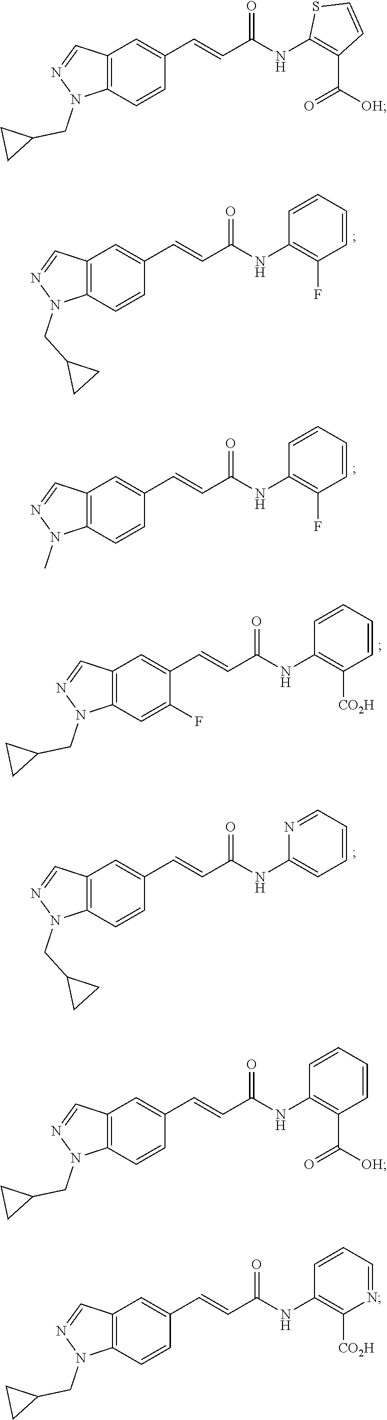

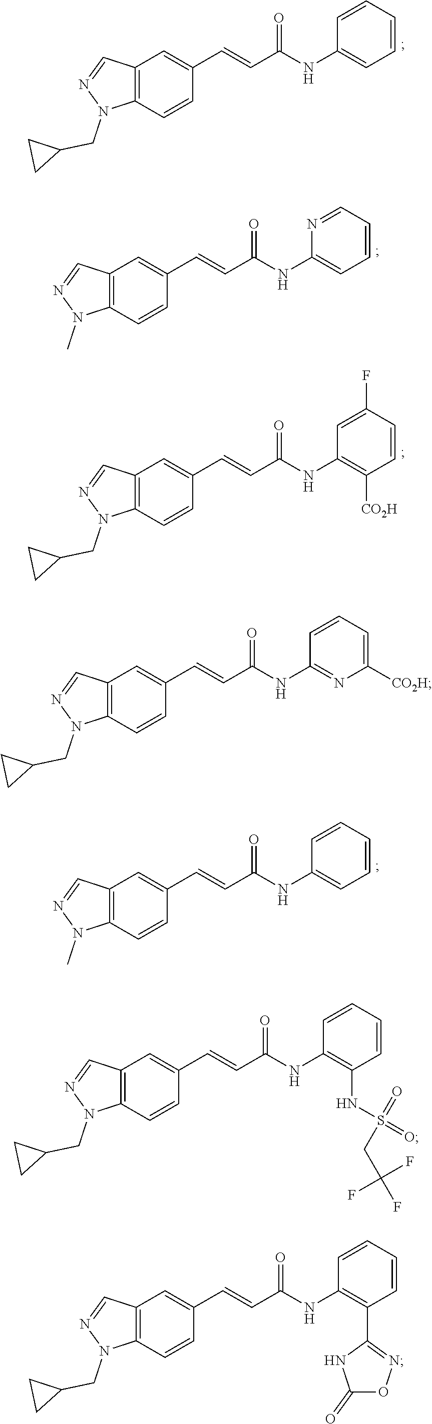

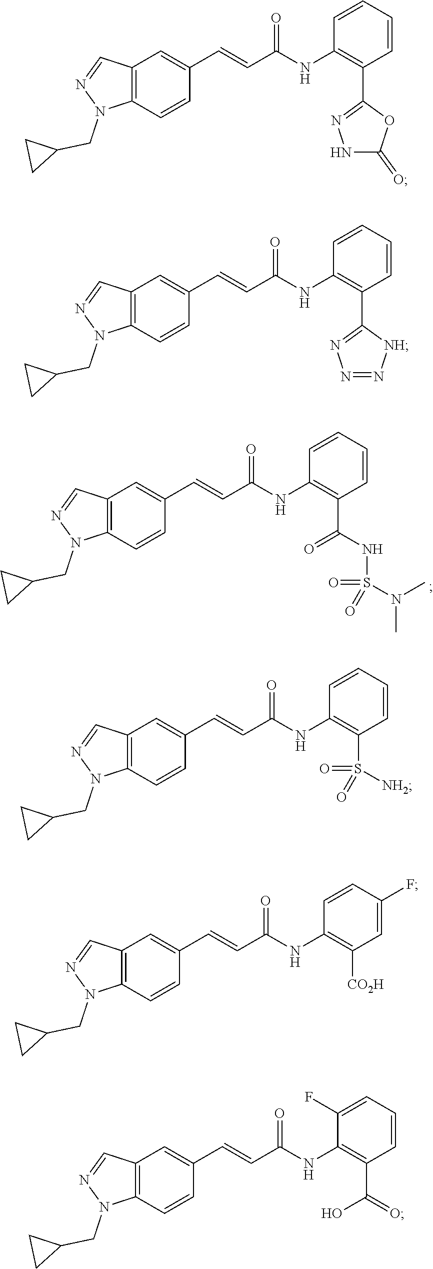 [SMILES: O=C(/C=C/c1ccc2c(cnn2CC2CC2)c1)Nc1ccccc1-c1n[nH]c(=O)o1 ; O=C(/C=C/c1ccc2c(cnn2CC2CC2)c1)Nc1ccccc1-c1nnn[nH]1 ; CN(C)S(=O)(=O)NC(=O)c1ccccc1NC(=O)/C=C/c1ccc2c(cnn2CC2CC2)c1 ; NS(=O)(=O)c1ccccc1NC(=O)/C=C/c1ccc2c(cnn2CC2CC2)c1 ; O=C(/C=C/c1ccc2c(cnn2CC2CC2)c1)Nc1ccc(F)cc1C(=O)O ; O=C(/C=C/c1ccc2c(cnn2CC2CC2)c1)Nc1c(F)cccc1C(=O)O]

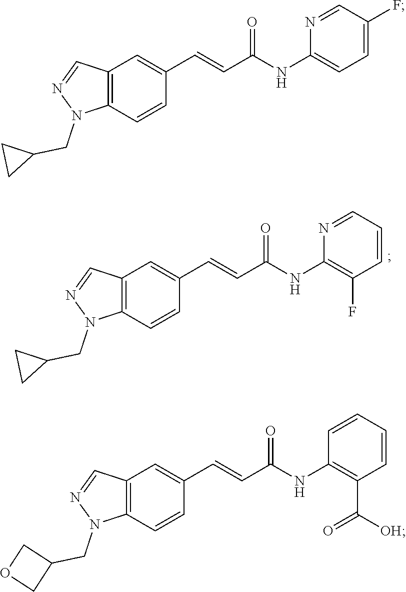 [SMILES: O=C(/C=C/c1ccc2c(cnn2CC2CC2)c1)Nc1ccc(F)cn1 ; O=C(/C=C/c1ccc2c(cnn2CC2CC2)c1)Nc1ncccc1F ; O=C(/C=C/c1ccc2c(cnn2CC2COC2)c1)Nc1ccccc1C(=O)O]

or a pharmaceutically acceptable salt, solvate or stereoisomer thereof.

In some embodiments, the compound as defined herein is selected from the group consisting of:

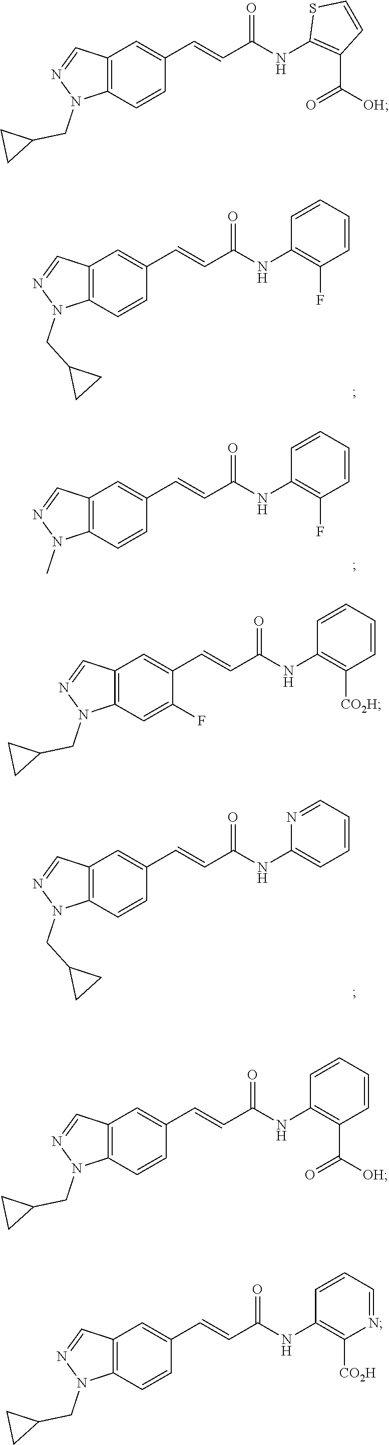

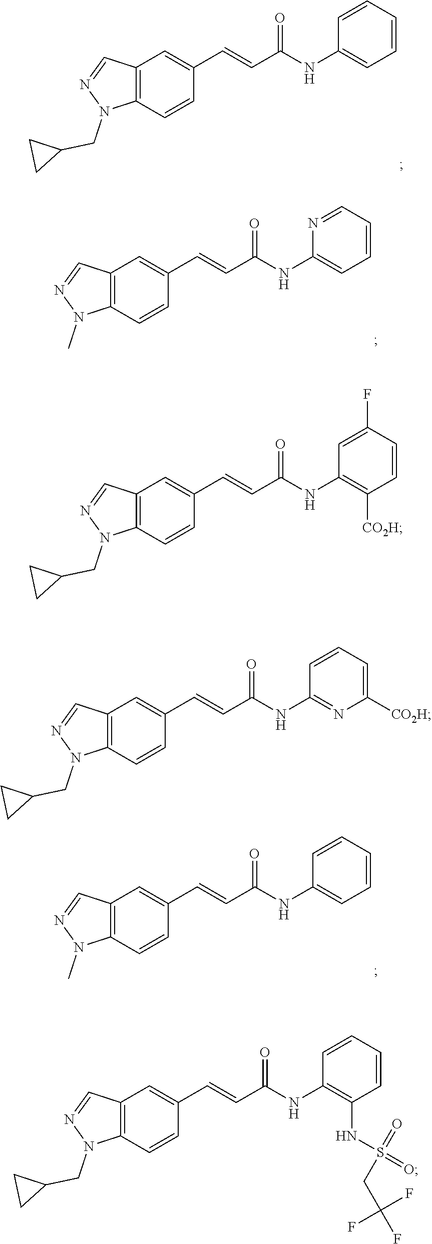 [SMILES: O=C(/C=C/c1ccc2c(cnn2CC2CC2)c1)Nc1ccccc1 ; Cn1ncc2cc(/C=C/C(=O)Nc3ccccn3)ccc21 ; O=C(/C=C/c1ccc2c(cnn2CC2CC2)c1)Nc1cc(F)ccc1C(=O)O ; O=C(/C=C/c1ccc2c(cnn2CC2CC2)c1)Nc1cccc(C(=O)O)n1 ; Cn1ncc2cc(/C=C/C(=O)Nc3ccccc3)ccc21 ; O=C(/C=C/c1ccc2c(cnn2CC2CC2)c1)Nc1ccccc1NS(=O)(=O)CC(F)(F)F]

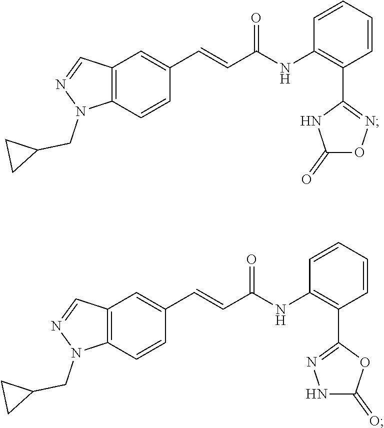 [SMILES: O=C(/C=C/c1ccc2c(cnn2CC2CC2)c1)Nc1ccccc1-c1noc(=O)[nH]1 ; O=C(/C=C/c1ccc2c(cnn2CC2CC2)c1)Nc1ccccc1-c1n[nH]c(=O)o1]

or a pharmaceutically acceptable salt, solvate or stereoisomer thereof.

In some embodiments, the compound as defined herein is selected from the group consisting of:

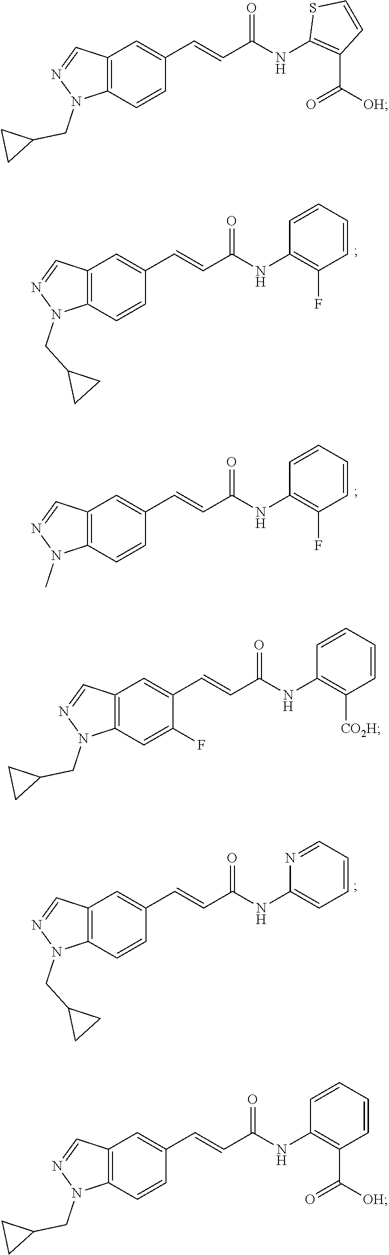 [SMILES: O=C(/C=C/c1ccc2c(cnn2CC2CC2)c1)Nc1sccc1C(=O)O ; O=C(/C=C/c1ccc2c(cnn2CC2CC2)c1)Nc1ccccc1F ; Cn1ncc2cc(/C=C/C(=O)Nc3ccccc3F)ccc21 ; O=C(/C=C/c1cc2cnn(CC3CC3)c2cc1F)Nc1ccccc1C(=O)O ; O=C(/C=C/c1ccc2c(cnn2CC2CC2)c1)Nc1ccccn1 ; O=C(/C=C/c1ccc2c(cnn2CC2CC2)c1)Nc1ccccc1C(=O)O]

or a pharmaceutically acceptable salt, solvate or stereoisomer thereof.

In some embodiments, R⁶ is selected from optionally substituted 3-10 membered heterocyclyl and optionally substituted C₃₋₁₀ carbocyclyl.

In some embodiments, the optional substituent of R⁶ is one or more substituents selected from halogen and —C(O)OR¹¹, optionally wherein R¹¹ is H or optionally substituted C₁₋₆ alkyl.

In some embodiments, the compound as defined herein is selected from the group consisting of:

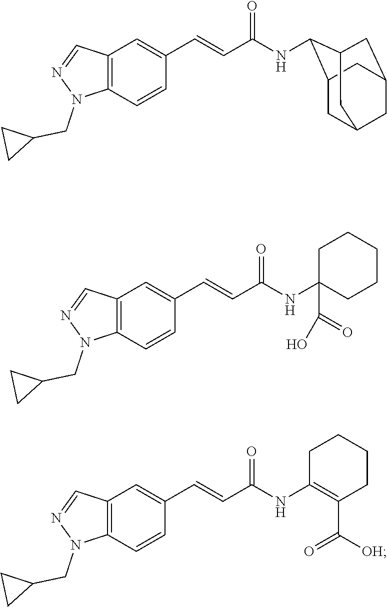 [SMILES: O=C(/C=C/c1ccc2c(cnn2CC2CC2)c1)NC1C2CC3CC(C2)CC1C3 ; O=C(/C=C/c1ccc2c(cnn2CC2CC2)c1)NC1(C(=O)O)CCCCC1 ; O=C(/C=C/c1ccc2c(cnn2CC2CC2)c1)NC1=C(C(=O)O)CCCC1]

or a pharmaceutically acceptable salt, solvate or stereoisomer thereof.

In some embodiments, R⁶ and R⁵, together with the atoms to which they are attached, form a 3-10 membered heterocyclyl or 5-10 membered heteroaryl, each of which is optionally substituted with one or more substituents optionally selected from optionally substituted C₁₋₁₀ alkyl, optionally substituted C₂₋₆ alkenyl, optionally substituted C₂₋₆ alkynyl, halogen, —CN, —OR¹¹, —SR¹¹, —S(O)₂R¹¹, —S(O)₂N(R¹¹)₂, —S(O)(NR¹⁰)R¹¹, —C(O)N(R¹¹)₂, —NR¹¹C(O)R¹¹, —N(R¹¹) S(O)₂R¹¹, —N(R¹¹)₂, —NO₂, ═(O), ═(S), ═(N)R¹¹, —C(O)R¹¹, —C(O)OR¹¹, —C(O)N(R¹¹)₂, or —C(O)SR¹¹.

In some embodiments, the optional substituent of R⁶ is one or more substituents selected from halogen and —C(O)OR¹¹, optionally wherein R¹¹ is H or optionally substituted C₁₋₆ alkyl.

In some embodiments, the compound as defined herein is having the structure:

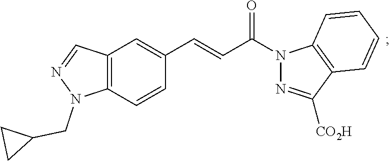 [SMILES: O=C(O)c1nn(C(=O)/C=C/c2ccc3c(cnn3CC3CC3)c2)c2ccccc12]

or a pharmaceutically acceptable salt, solvate or stereoisomer thereof.

There is also provided a compound having the following structure:

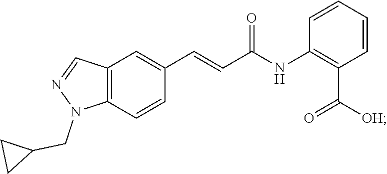 [SMILES: O=C(/C=C/c1ccc2c(cnn2CC2CC2)c1)Nc1ccccc1C(=O)O]

or a pharmaceutically acceptable salt thereof.

In some embodiments, the compound or pharmaceutically acceptable salt thereof is

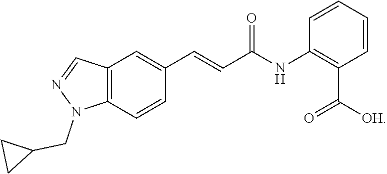 [SMILES: O=C(/C=C/c1ccc2c(cnn2CC2CC2)c1)Nc1ccccc1C(=O)O]

There is also provided a pharmaceutical composition comprising a compound as defined herein, or a pharmaceutically acceptable salt, solvate or stereoisomer thereof, and a pharmaceutically acceptable excipient.

There is also provided a method of treating and/or preventing a disease, disorder or condition mediated by a proton-activated GPCR in a subject in need thereof, comprising administering to the subject an effective amount of a compound as defined herein, or a pharmaceutically acceptable salt, solvate or stereoisomer thereof, or a pharmaceutical composition as defined herein.

There is also provided a use of a compound as defined herein, or a pharmaceutically acceptable salt, solvate or stereoisomer thereof, or a pharmaceutical composition as defined herein, in the manufacture of a medicament for the treatment and/or prevention of a disease, disorder or condition mediated by a proton-activated GPCR in a subject in need thereof.

There is also provided a compound as defined herein, or a pharmaceutically acceptable salt, solvate or stereoisomer thereof, or a pharmaceutical composition as defined herein, for use in the treatment and/or prevention of a disease, disorder or condition mediated by a proton-activated GPCR in a subject in need thereof.

In some embodiments, the proton-activated GPCR is a GPR68 (OGR1).

In some embodiments, the disease, disorder or condition is selected from the group consisting of: an inflammatory disease, disorder or condition; a fibrotic disease, disorder or condition; a proliferative disease, disorder or condition, or a cancer.

In some embodiments, the disease, disorder or condition is an inflammatory disease, disorder or condition. In some embodiments, the inflammatory disease, disorder or condition is selected from focal segmental glomerulosclerosis, lupus nephritis, membranous nephropathy, IgA nephropathy, chronic obstructive pulmonary disorder (COPD), asthma, and cystic fibrosis.

In some embodiments, the inflammatory disease, disorder or condition is an autoimmune disease, disorder or condition. In some embodiments, the autoimmune disease, disorder or condition is selected from systemic lupus erythematosus (SLE), inflammatory bowel disease, Crohn's disease, ulcerative colitis, graft versus host disease, multiple sclerosis, and rheumatoid arthritis.

In some embodiments, the disease, disorder or condition is a fibrotic disease, disorder or condition. In some embodiments, the fibrotic disease, disorder or condition is selected from renal fibrosis, pulmonary fibrosis, cardiovascular fibrosis, ocular fibrosis, dermal fibrosis, pancreatic fibrosis, and liver fibrosis. In some embodiments, the fibrotic disease, disorder or condition is selected from diabetic cardiomyopathy, congestive heart failure, ischemic heart disease, hypertension, peripheral artery disease, cerebrovascular disease, chronic kidney disease, diabetic nephropathy, systemic sclerosis (scleroderma), hypertrophic scars, keloids, idiopathic pulmonary fibrosis (IPF), non-alcoholic steatohepatitis (NASH), non-alcoholic fatty liver disease (NAFLD), primary biliary cirrhosis (PBC), and primary sclerosis cholangitis (PSC).

In some embodiments, the disease, disorder or condition is a proliferative disease, disorder or condition. In some embodiments, the proliferative disease, disorder or condition is selected from diabetic retinopathy, diabetic macular edema, macular degeneration, retinopathy of prematurity (ROP), and proliferative vitreoretinopathy (PVR).

In some embodiments, the disease, disorder or condition is a cancer. In some embodiments, the cancer is selected from a breast cancer, skin cancer (melanoma), pancreatic cancer, lung cancer, prostate cancer, colon cancer, liver cancer (hepatocellular carcinoma), head and neck cancer, ovarian cancer and kidney cancer.

In some embodiments, the subject is a human.

There is also provided a method of inhibiting a proton-activated GPCR in a cell, comprising administering to the cell an effective amount of a compound as defined herein, or a pharmaceutically acceptable salt, solvate or stereoisomer thereof, or a pharmaceutical composition as defined herein.

There is also provided a use of a compound as defined herein, or a pharmaceutically acceptable salt, solvate or stereoisomer thereof, or a pharmaceutical composition as defined herein, in the manufacture of a medicament for the inhibition of a proton-activated GPCR in a cell.

There is also provided a compound as defined herein, or a pharmaceutically acceptable salt, solvate or stereoisomer thereof, or a pharmaceutical composition as defined herein, for use in the inhibition of a proton-activated GPCR in a cell.

In some embodiments, the proton-activated GPCR is a GPR68 (OGR1).

In some embodiments, the cell is a tumour cell.

### DETAILED DESCRIPTION

Unless specifically defined otherwise, all technical and scientific terms used herein shall be taken to have the same meaning as commonly understood by one of ordinary skill in the art (e.g., chemistry, biochemistry, cell culture, molecular biology and pharmacy).

All patents, applications, published applications and other publications referenced herein are incorporated by reference in their entirety unless stated otherwise.

Unless otherwise indicated, conventional methods of mass spectroscopy, NMR, HPLC, protein chemistry, biochemistry, recombinant DNA techniques and pharmacology are employed.

Unless otherwise required by context, singular terms shall include pluralities and plural terms shall include the singular. Thus, as used in this specification and the appended claims, the singular forms “a”, “an” and “the” include plural referents unless the context clearly indicates otherwise. For example, the term “an subject” means “one or more subjects” unless the context clearly indicates otherwise.

The term “about” as used herein refers to a range of +/−5% of the specified value.

Throughout this specification, various aspects and components of the disclosure can be presented in a range format. The range format is included for convenience and should not be interpreted as an inflexible limitation on the scope of the disclosure. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 5, 5.5 and 6, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps. Furthermore, use of the term “including” as well as other forms, such as “include”, “includes,” and “included,” is not limiting.

The use of “or” or “and” means “and/or” unless stated otherwise.

The compounds of the present disclosure can exist as solvates. The term “solvate” refers to the compound formed by the interaction of a solvent and a compound described herein or salt thereof. Suitable solvates are pharmaceutically acceptable solvates including hydrates.

The compounds of the present disclosure can exist as pharmaceutically acceptable salts. The term “pharmaceutically acceptable salt” refers to salts that retain the biological effectiveness and properties of a compound and which are not biologically or otherwise undesirable for use in a pharmaceutical. In many cases, the compounds disclosed herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)—and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and tert-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the group alkyl as defined above which contains at least one O atom, where the at least one oxygen atom is at the position where the alkoxy group is attached to the remainder of the compound. By way of non-limiting example, suitable alkoxy groups include, e.g., methoxy (—O—CH₃), ethoxy (—O—CH₂—CH₃), propoxy (—O—CH₂—CH₂—CH₃ (straight chain alkyl) or —O—CH—(CH₃)₂ (branched chain alkyl)) and —O—CH₂—CH₂—O—CH₃. The alkoxy may be substituted or unsubstituted. The alkoxy may be substituted or unsubstituted. In this regard, the term “haloalkoxy”, as used herein, refers to “alkoxy” substituted with one or more halo i.e. one or more of F, Cl, Br or I. An example of “haloalkoxy” is —OCF₃.

As used herein, “alkylthio” refers to the formula-SR wherein R is an alkyl as is defined above, such as “C₁₋₉ alkylthio” and the like, including but not limited to methylmercapto, ethylmercapto, n-propylmercapto, 1-methylethylmercapto (isopropylmercapto), n-butylmercapto, iso-butylmercapto, sec-butylmercapto, tert-butylmercapto, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2, -dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atom, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. The heteroalkyl group may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

As used herein, “alkylene” means a branched, or straight chain fully saturated di-radical chemical group containing only carbon and hydrogen that is attached to the rest of the molecule via two points of attachment (i.e., an alkanediyl). The alkylene group may have 1 to 20 carbon atoms, although the present definition also covers the occurrence of the term alkylene where no numerical range is designated. The alkylene group may also be a medium size alkylene having 1 to 9 carbon atoms. The alkylene group could also be a lower alkylene having 1 to 4 carbon atoms. The alkylene group may be designated as “C₁₋₄ alkylene” or similar designations. By way of example only, “C₁₋₄ alkylene” indicates that there are one to four carbon atoms in the alkylene chain, i.e., the alkylene chain is selected from the group consisting of methylene, ethylene, ethan-1,1-diyl, propylene, propan-1,1-diyl, propan-2,2-diyl, 1-methyl-ethylene, butylene, butan-1,1-diyl, butan-2,2-diyl, 2-methyl-propan-1,1-diyl, 1-methyl-propylene, 2-methyl-propylene, 1,1-dimethyl-ethylene, 1,2-dimethyl-ethylene, and 1-ethyl-ethylene.

As used herein, “alkenylene” means a straight or branched chain di-radical chemical group containing only carbon and hydrogen and containing at least one carbon-carbon double bond that is attached to the rest of the molecule via two points of attachment. The alkenylene group may have 2 to 20 carbon atoms, although the present definition also covers the occurrence of the term alkenylene where no numerical range is designated. The alkenylene group may also be a medium size alkenylene having 2 to 9 carbon atoms. The alkenylene group could also be a lower alkenylene having 2 to 4 carbon atoms. The alkenylene group may be designated as “C₂₋₄ alkenylene” or similar designations. By way of example only, “C₂₋₄ alkenylene” indicates that there are two to four carbon atoms in the alkenylene chain, i.e., the alkenylene chain is selected from the group consisting of ethenylene, ethen-1,1-diyl, propenylene, propen-1,1-diyl, prop-2-en-1,1-diyl, 1-methyl-ethenylene, but-1-enylene, but-2-enylene, but-1,3-dienylene, buten-1,1-diyl, but-1,3-dien-1,1-diyl, but-2-en-1, 1-diyl, but-3-en-1, 1-diyl, 1-methyl-prop-2-en-1, 1-diyl, 2-methyl-prop-2-en-1,1-diyl, 1-ethyl-ethenylene, 1,2-dimethyl-ethenylene, 1-methyl-propenylene, 2-methyl-propenylene, 3-methyl-propenylene, 2-methyl-propen-1,1-diyl, and 2,2-dimethyl-ethen-1,1-diyl.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A heteroaryl group may, for example, be monocyclic or polycyclic (e.g. bicyclic). A polycyclic heteroaryl may for example contain fused rings. In a bicyclic heteroaryl group there may be one or more heteroatoms in each ring, or heteroatoms only in one of the rings. Examples of monocyclic heteroaryl groups include furanyl, thienyl, pyrrolyl, oxazolyl, thiazolyl, imidazolyl, oxadiazolyl, thiadiazolyl, pyridyl, triazolyl, triazinyl, pyridazyl, isothiazolyl, isoxazolyl, pyrazinyl, pyrazolyl, and pyrimidinyl. Examples of bicyclic heteroaryl groups include quinoxalinyl, quinazolinul, pyridopyrazinyl, benzoxazolyl, benzothiophenyl, benzimidazolyl, naphthyridinyl, quinolinyl, benzofuranyl, indolyl, indazolyl, benzothiazolyl, oxazolyl [4,5-b]pyridyl, pyridopyrimidinyl, isoquinolinyl, and benzohydroxazole.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C1-4 alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle [2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A heterocyclyl group may, for example, be monocyclic or polycyclic (e.g. bicyclic). A polycyclic heterocyclyl may for example contain fused rings. In a bicyclic heterocyclyl group there may be one or more heteroatoms in each ring, or heteroatoms only in one of the rings. Heterocyclyl groups containing a suitable nitrogen atom include the corresponding N-oxides. In one example, the heterocyclyl group is of three to ten atoms (i.e. 3-10 membered heterocyclyl). Examples of monocyclic non-aromatic heterocyclyl groups include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl. Examples of bicyclic heterocyclyl groups in which one of the rings is non-aromatic include dihydrobenzofuranyl, indanyl, indolinyl, isoindolinyl, tetrahydroisoquinolinyl, tetrahydroquinolyl, and benzoazepanyl.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(O)R, wherein R is hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. Non-limiting examples include formyl, acetyl, propanoyl, benzoyl, and acryl.

An “O-carboxy” group refers to a “—OC(O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-carboxy” group refers to a “—C(O)OR” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A non-limiting example includes carboxyl (i.e., —C(O)OH).

A “cyano” group refers to a “—CN” group. A “cyanato” group refers to an “—OCN” group. An “isocyanato” group refers to a “—NCO” group. A “thiocyanato” group refers to a “—SCN” group. An “isothiocyanato” group refers to an “—NCS” group.

A “sulfinyl” group refers to an “—S(O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C2-6 alkynyl, C₃₋₇ carbocyclyl, C₆₋₁₀ aryl, 5-10 membered heteroaryl, and 5-10 membered heterocyclyl, as defined herein.

An “N-sulfonamido” group refers to a “—N(R_{A})SO₂R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-carbamyl” group refers to a “—OC(O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})OC(O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-thiocarbamyl” group refers to a “—OC(S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-thiocarbamyl” group refers to an “—N(R_{A})OC(S)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A non-limiting example includes free amino (i.e., —NH₂).

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₂-C₆ alkenyl, C₂-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl (C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl (C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy (C₁-C₆)alkyl (i.e., ether), aryloxy, sulfhydryl (mercapto), halo (C₁-C₆)alkyl (e.g., —CF₃), halo (C₁-C₆)alkoxy (e.g., —OCF₃), C₁-C₆ alkylthio, arylthio, amino, amino (C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

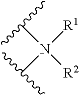

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

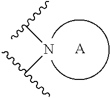

where ring A is a heteroaryl ring containing the depicted nitrogen.

Similarly, when two “adjacent” R groups are said to form a ring “together with the atom to which they are attached,” it is meant that the collective unit of the atoms, intervening bonds, and the two R groups are the recited ring. For example, when the following substructure is present:

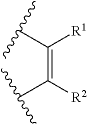

and R¹ and R² are defined as selected from the group consisting of hydrogen and alkyl, or R¹ and R² together with the atoms to which they are attached form an aryl or carbocylyl, it is meant that R¹ and can be selected from hydrogen or alkyl, or alternatively, the substructure has structure:

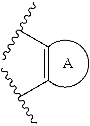

where A is an aryl ring or a carbocylyl containing the depicted double bond.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

The terms “modulate”, “modulator” and “modulation” of a proton-activated GPCR, as used herein, is intended to encompass antagonism, agonism, partial antagonism and/or partial agonism of an activity associated with a proton-activated GPCR. In various embodiments, “modulation” may inhibit or stimulate proton-activated GPCR activity. In certain embodiments, “modulation” refers to inhibition of proton-activated GPCR activity.

A “patient” or “subject” to be treated by the methods described herein may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease, disorder or condition. A subject regardless of whether a human or non-human animal may be referred to as an individual, subject, animal, host or recipient as well as patient.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice guinea pigs, or the like.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.)(1990); Goodman and Oilman's: The Pharmacological Basis of Therapeutics, 8th Ed., Pergamon Press.

A therapeutic effect relieves, to some extent, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease, disorder or condition, but who is susceptible to, or otherwise at risk of, a particular disease, disorder or condition, whereby the treatment reduces the likelihood that the patient will develop the disease, disorder or condition. The term “therapeutic treatment” refers to administering treatment to a subject already suffering from a disease, disorder or condition.

The term “at risk” as used herein, refers to a probability of developing a disease, disorder or condition described herein, that is higher than in the general population. Accordingly, treatment of an individual considered to be “at risk” of a particular condition is designed to prevent a subject from the developing the disease, disorder or condition or at least to reduce the risk of developing the disease, disorder or condition to a level no higher than that found in general population as a whole.

As used herein, “an effective amount” is intended to mean an amount sufficient to prevent, reduce, or eliminate a disease, disorder or condition. In some embodiments an effective amount can range from nanomolar concentrations to micromolar concentrations, for example, from about 1 nM to about 1,000 μM. One skilled in the art will recognize that to achieve these concentrations in vivo there will be a particular dependence on the pharmacokinetics of the exact compound selected. Thus, in some embodiments, a dose may range from about 1 mg/kg to about 1,000 mg/kg, including all quantities in between, and from about 10 mg/kg to about 500 mg/kg in other embodiments, and from about 50 mg/kg to about 250 mg/kg in yet other embodiments.

The terms “co-administration” or the like, as used herein, are meant to encompass administration of a compound described herein and an additional therapeutic agents to a single patient, and are intended to include treatment regimens in which the compounds and agents are administered by the same or different route of administration or at the same or different time.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### Compounds of the Disclosure

This disclosure is directed, in part, to compounds that are modulators, such as inhibitors, of proton-activated GPCRs, for example GPR68 (OGR1), which allows the compounds to be useful in prophylaxis, amelioration, treatment and/or prevention of diseases, disorders, or conditions associated with proton-activated GPCRs, such as inflammatory, fibrotic, or proliferative diseases, disorders, or conditions, and/or cancer.

Accordingly, in one aspect, there is provided a compound of formula (I):

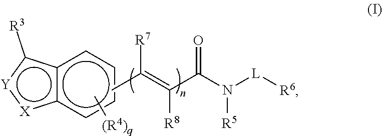

or a pharmaceutically acceptable salt, solvate or stereoisomer thereof,
wherein:
X is selected from N and NR¹;
Y is selected from N and NR²;
R¹ and R² are independently selected from H, C₁₋₁₀ alkyl, C₁₋₁₀ heteroalkyl, C₂₋₁₀ alkenyl, C₂₋₁₀ alkynyl, C₃₋₆ carbocyclyl, heterocyclyl, aryl, and heteroaryl, each of which is optionally substituted with one or more substituents selected from C₁₋₁₀ alkyl, halogen, —CN, OR⁹, —SR⁹, —S(O)₂R⁹, —NR⁹R¹⁰, —NO₂, ═(O), ═(S), ═(N)R⁹, ═(N)R¹⁰, —C(O)R⁹, —C(O)NR⁹R¹⁰, —C(O)SR⁹ or —C(O)OR⁹; or with one or more groups selected from C₃₋₆ carbocyclyl, heterocyclyl, aryl, and heteroaryl, each of which is optionally substituted with one or more substituents selected from C₁₋₁₀ alkyl, halogen, —CN, —OR⁹, —SR⁹, —S(O)₂R⁹, —NR⁹R¹⁰, —NO₂, ═(O), ═(S), —(N)R⁹, —(N)R¹⁰, —C(O)R⁹, —C(O) NR⁹R¹⁰, —C(O)SR⁹ or —C(O)OR⁹;
or R¹ and R², together with the atoms to which they are attached, form a fused 5-6 membered heteroaryl or a fused 5-6 membered heterocyclyl, each of which is optionally substituted with one or more substituents selected from optionally substituted C₁₋₁₀ alkyl, halogen, —CN, —OR⁹, —SR⁹, —S(O)₂R⁹, —NR⁹R¹⁰, —NO₂, ═(O), ═(S), ═(N)R⁹, ═(N)R¹⁰, —C(O)R⁹, —C(O)NR⁹R¹⁰, —C(O)SR⁹ or —C(O)OR⁹;
each R⁹ is independently selected from H, optionally substituted C₁₋₆ alkyl, and optionally substituted C₅₋₆ aryl;
each R¹⁰ is independently selected from H, optionally substituted C₁₋₆ alkyl, optionally substituted C₅₋₆ aryl, and —C(O)R⁹;
R³ is selected from H, halogen, hydroxy, optionally substituted C₁₋₆ alkyl, optionally substituted C₂₋₆ alkenyl, optionally substituted C₂₋₆ alkynyl, optionally substituted C₁₋₆ alkylthio, optionally substituted C₂₋₈ alkoxyalkyl, optionally substituted C₃₋₁₀ carbocyclyl, optionally substituted C₅₋₁₀ aryl, —CN, —OR⁹, —SR⁹, —NR⁹R¹⁰, —C(O)R⁹, —C(O)OR⁹, —S(O)₂R⁹, and —NO₂;
or R³ and R², together with the atoms to which they are attached, form a fused 5-6 membered heteroaryl or a fused 5-6 membered heterocyclyl, each of which is optionally substituted with one or more substituents selected from C₁₋₁₀ alkyl, halogen, —CN, —OR⁹, —SR⁹, —S(O)₂R⁹, —NR⁹R¹⁰, —NO₂, ═(O), ═(S), —(N)R⁹, ═(N)R¹⁰, —C(O)R⁹, C(O) NR⁹R¹⁰, —C(O)SR⁹ or —C(O)OR⁹;
q is an integer from 0 to 3;
R⁴ is selected from halogen, hydroxy, optionally substituted C₁₋₆ alkyl, optionally substituted C₂₋₆ alkenyl, optionally substituted C₂₋₆ alkynyl, optionally substituted C₁₋₆ alkylthio, optionally substituted C₂₋₈ alkoxyalkyl, optionally substituted C₃₋₁₀ carbocyclyl, optionally substituted C₆₋₁₀ aryl, —CN, —OR⁹, —SR⁹, —NR⁹R¹⁰, —C(O)R⁹, —C(O)OR⁹, —S(O)₂R⁹, and —NO₂;
R⁵ is selected from H, optionally substituted C₁₋₆ alkyl, optionally substituted C₂₋₆ alkenyl, optionally substituted C₂₋₆ alkynyl, optionally substituted C₂₋₈ alkoxyalkyl, optionally substituted C₃₋₆ carbocyclyl, optionally substituted C₅₋₁₀ aryl, optionally substituted 5-6 membered heteroaryl, and optionally substituted 5-6 membered heterocyclyl;
L is a bond, or a linker selected from —C(O)N(R⁹)—, —S(O)₂—, —C(O)—, or —C(O)O—; or from optionally substituted C₁₋₄ alkylene, optionally substituted C₂₋₄ alkenylene, —OC₁₋₄ alkylene, or —OC₂₋₄ alkenylene, each of which is optionally interrupted with —O—, —S—, —NR⁹—, —NR¹⁰—, —C(O)N(R⁹)—, —N(R⁹) C(O)—, —S(O)₂—, —N(R⁹) S(O)₂—, —S(O)₂N(R⁹)—, —C(O)—, —C(O)O—, or —O(O)C—;
R⁶ is selected from C₅₋₁₀ aryl, 5-10 membered heteroaryl, 3-10 membered heterocyclyl, C₃₋₁₀ carbocyclyl, C₁₋₁₀ alkyl, C₂₋₁₀ alkenyl, or C₂₋₁₀ alkynyl, each of which is optionally substituted with one or more substituents, which may be selected from optionally substituted C₁₋₆ alkyl, optionally substituted C₅₋₆ aryl, optionally substituted 5-6 membered heteroaryl, optionally substituted 5-6 membered heterocyclyl, halogen, —CN, —OR¹¹, —SR¹¹, —S(O)₂R¹¹, —S(O)₂N(R¹¹)₂, —S(O)(NR¹⁰)R¹¹, —C(O)N(R¹¹)₂, —NR¹¹C(O)R¹¹, —N(R¹¹) S(O)₂R¹¹, —N(R¹¹)₂, —NO₂, ═(O), ═(S), ═(N)R¹¹, C(O)R¹¹, —C(O)OR¹¹, —C(O)N(R¹¹)₂, or —C(O)SR¹¹; wherein two adjacent optional substituents, when present, together with the atoms to which they are attached, may form an optionally substituted fused 5-6 membered heteroaryl or an optionally substituted fused 5-6 membered heterocyclyl;
or R⁶ and R⁵, together with the atoms to which they are attached, form a 3-10 membered heterocyclyl or 5-10 membered heteroaryl, each of which is optionally substituted with one or more substituents which may be selected from optionally substituted C₁₋₁₀ alkyl, optionally substituted C₂₋₆ alkenyl, optionally substituted C₂₋₆ alkynyl, halogen, —CN, —OR¹¹, —SR¹¹, —S(O)₂R¹¹, —S(O)₂N(R¹¹)₂, —S(O)(NR¹⁰)R¹¹, —C(O) N(R¹¹)₂, —NR¹¹C(O)R¹¹, —N(R¹¹) S(O)₂R¹¹, —N(R¹¹)₂, —NO₂,═(O), ═(S), ═(N)R¹¹, —C(O)R¹¹, —C(O)OR¹¹, —C(O)N(R¹¹)₂, or —C(O)SR¹¹;
each R¹¹ is independently selected from H, optionally substituted C₁₋₆ alkyl, optionally substituted C₂₋₆ alkenyl, optionally substituted C₂₋₆ alkynyl, optionally substituted C₅₋₁₀ aryl, optionally substituted C₂₋₂₀ alkoxyalkyl, C₁-C₆ haloalkyl, —C(O)R⁹, —OR⁹, —S(O)₂R⁹, and —S(O)₂N(R⁹)₂.
R⁷ and R⁸ are independently selected from H, C₁₋₆ alkyl, C₅₋₁₀ aryl, 5-10 membered heteroaryl, and C₃₋₁₀ carbocyclyl, each of which is optionally substituted with one or more substituents selected from C₁₋₆ alkyl, C₅₋₆ aryl, halogen, —CN, —OR⁹, —SR⁹, —S(O)₂R⁹, —NR⁹R¹⁰, —NO₂, ═(O), ═(S), ═(N)R⁹, ═(N)R¹⁰, —C(O)R⁹, —C(O)NR⁹R¹⁰, —C(O)SR⁹ or —C(O)OR⁹; and
n is an integer from 1 to 4.

In some embodiments, n is 4. In some embodiments, n is 3. In some embodiments, n is 2. In some embodiments, n is 1, and the compound is having the structure of Formula (Ia):

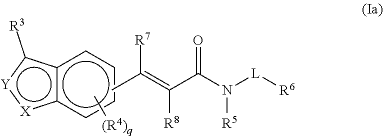

or a pharmaceutically acceptable salt, solvate or stereoisomer thereof.

In some embodiments, the compound is having the structure of Formula (II):

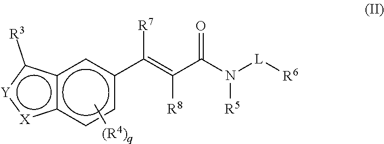

or a pharmaceutically acceptable salt, solvate or stereoisomer thereof.

In some embodiments, the compound is having the structure of Formula (IIa):

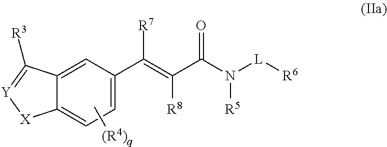

or a pharmaceutically acceptable salt, solvate or stereoisomer thereof,
wherein X is NR¹; and Y is N.

In other embodiments, the compound is having the structure of Formula (IIb):

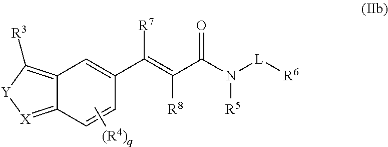

or a pharmaceutically acceptable salt, solvate or stereoisomer thereof,
wherein X is N; and Y is NR².

In some embodiments, R¹ and R² are independently selected from H, C₁₋₁₀ alkyl, C₂₋₁₀ alkenyl, C₂₋₁₀ alkynyl, or C₃₋₆ carbocyclyl, each of which is optionally substituted with one or more substituents selected from C₁₋₆ alkyl, halogen, —CN, OR⁹, and —SR⁹; or with one or more groups selected from C₃₋₆ carbocyclyl, 5-6 membered heterocyclyl, C₅₋₆ aryl, and 5-6 membered heteroaryl, each of which is optionally substituted with one or more substituents selected from C₁₋₆ alkyl, halogen, —CN, —OR⁹, —SR⁹, —S(O)₂R⁹, —NR⁹R¹⁰, —NO₂, ═(O), —(S), —(N)R⁹, —(N)R¹⁰, —C(O)R⁹, —C(O) NR⁹R¹⁰, —C(O)SR⁹ or —C(O)OR⁹.

In some embodiments, R¹ and/or R² is C₁₋₆ alkyl which is optionally substituted with one or more substituents selected from C₁₋₆ alkyl, halogen, —CN, —OR⁹, and —SR⁹; or with one or more groups selected from C₃₋₆ carbocyclyl, 5-6 membered heterocyclyl, C₅₋₆ aryl, and 5-6 membered heteroaryl, each of which is optionally substituted with one or more substituents selected from C₁₋₆ alkyl, halogen, —CN, —OR⁹, —SR⁹, —S(O)₂R⁹, —NR⁹R¹⁰, —NO₂, —(O), —(S), —(N)R⁹, ═(N)R¹⁰, —C(O)R⁹, —C(O) NR⁹R¹⁰, —C(O)SR⁹ or —C(O)OR⁹.

In some embodiments, R¹ and/or R² is C₁₋₆ alkyl which is optionally selected from methyl, ethyl, propyl or isopropyl.

In some embodiments, R¹ and/or R² is C₁₋₆ alkyl which is substituted with halogen, —CN, —OR⁹, —C(O)R⁹, —C(O)NR⁹R¹⁰, C(O)SR⁹ or —C(O)OR⁹, optionally wherein R⁹ is methyl.

In some embodiments, R¹ and/or R² is C₁₋₆ alkyl which is substituted with optionally substituted 5-6 membered heteroaryl or optionally substituted 3-6 membered heterocyclyl.

In some embodiments, R¹ and/or R² is C₁₋₄ alkyl which is substituted with a 3-6 membered heterocyclyl selected from:

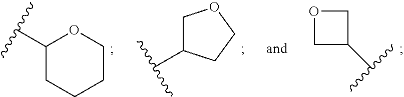 [SMILES: *C1CCCCO1 ; *C1CCOC1 ; *C1COC1]

each of which may be optionally substituted.

As will be clear to a skilled artisan, the optionally substituted heterocyclyl group may have defined stereochemistry at one or more chiral centers, when present. For example, in some embodiments, R¹ and/or R² is C₁₋₄ alkyl which is substituted with a 3-6 membered heterocyclyl selected from:

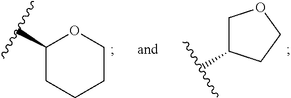 [SMILES: *[C@@H]1CCCCO1 ; *[C@H]1CCOC1]

each of which may be optionally substituted.

In some embodiments, R¹ and/or R² is C₁₋₆ alkyl which is substituted with optionally substituted C₁₋₆ carbocyclyl. In some embodiments, R¹ and R² are independently selected from H and C1-4 alkyl which is substituted with C1-6 carbocyclyl, optionally wherein C₁₋₆ carbocyclyl is selected from optionally substituted cyclobutyl or cyclopropyl. In some embodiments, R¹ and/or R² is

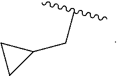 [SMILES: *CC1CC1]

In various embodiments, there is provided a compound of Formula (III):

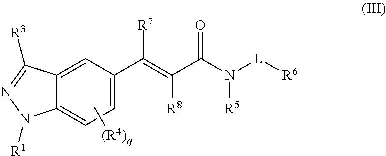

or a pharmaceutically acceptable salt, solvate or stereoisomer thereof,
wherein:
R¹ is selected from H, C₁₋₁₀ alkyl, C₁₋₁₀ heteroalkyl, C₂₋₁₀ alkenyl, C₂₋₁₀ alkynyl, C₃₋₆ carbocyclyl, heterocyclyl, aryl, and heteroaryl, each of which is optionally substituted with one or more substituents selected from C₁₋₁₀ alkyl, halogen, —CN, —OR⁹, —SR⁹, —S(O)₂R⁹, —NR⁹R¹⁰, —NO₂, ═(O), —(S), —(N)R⁹, —(N)R¹⁰, —C(O)R⁹, —C(O) NR⁹R¹⁰, —C(O)SR⁹ or —C(O)OR⁹; or with one or more groups selected from C₃₋₆ carbocyclyl, heterocyclyl, aryl, and heteroaryl, each of which is optionally substituted with one or more substituents selected from C₁₋₁₀ alkyl, halogen, —CN, —OR⁹, —SR⁹, —S(O)₂R⁹, —NR⁹R¹⁰, —NO₂, —(O), ═(S), ═(N)R⁹, —(N)R¹⁰, —C(O)R⁹, —C(O)NR⁹R¹⁰, —C(O)SR⁹ or —C(O)OR⁹;
or R¹ and R², together with the atoms to which they are attached, form a fused 5-6 membered heteroaryl or a fused 5-6 membered heterocyclyl, each of which is optionally substituted with one or more substituents selected from optionally substituted C₁₋₁₀ alkyl, halogen, —CN, —OR⁹, —SR⁹, —S(O)₂R⁹, —NR⁹R¹⁰, —NO₂, ═(O), ═(S), —(N)R⁹, ═(N)R¹⁰, —C(O)R⁹, —C(O)NR⁹R¹⁰, —C(O)SR⁹ or —C(O)OR⁹;
each R⁹ is independently selected from H, optionally substituted C₁₋₆ alkyl, and optionally substituted C₅₋₆ aryl;
each R¹⁰ is independently selected from H, optionally substituted C₁₋₆ alkyl, optionally substituted C₅₋₆ aryl, and —C(O)R⁹;
R³ is selected from H, halogen, hydroxy, optionally substituted C₁₋₆ alkyl, optionally substituted C₂₋₆ alkenyl, optionally substituted C₂₋₆ alkynyl, optionally substituted C₁₋₆ alkylthio, optionally substituted C₂₋₈ alkoxyalkyl, optionally substituted C₃₋₁₀ carbocyclyl, optionally substituted C₅₋₁₀ aryl, —CN, —OR⁹, —SR⁹, —NR⁹R¹⁰, —C(O)R⁹, —C(O)OR⁹, —S(O)₂R⁹, and —NO₂;
or R³ and R², together with the atoms to which they are attached, form a fused 5-6 membered heteroaryl or a fused 5-6 membered heterocyclyl, each of which is optionally substituted with one or more substituents selected from C₁₋₁₀ alkyl, halogen, —CN, —OR⁹, —SR⁹, —S(O)₂R⁹, —NR⁹R¹⁰, —NO₂, ═(O), ═(S), ═(N)R⁹, ═(N)R¹⁰, —C(O)R⁹, C(O) NR⁹R¹⁰, —C(O)SR⁹ or —C(O)OR⁹;
q is an integer from 0 to 3;
R⁴ is selected from halogen, hydroxy, optionally substituted C₁₋₆ alkyl, optionally substituted C₂₋₆ alkenyl, optionally substituted C₂₋₆ alkynyl, optionally substituted C₁₋₆ alkylthio, optionally substituted C₂₋₈ alkoxyalkyl, optionally substituted C₃₋₁₀ carbocyclyl, optionally substituted C₆₋₁₀ aryl, —CN, —OR⁹, —SR⁹, —NR⁹R¹⁰, —C(O)R⁹, —C(O)OR⁹, —S(O)₂R⁹, and —NO₂;
R⁵ is selected from H, optionally substituted C₁₋₆ alkyl, optionally substituted C₂₋₆ alkenyl, optionally substituted C₂₋₆ alkynyl, optionally substituted C₂₋₈ alkoxyalkyl, optionally substituted C₃₋₆ carbocyclyl, optionally substituted C₅₋₁₀ aryl, optionally substituted 5-6 membered heteroaryl, and optionally substituted 5-6 membered heterocyclyl;
L is a bond, or a selected from —C(O)N(R⁹)—, —S(O)₂—, —C(O)—, or —C(O)O—; or from optionally substituted C₁₋₄ alkylene, optionally substituted C₂₋₄ alkenylene, —OC₁₋₄ alkylene, or —OC₂₋₄ alkenylene, each of which is optionally interrupted with —O—, —S—, —NR⁹-, —NR¹⁰—, —C(O)N(R⁹)—, —N(R⁹) C(O)—, —S(O)₂—, —N(R⁹) S(O)₂—, —S(O)₂N(R⁹)—, —C(O)—, —C(O)O—, or —O(O)C—;
R⁶ is selected from C₅₋₁₀ aryl, 5-10 membered heteroaryl, 3-10 membered heterocyclyl, C₃₋₁₀ carbocyclyl, C₁₋₁₀ alkyl, C₂₋₁₀ alkenyl, or C₂₋₁₀ alkynyl, each of which is optionally substituted with one or more substituents which may be selected from optionally substituted C₁₋₆ alkyl, optionally substituted C₅₋₆ aryl, optionally substituted 5-6 membered heteroaryl, optionally substituted 5-6 membered heterocyclyl, halogen, —CN, —OR¹¹, —SR¹¹, —S(O)₂R¹¹, —S(O)₂N(R¹¹)₂, —S(O)(NR¹⁰)R¹¹, —C(O)N(R¹¹)₂, —NR¹¹C(O)R¹¹, —N(R¹¹) S(O)₂R¹¹, —N(R¹¹)₂, —NO₂,═(O),═(S), ═(N)R¹¹, —C(O)R¹¹, —C(O)OR¹¹, —C(O)N(R¹¹)₂, or —C(O)SR¹¹; wherein two adjacent optional substituents, when present, together with the atoms to which they are attached, may form an optionally substituted fused 5-6 membered heteroaryl or an optionally substituted fused 5-6 membered heterocyclyl;
or R⁶ and R⁵, together with the atoms to which they are attached, form a 3-10 membered heterocyclyl or 5-10 membered heteroaryl, each of which is optionally substituted with one or more substituents which may be selected from optionally substituted C₁₋₁₀ alkyl, optionally substituted C₂₋₆ alkenyl, optionally substituted C₂₋₆ alkynyl, halogen, —CN, —OR¹¹, —SR¹¹, —S(O)₂R¹¹, —S(O)₂N(R¹¹)₂, —S(O)(NR¹⁰)R¹¹, —C(O) N(R¹¹)₂, —NR¹¹C(O)R¹¹, —N(R¹¹) S(O)₂R¹¹, —N(R¹¹)₂, —NO₂, ═(O), ═(S), ═(N)R¹¹, —C(O)R¹¹, —C(O)OR¹¹, —C(O)N(R¹¹)₂, or —C(O)SR¹¹;
each R¹¹ is independently selected from H, optionally substituted C₁₋₆ alkyl, optionally substituted C₂₋₆ alkenyl, optionally substituted C₂₋₆ alkynyl, optionally substituted C₅₋₁₀ aryl, optionally substituted C₂₋₂₀ alkoxyalkyl, C₁-C₆ haloalkyl, —C(O)R⁹, —OR⁹, —S(O)₂R⁹, and —S(O)₂N(R⁹) 2.
R⁷ and R⁸ are independently selected from H, C₁₋₆ alkyl, C₅₋₁₀ aryl, 5-10 membered heteroaryl, and C₃₋₁₀ carbocyclyl, each of which is optionally substituted with one or more substituents selected from C₁₋₆ alkyl, C₅₋₆ aryl, halogen, —CN, —OR⁹, —SR⁹, —S(O)₂R⁹, —NR⁹R¹⁰, —NO₂, ═(O), ═(S), ═(N)R⁹, ═(N)R¹⁰, —C(O)R⁹, —C(O)NR⁹R¹⁰, C(O)SR⁹ or —C(O)OR⁹.

In some embodiments, R⁷ and R⁸ are independently selected from C₁₋₆ alkyl, C₅₋₁₀ aryl, 5-10 membered heteroaryl, and C₃₋₁₀ carbocyclyl, each of which is optionally substituted with one or more substituents selected from C₁₋₆ alkyl, C₅₋₆ aryl, halogen, —CN, —OR⁹, —SR⁹, —S(O)₂R⁹, —NR⁹R¹⁰, —NO₂, ═(O), ═(S), ═(N)R⁹, ═(N)R¹⁰, —C(O)R⁹, —C(O) NR⁹R¹⁰, —C(O)SR⁹ or —C(O)OR⁹.

In some embodiments, R⁷ and R⁸ are H, and the compound is having the structure of Formula (IV):

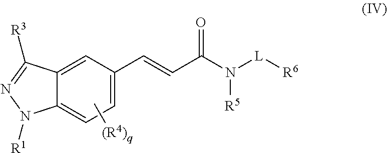

or a pharmaceutically acceptable salt, solvate or stereoisomer thereof.

In some embodiments, R¹ is C₁₋₁₀ alkyl, C₂₋₁₀ alkenyl, C₂₋₁₀ alkynyl, or C₃₋₆ carbocyclyl, each of which is optionally substituted with one or more substituents selected from C₁₋₆ alkyl, halogen, —CN, OR⁹, and —SR⁹; or with one or more groups selected from C₃₋₆ carbocyclyl, 5-6 membered heterocyclyl, C₅₋₆ aryl, and 5-6 membered heteroaryl, each of which is optionally substituted with one or more substituents selected from C₁₋₆ alkyl, halogen, —CN, OR⁹, —SR⁹, —S(O)₂R⁹, —NR⁹R¹⁰, —NO₂,═(O),═(S), ═(N)R⁹, —(N)R¹⁰, —C(O)R⁹, —C(O)NR⁹R¹⁰, —C(O)SR⁹ or —C(O)OR⁹.

In some embodiments, R¹ is C₁₋₆ alkyl, optionally substituted with one or more substituents selected from C₁₋₆ alkyl, halogen, —CN, —OR⁹, and —SR⁹; or with one or more groups selected from C₃₋₆ carbocyclyl, 5-6 membered heterocyclyl, C₅₋₆ aryl, and 5-6 membered heteroaryl, each of which is optionally substituted with one or more substituents selected from C₁₋₆ alkyl, halogen, —CN, —OR⁹, —SR⁹, —S(O)₂R⁹, —NR⁹R¹⁰, —NO₂,═(O),═(S), ═(N)R⁹, ═(N)R¹⁰, —C(O)R⁹, —C(O)NR⁹R¹⁰, —C(O)SR⁹ or —C(O)OR⁹.

In some embodiments, R¹ is C₁₋₆ alkyl. In some embodiments, R¹ is methyl, ethyl, propyl or isopropyl.

In some embodiments, R¹ is C₁₋₆ alkyl substituted with halogen, —CN, —OR⁹, —C(O)R⁹, —C(O)NR⁹R¹⁰, C(O)SR⁹ or —C(O)OR⁹, wherein in some embodiments R⁹ is methyl.

In some embodiments, R¹ is C₁₋₆ alkyl substituted with optionally substituted 5-6 membered heteroaryl or optionally substituted 3-6 membered heterocyclyl. For example, R¹ may be C1-4 alkyl substituted with a 3-6 membered heterocyclyl selected from:

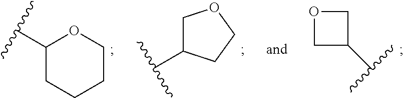 [SMILES: *C1CCCCO1 ; *C1CCOC1 ; *C1COC1]

each of which may be optionally substituted.

As will be clear to a skilled artisan, the optionally substituted heterocyclyl group may have defined stereochemistry at one or more chiral centers, when present. For example, in some embodiments, R¹ is C₁₋₄ alkyl substituted with a 3-6 membered heterocyclyl selected from:

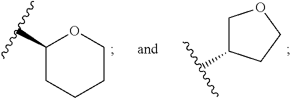 [SMILES: *[C@@H]1CCCCO1 ; *[C@H]1CCOC1]

each of which may be optionally substituted.

In some embodiments, R¹ is C₁₋₆ alkyl substituted with optionally substituted C₁₋₆ carbocyclyl. In some embodiments, R¹ is C₁₋₄ alkyl substituted with C1-6 carbocyclyl, optionally wherein C₁₋₆ carbocyclyl is selected from optionally substituted cyclobutyl or optionally substituted cyclopropyl.

In some embodiments, R¹ is

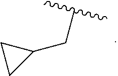 [SMILES: *CC1CC1]

In other embodiments, R¹ and R², together with the atoms to which they are attached, form a fused 5-6 membered heteroaryl or a fused 5-6 membered heterocyclyl, each of which is optionally substituted with one or more substituents selected from optionally substituted C₁₋₁₀ alkyl, halogen, —CN, —OR⁹, —SR⁹, —S(O)₂R⁹, —NR⁹R¹⁰, —NO₂, —(O),═(S), ═(N)R⁹, —(N)R¹⁰, —C(O)R⁹, —C(O)NR⁹R¹⁰, —C(O)SR⁹ or —C(O)OR⁹.

In some embodiments, R³ is halogen, hydroxy, or optionally substituted C₁₋₆ alkyl, such as for example —CF₃ or —CH₂CF₃. In some embodiments, R³ is C₁₋₄ alkyl. In some embodiments, R³ is optionally substituted C₁₋₆ alkylthio, optionally substituted C₂₋₈ alkoxyalkyl, optionally substituted C₃₋₁₀ carbocyclyl, or optionally substituted C₅₋₁₀ aryl. In some embodiments, R³ is-CN, —C(O)R⁹, —C(O)OR⁹, —S(O)₂R⁹, or —NO₂. In some embodiments, R³ is-SR⁹, —NR⁹R¹⁰, or —OR⁹, such as for example —OMe or —OCF₃.

In some embodiments, R³ is H.

In some embodiments, R³ and R², together with the atoms to which they are attached, form a fused 5-6 membered heteroaryl or a fused 5-6 membered heterocyclyl, each of which is optionally substituted with one or more substituents selected from C₁₋₁₀ alkyl, halogen, —CN, —OR⁹, —SR⁹, —S(O)₂R⁹, —NR⁹R¹⁰, —NO₂, ═(O), ═(S), ═(N)R⁹, ═(N)R¹⁰, —C(O)R⁹, —C(O)NR⁹R¹⁰, —C(O)SR⁹ or —C(O)OR⁹.

In some embodiments, q is 3. In some embodiments, q is 2. In some embodiments, q is 1. In some embodiments, q is 0.

In some embodiments, q is 0 or 1. In some embodiments, the compound is having the structure of Formula (Va), (Vb) or (Vc):

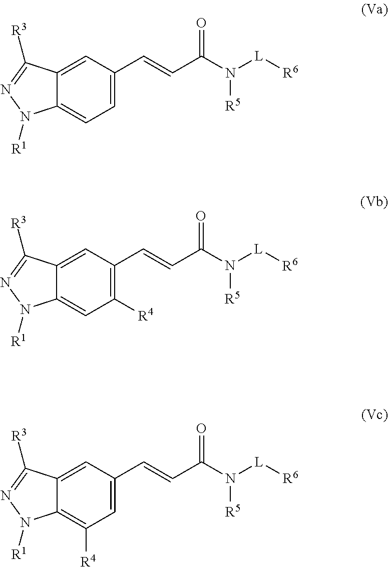

or a pharmaceutically acceptable salt, solvate or stereoisomer thereof.

In some embodiments, R⁴ is selected from halogen, hydroxy, optionally substituted C₂₋₈ alkoxyalkyl, —CN, —OR⁹, —SR⁹, —NR⁹R¹⁰, —C(O)R⁹, —C(O)OR⁹, —S(O)₂R⁹, and —NO₂.

In some embodiments, R⁴ is selected from halogen, —CN, and —NO₂. In some embodiments, R⁴ is halogen. In some embodiments, R⁴ is F.

In some embodiments, L is a linker selected from —C(O)N(R⁹)—, —S(O)₂—, —C(O)—, or —C(O)O—; or from optionally substituted C₁₋₄ alkylene, optionally substituted C₂₋₄ alkenylene, —OC₁₋₄ alkylene, or —OC₂₋₄ alkenylene, each of which is optionally interrupted with —O—, —S—, —NR⁹—, -NR¹⁰_, —C(O)N(R⁹)—, —N(R⁹) C(O)—, —S(O)₂, —N(R⁹) S(O)₂—, —S(O)₂N(R⁹)—, —C(O)—, C(O)O—, or —O(O)C—. In certain embodiments, the linker is selected from —S(O) 2—, C(O)—, —C(O)O—. In certain embodiments, the linker is optionally substituted C₁₋₄ alkylene. In certain embodiments, the linker is C1-4 alkylene interrupted by —S(O)₂—, —C(O)—, —C(O)O—, or —O(O)C—.

In some embodiments, L is a bond, and the compound is having the structure of Formula (VI):

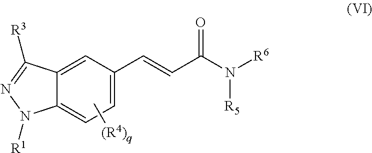

or a pharmaceutically acceptable salt, solvate or stereoisomer thereof.

In some embodiments, q is 0 or 1. In some embodiments, the compound is having the structure of Formula (VIa), (VIb) or (VIc):

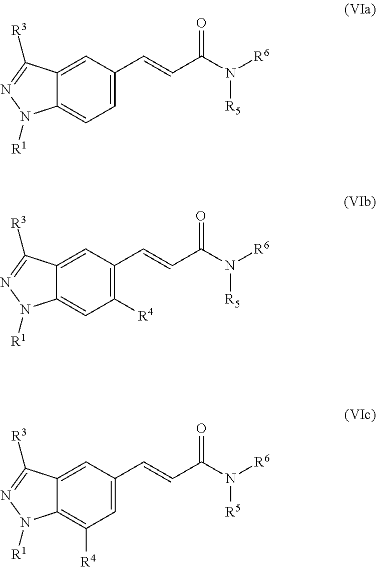

or a pharmaceutically acceptable salt, solvate or stereoisomer thereof.

In some embodiments, R⁴ is selected from halogen, hydroxy, optionally substituted C₂₋₈ alkoxyalkyl, —CN, —OR⁹, —SR⁹, —NR⁹R¹⁰, —C(O)R⁹, —C(O)OR⁹, —S(O)₂R⁹, and —NO₂.

In some embodiments, R⁴ is selected from halogen, —CN, and —NO₂. In some embodiments, halogen is F.

In some embodiments, R⁵ is H, optionally substituted C₁₋₄ alkyl, optionally substituted C₂₋₄ alkenyl, optionally substituted C₂₋₄ alkynyl, or optionally substituted C₂₋₈ alkoxyalkyl.

In some embodiments, R⁵ is H.

In some embodiments, R⁶ is selected from C₅₋₁₀ aryl, 5-10 membered heteroaryl, 3-10 membered heterocyclyl, C₃₋₁₀ carbocyclyl, C₁₋₁₀ alkyl, C₂₋₁₀ alkenyl, or C₂₋₁₀ alkynyl, each of which is optionally substituted with one or more substituents which may be selected from optionally substituted C₁₋₆ alkyl, optionally substituted C₅₋₆ aryl, optionally substituted 5-6 membered heteroaryl, optionally substituted 5-6 membered heterocyclyl, halogen, —CN, —OR¹¹, —SR¹¹, —S(O)₂R¹¹, —S(O)₂N(R¹¹)₂, —S(O)(NR¹⁰)R¹¹, —C(O)N(R¹¹)₂, —NR¹¹C(O)R¹¹, —N(R¹¹) S(O)₂R¹¹, —N(R¹¹)₂, —NO₂, ═(O), ═(S), ═(N)R¹¹, —C(O)R¹¹, —C(O)OR¹¹, —C(O)N(R¹¹)₂, or —C(O)SR¹¹; wherein two adjacent optional substituents, when present, together with the atoms to which they are attached, may form an optionally substituted fused 5-6 membered heteroaryl or an optionally substituted fused 5-6 membered heterocyclyl.

In some embodiments, R⁶ is selected from optionally substituted C₅₋₁₀ aryl and optionally substituted 5-10 membered heteroaryl.

In some embodiments, R⁶ is optionally substituted C₅₋₁₀ aryl, such as for example phenyl, naphthyl, or azulenyl, each of which may be optionally substituted. In some embodiments, R⁶ is an optionally substituted phenyl.

In some embodiments, R⁶ is optionally substituted 5-10 membered heteroaryl. In some embodiments, the 5-10 membered heteroaryl is monocyclic heteroaryl, such as for example furanyl, thienyl, pyrrolyl, oxazolyl, thiazolyl, imidazolyl, oxadiazolyl, thiadiazolyl, pyridyl, triazolyl, triazinyl, pyridazyl, isothiazolyl, isoxazolyl, pyrazinyl, pyrazolyl, or pyrimidinyl, each of which may be optionally substituted. In some embodiments, the 5-10 membered heteroaryl is bicyclic heteroaryl, such as for example quinoxalinyl, quinazolinul, pyridopyrazinyl, benzoxazolyl, benzothiophenyl, benzimidazolyl, naphthyridinyl, quinolinyl, benzofuranyl, indolyl, indazolyl, benzothiazolyl, oxazolyl [4,5-b]pyridyl, pyridopyrimidinyl, isoquinolinyl, and benzohydroxazole, each of which may be optionally substituted.

In certain embodiments, R⁶ is optionally substituted C6 aryl, optionally substituted 5 membered heteroaryl, or optionally substituted 6 membered heteroaryl.

In certain embodiments, R⁶ is selected from:

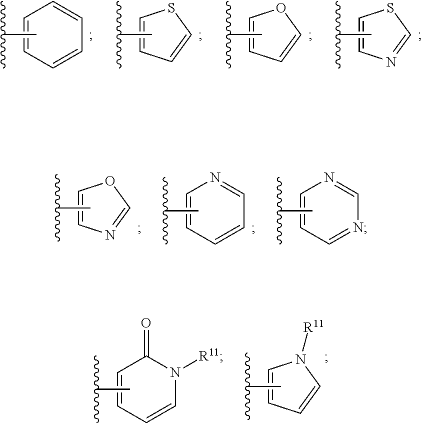

each of which may be optionally substituted.

In certain embodiments, R⁶ is selected from:

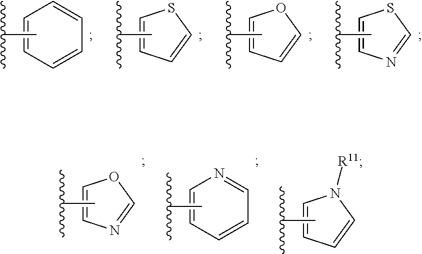

each of which may be optionally substituted.

In some embodiments, R⁶ groups described above, selected from C₅₋₁₀ aryl and 5-10 membered heteroaryl, are independently substituted with one or more substituents selected from the group comprising optionally substituted C₁₋₆ alkyl, optionally substituted C₅₋₆ aryl, optionally substituted 5-6 membered heteroaryl, optionally substituted 5-6 membered heterocyclyl, halogen, —CN, —OR¹¹, —SR¹¹, —S(O)₂R¹¹, —S(O)₂N(R¹¹)₂, —S(O)(NR¹⁰)R¹¹, —C(O)N(R¹¹)₂, —NR¹¹C(O)R¹¹, —N(R¹¹) S(O)₂R¹¹, —N(R¹¹)₂, —NO₂,═(O),═(S), —(N)R¹¹, —C(O)R¹¹, —C(O)OR¹¹, —C(O)N(R¹¹)₂, and —C(O)SR¹¹; wherein two adjacent optional substituents, when present, together with the atoms to which they are attached, may form an optionally substituted fused 5-6 membered heteroaryl or an optionally substituted fused 5-6 membered heterocyclyl. In some embodiments, the one or more substituents are selected from the group consisting of halogen, optionally substituted 5 membered heterocyclyl, optionally substituted 5 membered heteroaryl, —C(O)OR¹¹, —N(R¹¹) S(O)₂R¹¹, —C(O)N(R¹¹)₂, and —S(O)₂N(R¹¹)₂.

In some embodiments, R⁶ is selected from optionally substituted 3-10 membered heterocyclyl and optionally substituted C₃₋₁₀ carbocyclyl.

In some embodiments, R⁶ is selected from optionally substituted 3-10 membered heterocyclyl. In some embodiments, the 3-10 membered heterocyclyl is monocyclic heterocyclyl, such as for example aziridinyl, azetidinyl, pyrrolidinyl, imidazolidinyl, pyrazolidinyl, piperidinyl, piperazinyl, tetrahydrofuranyl, tetrahydropyranyl, morpholinyl, or piperidinonyl, each of which may be optionally substituted. In some embodiments, the 3-10 membered heterocyclyl is bicyclic heterocyclyl, such as dihydrobenzofuranyl, indanyl, indolinyl, isoindolinyl, tetrahydroisoquinolinyl, tetrahydroquinolyl, or benzoazepanyl, each of which may be optionally substituted.

In some embodiments, R⁶ is C₃₋₁₀ carbocyclyl, such as for example cyclopropyl, cyclobutyl, cyclopentyl, cyclohexyl, cyclohexenyl, 2,3-dihydro-indene, bicycle [2.2.2]octanyl, adamantyl, norbornyl, or spiro[4.4]nonanyl, each of which may be optionally substituted.

In some embodiments, R⁶ groups described above, selected from optionally substituted 3-10 membered heterocyclyl and optionally substituted C₃₋₁₀ carbocyclyl, are independently substituted with one or more substituents selected from the group comprising optionally substituted C₁₋₆ alkyl, optionally substituted C₅₋₆ aryl, optionally substituted 5-6 membered heteroaryl, optionally substituted 5-6 membered heterocyclyl, halogen, —CN, —OR¹¹, —SR¹¹, —S(O)₂R¹¹, —S(O)₂N(R¹¹)₂, —S(O)(NR¹⁰)R¹¹, C(O) N(R¹¹)₂, —NR¹¹C(O)R¹¹, —N(R¹¹) S(O)₂R¹¹, —N(R¹¹)₂, —NO₂,═(O), ═(S), ═(N)R¹¹, —C(O)R¹¹, —C(O)OR¹¹, —C(O)N(R¹¹)₂, and —C(O)SR¹¹; wherein two adjacent optional substituents, when present, together with the atoms to which they are attached, may form an optionally substituted fused 5-6 membered heteroaryl or an optionally substituted fused 5-6 membered heterocyclyl. In some embodiments, the one or more substituents are selected from the group consisting of halogen, —CN, —NO₂, and —C(O)OR¹¹, wherein R¹¹ may be H or optionally substituted C₁₋₆ alkyl. In some embodiments, the one or more substituent is halogen, —C(O)OH, or —C(O)OC₁₋₆ alkyl.

In some embodiments, R⁶ and R⁵, together with the atoms to which they are attached, form a 3-10 membered heterocyclyl or 5-10 membered heteroaryl, each of which is optionally substituted with one or more substituents which may be selected from optionally substituted C₁₋₁₀ alkyl, optionally substituted C₂₋₆ alkenyl, optionally substituted C₂₋₆ alkynyl, halogen, —CN, —OR¹¹, —SR¹¹, —S(O)₂R¹¹, —S(O)₂N(R¹¹)₂, —S(O)(NR¹⁰)R¹¹, —C(O)N(R¹¹)₂, —NR¹¹C(O)R¹¹, —N(R¹¹) S(O)₂R¹¹, —N(R¹¹)₂, —NO₂, ═(O), ═(S), ═(N)R¹¹, —C(O)R¹¹, —C(O)OR¹¹, —C(O)N(R¹¹)₂, or —C(O)SR¹¹.

In some embodiments, R⁶ and R⁵, together with the atoms to which they are attached, form an optionally substituted 3-10 membered heterocyclyl. The optionally substituted 3-10 membered heterocyclyl may be monocyclic heterocyclyl such as, for example, aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl or azepanyl, each of which may be optionally substituted. Alternatively, the optionally substituted 3-10 membered heterocyclyl may be bicyclic heterocyclyl such as, for example, dihydrobenzofuranyl, indanyl, indolinyl, isoindolinyl, tetrahydroisoquinolinyl, tetrahydroquinolyl, and benzoazepanyl, each of which may be optionally substituted.

In some embodiments, R⁶ and R⁵, together with the atoms to which they are attached, form an optionally substituted 5-10 membered heteroaryl. In some embodiments, the optionally substituted 5-10 membered heteroaryl is monocyclic heteroaryl, such as for example furanyl, thienyl, pyrrolyl, oxazolyl, thiazolyl, imidazolyl, oxadiazolyl, thiadiazolyl, pyridyl, triazolyl, triazinyl, pyridazyl, isothiazolyl, isoxazolyl, pyrazinyl, pyrazolyl, and pyrimidinyl, each of which may be optionally substituted. In some embodiments, the optionally substituted 5-10 membered heteroaryl is bicyclic heteroaryl, such as for example quinoxalinyl, quinazolinul, pyridopyrazinyl, benzoxazolyl, benzothiophenyl, benzimidazolyl, naphthyridinyl, quinolinyl, benzofuranyl, indolyl, indazolyl, benzothiazolyl, oxazolyl [4,5-b]pyridyl, pyridopyrimidinyl, isoquinolinyl, and benzohydroxazole, each of which may be optionally substituted.

In some embodiments, the groups formed by R⁶ and R⁵ together with the atoms to which they are attached, selected from optionally substituted 3-10 membered heterocyclyl and optionally substituted 5-10 membered heteroaryl, are independently substituted with one or more substituents selected from the group comprising optionally substituted C₁₋₆ alkyl, optionally substituted C₅₋₆ aryl, optionally substituted 5-6 membered heteroaryl, optionally substituted 5-6 membered heterocyclyl, halogen, —CN, —OR¹¹, —SR¹¹, —S(O)₂R¹¹, —S(O)₂N(R¹¹)₂, —S(O)(NR¹⁰)R¹¹, —C(O)N(R¹¹)₂, —NR¹¹C(O)R¹¹, —N(R¹¹) S(O)₂R¹¹, —N(R¹¹)₂, —NO₂,═(O), ═(S), ═(N)R¹¹, —C(O)R¹¹, —C(O)OR¹¹, —C(O)N(R¹¹)₂, and —C(O)SR¹¹. In some embodiments, the one or more substituents are selected from the group consisting of halogen, —CN, —NO₂, and —C(O)OR¹¹, wherein R¹¹ may be H or optionally substituted C₁₋₆ alkyl. In some embodiments, the one or more substituent is halogen, —C(O)OH, or —C(O)OC₁₋₆ alkyl.

In some embodiments, R⁶ is selected from optionally substituted C₁₋₁₀ alkyl, optionally substituted C₂₋₁₀ alkenyl, and optionally substituted C₂₋₁₀ alkynyl.

In some embodiments, R⁶ is an optionally substituted C₁₋₁₀ alkyl. In some embodiments, the C₁₋₁₀ alkyl is a branched C₁₋₁₀ alkyl.

In some embodiments, R⁶ is an optionally substituted C₂₋₁₀ alkenyl. In some embodiments, the C₂₋₁₀ alkenyl is a branched C₂₋₁₀ alkenyl.

In some embodiments, R⁶ is an optionally substituted C₂₋₁₀ alkynyl. In some embodiments, the C₂₋₁₀ alkynyl is a branched C₂₋₁₀ alkynyl.

In some embodiments, R⁶ groups described above, selected from optionally substituted C₁₋₁₀ alkyl, optionally substituted C₂₋₁₀ alkenyl, and optionally substituted C₂₋₁₀ alkynyl, are independently substituted with one or more substituents selected from the group comprising optionally substituted C₁₋₆ alkyl, optionally substituted C₅₋₆ aryl, optionally substituted 5-6 membered heteroaryl, optionally substituted 5-6 membered heterocyclyl, halogen, —CN, —OR¹¹, —SR¹¹, —S(O)₂R¹¹, —S(O)₂N(R¹¹)₂, —S(O)(NR¹⁰)R¹¹, C(O) N(R¹¹)₂, —NR¹¹C(O)R¹¹, —N(R¹¹) S(O)₂R¹¹, —N(R¹¹)₂, —NO₂, —(O), ═(S), ═(N)R¹¹, —C(O)R¹¹, —C(O)OR¹¹, —C(O)N(R¹¹)₂, and —C(O)SR¹¹. In some embodiments, the one or more substituents are selected from the group consisting of —OR¹¹, —SR¹¹, —N(R¹¹)₂, —C(O)R¹¹, —C(O)OR¹¹, —C(O)N(R¹¹)₂, and —C(O)SR¹¹.

In some embodiments, the present disclosure provides a compound selected from the following structures:

| Compound | |
|---|---|
| number | Structure |
| 1 | 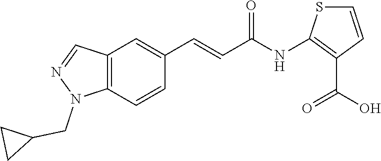 [SMILES: O=C(/C=C/c1ccc2c(cnn2CC2CC2)c1)Nc1sccc1C(=O)O] |
| 2 | 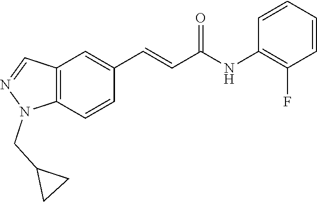 [SMILES: O=C(/C=C/c1ccc2c(cnn2CC2CC2)c1)Nc1ccccc1F] |
| 3 | 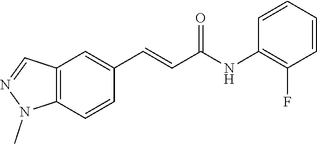 [SMILES: Cn1ncc2cc(/C=C/C(=O)Nc3ccccc3F)ccc21] |
| 4 | 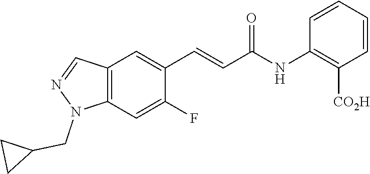 [SMILES: O=C(/C=C/c1cc2cnn(CC3CC3)c2cc1F)Nc1ccccc1C(=O)O] |
| 5 | 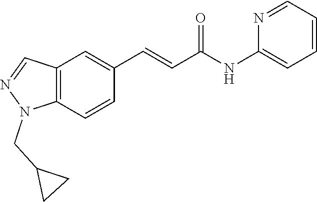 [SMILES: O=C(/C=C/c1ccc2c(cnn2CC2CC2)c1)Nc1ccccn1] |
| 6 | 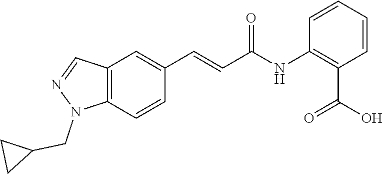 [SMILES: O=C(/C=C/c1ccc2c(cnn2CC2CC2)c1)Nc1ccccc1C(=O)O] |
| 7 | 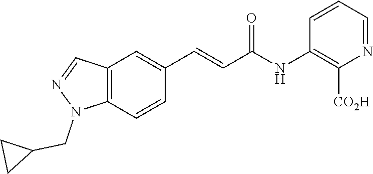 [SMILES: O=C(/C=C/c1ccc2c(cnn2CC2CC2)c1)Nc1cccnc1C(=O)O] |
| 8 | 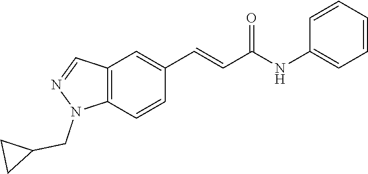 [SMILES: O=C(/C=C/c1ccc2c(cnn2CC2CC2)c1)Nc1ccccc1] |
| 9 | 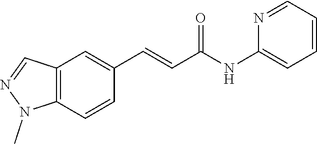 [SMILES: Cn1ncc2cc(/C=C/C(=O)Nc3ccccn3)ccc21] |
| 10 | 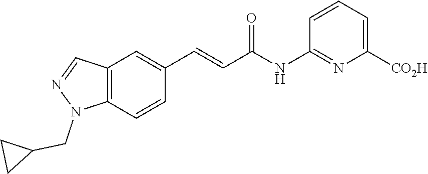 [SMILES: O=C(/C=C/c1ccc2c(cnn2CC2CC2)c1)Nc1cccc(C(=O)O)n1] |
| 11 | 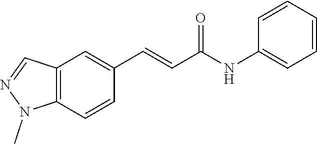 [SMILES: Cn1ncc2cc(/C=C/C(=O)Nc3ccccc3)ccc21] |
| 12 | 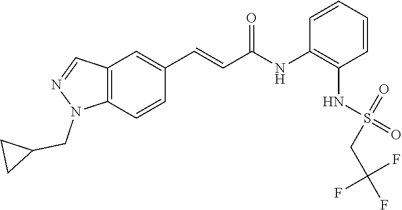 [SMILES: O=C(/C=C/c1ccc2c(cnn2CC2CC2)c1)Nc1ccccc1NS(=O)(=O)CC(F)(F)F] |
| 13 | 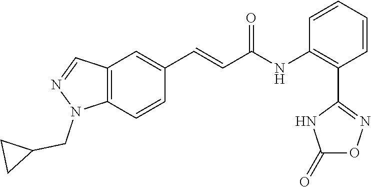 [SMILES: O=C(/C=C/c1ccc2c(cnn2CC2CC2)c1)Nc1ccccc1-c1noc(=O)[nH]1] |
| 14 | 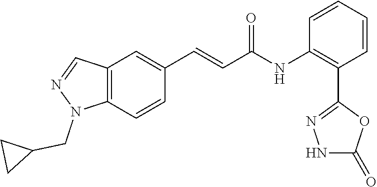 [SMILES: O=C(/C=C/c1ccc2c(cnn2CC2CC2)c1)Nc1ccccc1-c1n[nH]c(=O)o1] |
| 15 | 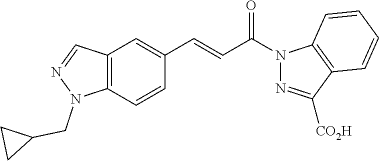 [SMILES: O=C(O)c1nn(C(=O)/C=C/c2ccc3c(cnn3CC3CC3)c2)c2ccccc12] |
| 16 | 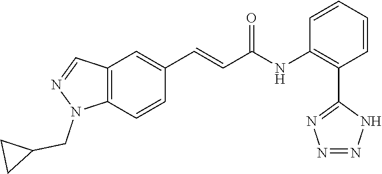 [SMILES: O=C(/C=C/c1ccc2c(cnn2CC2CC2)c1)Nc1ccccc1-c1nnn[nH]1] |
| 17 | 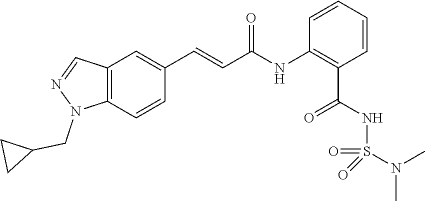 [SMILES: CN(C)S(=O)(=O)NC(=O)c1ccccc1NC(=O)/C=C/c1ccc2c(cnn2CC2CC2)c1] |
| 18 | 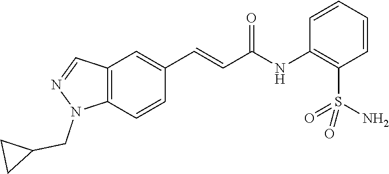 [SMILES: NS(=O)(=O)c1ccccc1NC(=O)/C=C/c1ccc2c(cnn2CC2CC2)c1] |
| 19 | 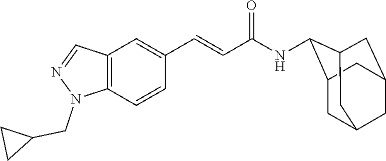 [SMILES: O=C(/C=C/c1ccc2c(cnn2CC2CC2)c1)NC1C2CC3CC(C2)CC1C3] |
| 20 | 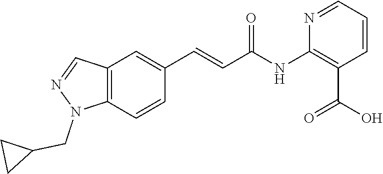 [SMILES: O=C(/C=C/c1ccc2c(cnn2CC2CC2)c1)Nc1ncccc1C(=O)O] |
| 21 | 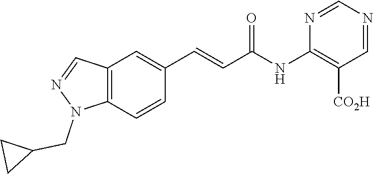 [SMILES: O=C(/C=C/c1ccc2c(cnn2CC2CC2)c1)Nc1ncncc1C(=O)O] |
| 22 | 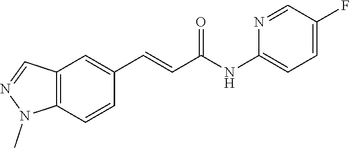 [SMILES: Cn1ncc2cc(/C=C/C(=O)Nc3ccc(F)cn3)ccc21] |
| 23 | 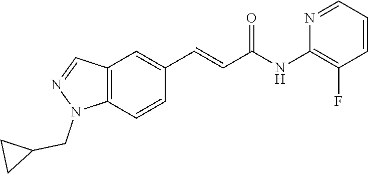 [SMILES: O=C(/C=C/c1ccc2c(cnn2CC2CC2)c1)Nc1ncccc1F] |
| 24 | 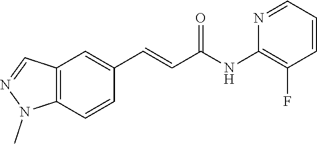 [SMILES: Cn1ncc2cc(/C=C/C(=O)Nc3ncccc3F)ccc21] |
| 25 | 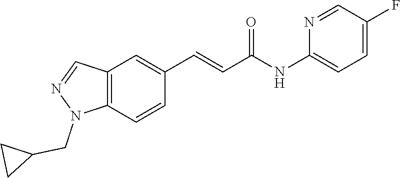 [SMILES: O=C(/C=C/c1ccc2c(cnn2CC2CC2)c1)Nc1ccc(F)cn1] |
| 26 | 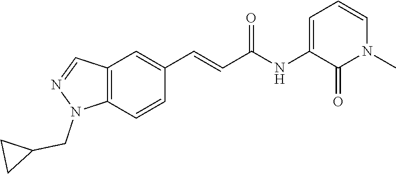 [SMILES: Cn1cccc(NC(=O)/C=C/c2ccc3c(cnn3CC3CC3)c2)c1=O] |
| 27 | 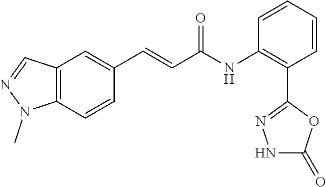 [SMILES: Cn1ncc2cc(/C=C/C(=O)Nc3ccccc3-c3n[nH]c(=O)o3)ccc21] |
| 28 | 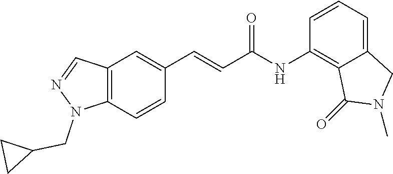 [SMILES: CN1Cc2cccc(NC(=O)/C=C/c3ccc4c(cnn4CC4CC4)c3)c2C1=O] |
| 29 | 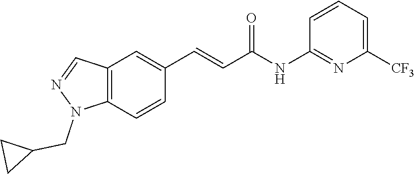 [SMILES: O=C(/C=C/c1ccc2c(cnn2CC2CC2)c1)Nc1cccc(C(F)(F)F)n1] |
| 30 | 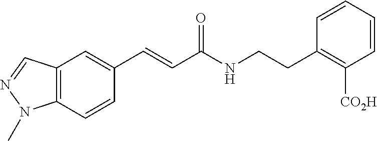 [SMILES: Cn1ncc2cc(/C=C/C(=O)NCCc3ccccc3C(=O)O)ccc21] |
| 31 | 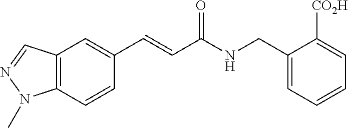 [SMILES: Cn1ncc2cc(/C=C/C(=O)NCc3ccccc3C(=O)O)ccc21] |
| 32 | 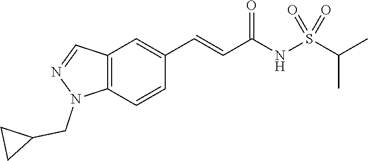 [SMILES: CC(C)S(=O)(=O)NC(=O)/C=C/c1ccc2c(cnn2CC2CC2)c1] |
| 33 | 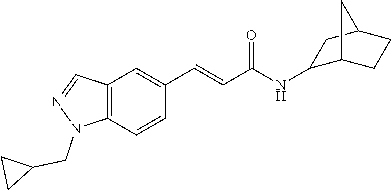 [SMILES: O=C(/C=C/c1ccc2c(cnn2CC2CC2)c1)NC1CC2CCC1C2] |
| 34 | 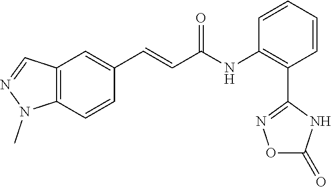 [SMILES: Cn1ncc2cc(/C=C/C(=O)Nc3ccccc3-c3noc(=O)[nH]3)ccc21] |
| 35 | 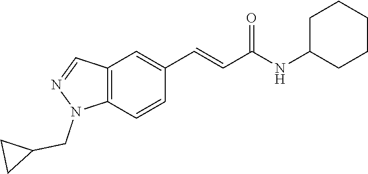 [SMILES: O=C(/C=C/c1ccc2c(cnn2CC2CC2)c1)NC1CCCCC1] |
| 36 | 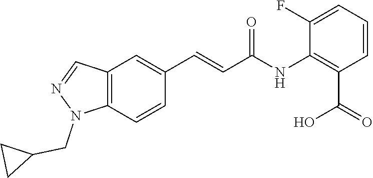 [SMILES: O=C(/C=C/c1ccc2c(cnn2CC2CC2)c1)Nc1c(F)cccc1C(=O)O] |
| 37 | 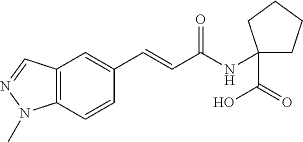 [SMILES: Cn1ncc2cc(/C=C/C(=O)NC3(C(=O)O)CCCC3)ccc21] |
| 38 | 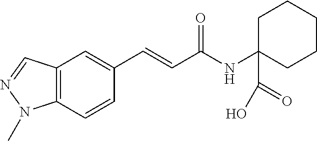 [SMILES: Cn1ncc2cc(/C=C/C(=O)NC3(C(=O)O)CCCCC3)ccc21] |
| 39 | 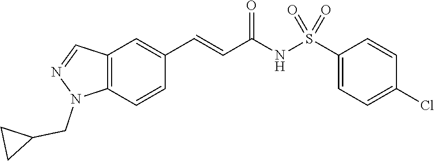 [SMILES: O=C(/C=C/c1ccc2c(cnn2CC2CC2)c1)NS(=O)(=O)c1ccc(Cl)cc1] |
| 40 | 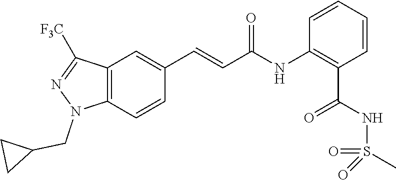 [SMILES: CS(=O)(=O)NC(=O)c1ccccc1NC(=O)/C=C/c1ccc2c(c1)c(C(F)(F)F)nn2CC1CC1] |
| 41 | 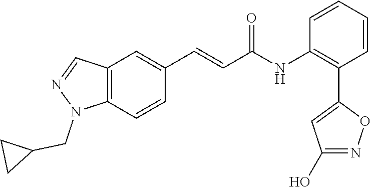 [SMILES: O=C(/C=C/c1ccc2c(cnn2CC2CC2)c1)Nc1ccccc1-c1cc(O)no1] |
| 42 | 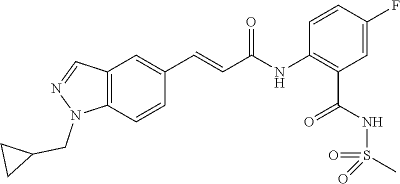 [SMILES: CS(=O)(=O)NC(=O)c1cc(F)ccc1NC(=O)/C=C/c1ccc2c(cnn2CC2CC2)c1] |
| 43 | 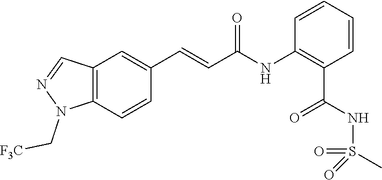 [SMILES: CS(=O)(=O)NC(=O)c1ccccc1NC(=O)/C=C/c1ccc2c(cnn2CC(F)(F)F)c1] |
| 44 | 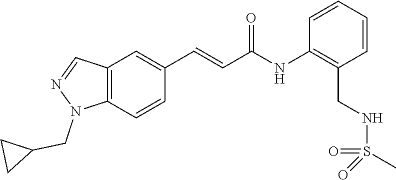 [SMILES: CS(=O)(=O)NCc1ccccc1NC(=O)/C=C/c1ccc2c(cnn2CC2CC2)c1] |
| 45 | 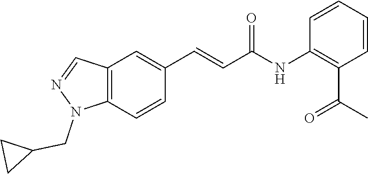 [SMILES: CC(=O)c1ccccc1NC(=O)/C=C/c1ccc2c(cnn2CC2CC2)c1] |
| 46 | 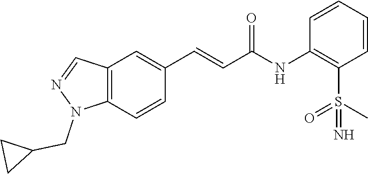 [SMILES: CS(=N)(=O)c1ccccc1NC(=O)/C=C/c1ccc2c(cnn2CC2CC2)c1] |
| 47 | 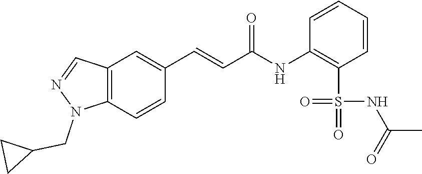 [SMILES: CC(=O)NS(=O)(=O)c1ccccc1NC(=O)/C=C/c1ccc2c(cnn2CC2CC2)c1] |
| 48 | 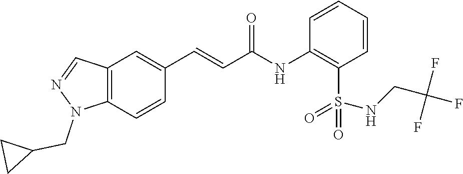 [SMILES: O=C(/C=C/c1ccc2c(cnn2CC2CC2)c1)Nc1ccccc1S(=O)(=O)NCC(F)(F)F] |
| 49 | 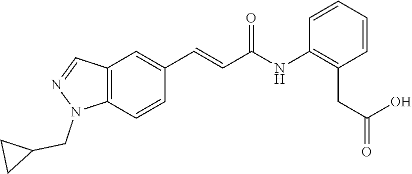 [SMILES: O=C(O)Cc1ccccc1NC(=O)/C=C/c1ccc2c(cnn2CC2CC2)c1] |
| 50 | 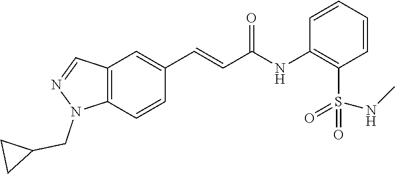 [SMILES: CNS(=O)(=O)c1ccccc1NC(=O)/C=C/c1ccc2c(cnn2CC2CC2)c1] |
| 51 | 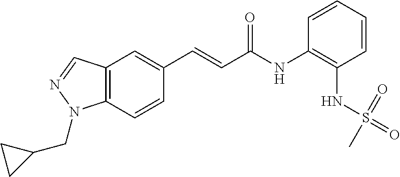 [SMILES: CS(=O)(=O)Nc1ccccc1NC(=O)/C=C/c1ccc2c(cnn2CC2CC2)c1] |
| 52 | 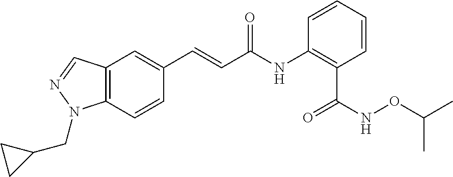 [SMILES: CC(C)ONC(=O)c1ccccc1NC(=O)/C=C/c1ccc2c(cnn2CC2CC2)c1] |
| 53 | 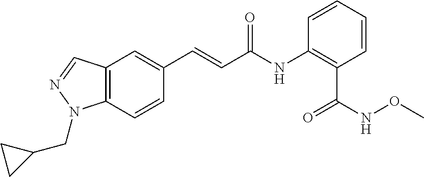 [SMILES: CONC(=O)c1ccccc1NC(=O)/C=C/c1ccc2c(cnn2CC2CC2)c1] |
| 54 | 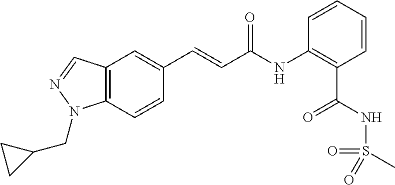 [SMILES: CS(=O)(=O)NC(=O)c1ccccc1NC(=O)/C=C/c1ccc2c(cnn2CC2CC2)c1] |
| 55 | 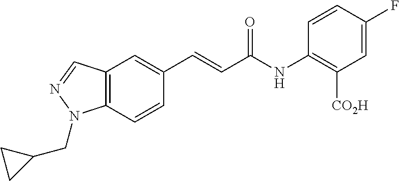 [SMILES: O=C(/C=C/c1ccc2c(cnn2CC2CC2)c1)Nc1ccc(F)cc1C(=O)O] |
| 56 | 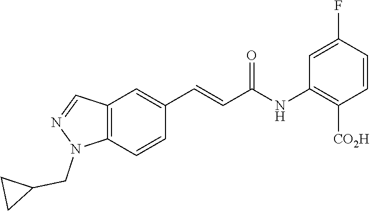 [SMILES: O=C(/C=C/c1ccc2c(cnn2CC2CC2)c1)Nc1cc(F)ccc1C(=O)O] |
| 57 | 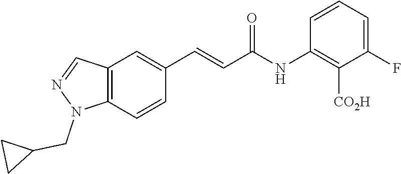 [SMILES: O=C(/C=C/c1ccc2c(cnn2CC2CC2)c1)Nc1cccc(F)c1C(=O)O] |
| 58 | 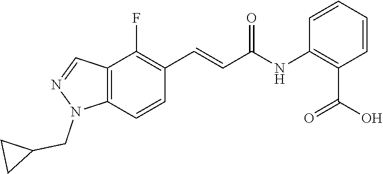 [SMILES: O=C(/C=C/c1ccc2c(cnn2CC2CC2)c1F)Nc1ccccc1C(=O)O] |
| 59 | 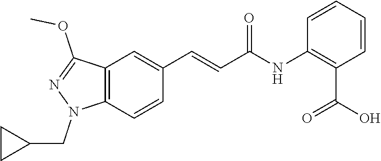 [SMILES: COc1nn(CC2CC2)c2ccc(/C=C/C(=O)Nc3ccccc3C(=O)O)cc12] |
| 60 | 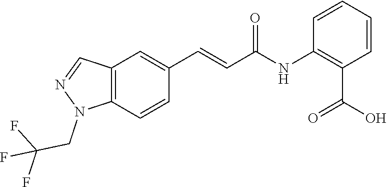 [SMILES: O=C(/C=C/c1ccc2c(cnn2CC(F)(F)F)c1)Nc1ccccc1C(=O)O] |
| 61 | 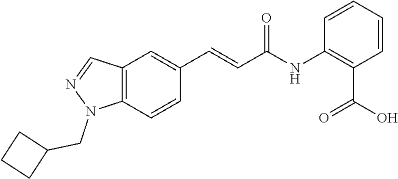 [SMILES: O=C(/C=C/c1ccc2c(cnn2CC2CCC2)c1)Nc1ccccc1C(=O)O] |
| 62 | 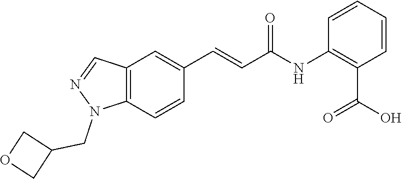 [SMILES: O=C(/C=C/c1ccc2c(cnn2CC2COC2)c1)Nc1ccccc1C(=O)O] |
| 63 | 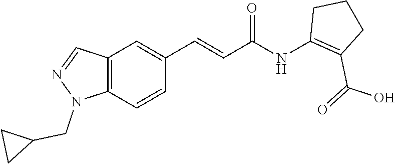 [SMILES: O=C(/C=C/c1ccc2c(cnn2CC2CC2)c1)NC1=C(C(=O)O)CCC1] |
| 64 | 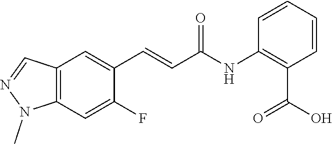 [SMILES: Cn1ncc2cc(/C=C/C(=O)Nc3ccccc3C(=O)O)c(F)cc21] |
| 65 | 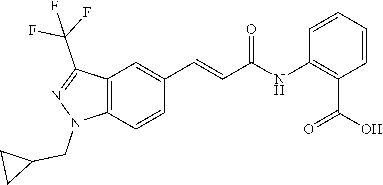 [SMILES: O=C(/C=C/c1ccc2c(c1)c(C(F)(F)F)nn2CC1CC1)Nc1ccccc1C(=O)O] |
| 66 | 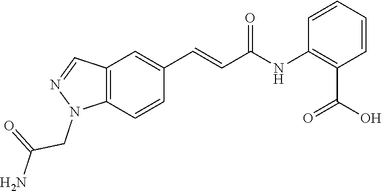 [SMILES: NC(=O)Cn1ncc2cc(/C=C/C(=O)Nc3ccccc3C(=O)O)ccc21] |
| 67 | 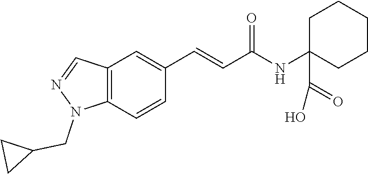 [SMILES: O=C(/C=C/c1ccc2c(cnn2CC2CC2)c1)NC1(C(=O)O)CCCCC1] |
| 68 | 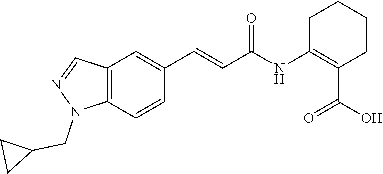 [SMILES: O=C(/C=C/c1ccc2c(cnn2CC2CC2)c1)NC1=C(C(=O)O)CCCC1] |
| 69 | 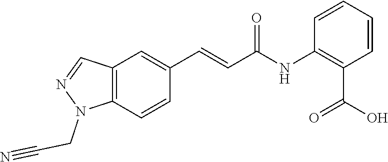 [SMILES: N#CCn1ncc2cc(/C=C/C(=O)Nc3ccccc3C(=O)O)ccc21] |
| 70 | 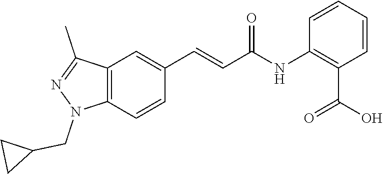 [SMILES: Cc1nn(CC2CC2)c2ccc(/C=C/C(=O)Nc3ccccc3C(=O)O)cc12] |
| 71 | 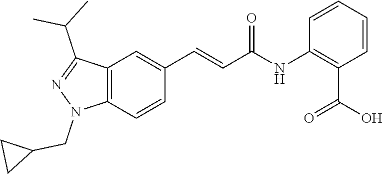 [SMILES: CC(C)c1nn(CC2CC2)c2ccc(/C=C/C(=O)Nc3ccccc3C(=O)O)cc12] |
| 72 | 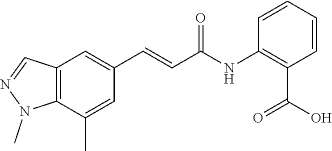 [SMILES: Cc1cc(/C=C/C(=O)Nc2ccccc2C(=O)O)cc2cnn(C)c12] |
| 73 | 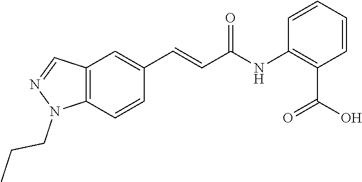 [SMILES: CCCn1ncc2cc(/C=C/C(=O)Nc3ccccc3C(=O)O)ccc21] |
| 74 | 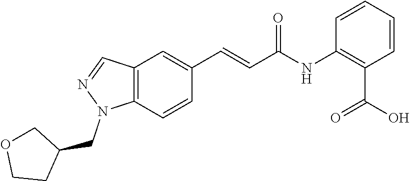 [SMILES: O=C(/C=C/c1ccc2c(cnn2C[C@H]2CCOC2)c1)Nc1ccccc1C(=O)O] |
| 75 | 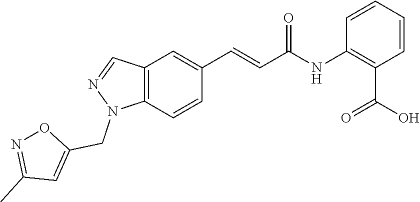 [SMILES: Cc1cc(Cn2ncc3cc(/C=C/C(=O)Nc4ccccc4C(=O)O)ccc32)on1] |
| 76 | 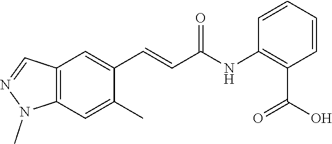 [SMILES: Cc1cc2c(cnn2C)cc1/C=C/C(=O)Nc1ccccc1C(=O)O] |
| 77 | 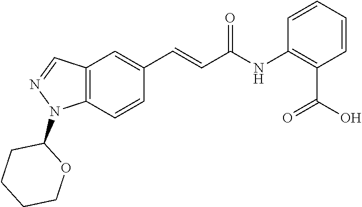 [SMILES: O=C(/C=C/c1ccc2c(cnn2[C@@H]2CCCCO2)c1)Nc1ccccc1C(=O)O] |
| 78 | 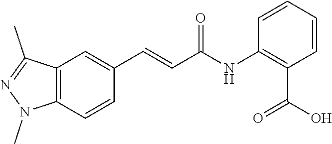 [SMILES: Cc1nn(C)c2ccc(/C=C/C(=O)Nc3ccccc3C(=O)O)cc12] |
| 79 | 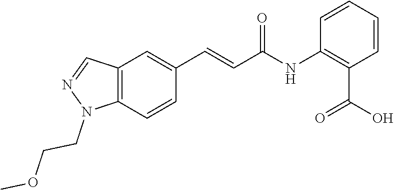 [SMILES: COCCn1ncc2cc(/C=C/C(=O)Nc3ccccc3C(=O)O)ccc21] |
| 80 | 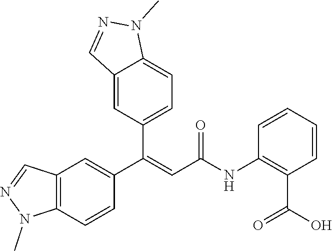 [SMILES: Cn1ncc2cc(C(=CC(=O)Nc3ccccc3C(=O)O)c3ccc4c(cnn4C)c3)ccc21] |
| 81 | 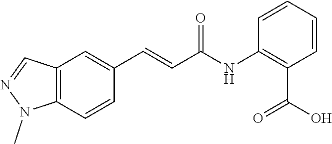 [SMILES: Cn1ncc2cc(/C=C/C(=O)Nc3ccccc3C(=O)O)ccc21] |

or a pharmaceutically acceptable salt, solvate or stereoisomer thereof.

In some embodiments, the compound of the present disclosure is selected from any of compounds 1-19, 23, 25, 36, 38, 54, 56, 62 and 68, or a pharmaceutically acceptable salt, solvate or stereoisomer thereof. In some embodiments, the compound of the present disclosure is selected from any of compounds 1-14, 16-18, 23, 25, 36, 54, 56 and 62, or a pharmaceutically acceptable salt, solvate or stereoisomer thereof. In some embodiments, the compound of the present disclosure is selected from any of compounds 1-14 and 56, or a pharmaceutically acceptable salt, solvate or stereoisomer thereof. In some embodiments, the compound of the present disclosure is selected from any of compounds 1-6, or a pharmaceutically acceptable salt, solvate or stereoisomer thereof. In some embodiments, the compound of the present disclosure is compound 6, or a pharmaceutically acceptable salt, solvate or stereoisomer thereof. In some embodiments, the compound of the present disclosure is selected from any of compounds 19, 38 and 68, or a pharmaceutically acceptable salt, solvate or stereoisomer thereof. In some embodiments, the compound of the present disclosure is compound 15, or a pharmaceutically acceptable salt, solvate or stereoisomer thereof.

In some embodiments, the present disclosure provides a compound having the structure

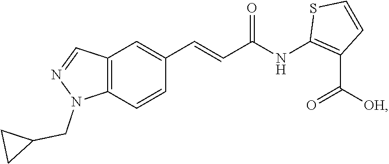 [SMILES: O=C(/C=C/c1ccc2c(cnn2CC2CC2)c1)Nc1sccc1C(=O)O]

or a pharmaceutically acceptable salt thereof. In certain embodiments of the compound or pharmaceutically acceptable salt, the compound is

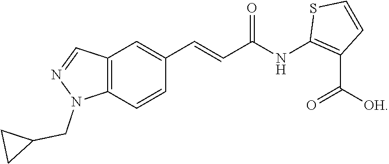 [SMILES: O=C(/C=C/c1ccc2c(cnn2CC2CC2)c1)Nc1sccc1C(=O)O]

In some embodiments, the present disclosure provides a compound having the structure

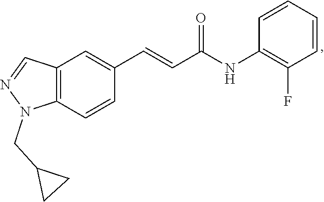 [SMILES: O=C(/C=C/c1ccc2c(cnn2CC2CC2)c1)Nc1ccccc1F]

or a pharmaceutically acceptable salt thereof. In certain embodiments of the compound or pharmaceutically acceptable salt, the compound is

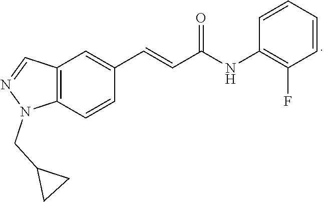 [SMILES: O=C(/C=C/c1ccc2c(cnn2CC2CC2)c1)Nc1ccccc1F]

In some embodiments, the present disclosure provides a compound having the structure

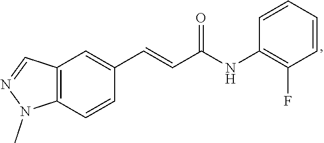 [SMILES: Cn1ncc2cc(/C=C/C(=O)Nc3ccccc3F)ccc21]

or a pharmaceutically acceptable salt thereof. In certain embodiments of the compound or pharmaceutically acceptable salt, the compound is

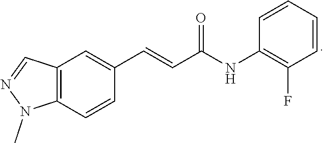 [SMILES: Cn1ncc2cc(/C=C/C(=O)Nc3ccccc3F)ccc21]

In some embodiments, the present disclosure provides a compound having the structure

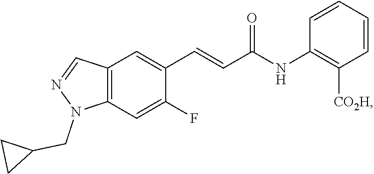 [SMILES: O=C(/C=C/c1cc2cnn(CC3CC3)c2cc1F)Nc1ccccc1C(=O)O]

or a pharmaceutically acceptable salt thereof. In certain embodiments of the compound or pharmaceutically acceptable salt, the compound is

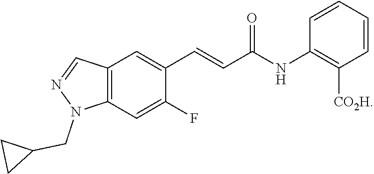 [SMILES: O=C(/C=C/c1cc2cnn(CC3CC3)c2cc1F)Nc1ccccc1C(=O)O]

In some embodiments, the present disclosure provides a compound having the structure

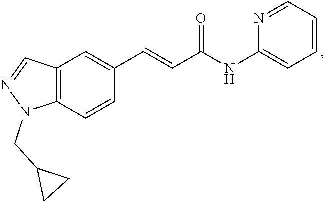 [SMILES: O=C(/C=C/c1ccc2c(cnn2CC2CC2)c1)Nc1ccccn1]

or a pharmaceutically acceptable salt thereof. In certain embodiments of the compound or pharmaceutically acceptable salt, the compound is

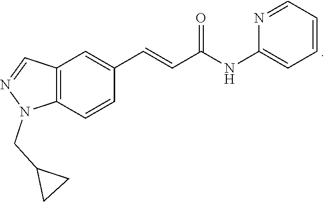 [SMILES: O=C(/C=C/c1ccc2c(cnn2CC2CC2)c1)Nc1ccccn1]

In some embodiments, the present disclosure provides a compound having the structure

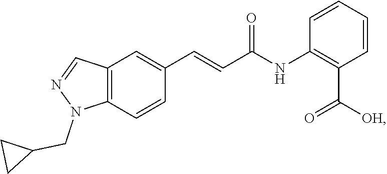 [SMILES: O=C(/C=C/c1ccc2c(cnn2CC2CC2)c1)Nc1ccccc1C(=O)O]

or a pharmaceutically acceptable salt thereof. In certain embodiments of the compound or pharmaceutically acceptable salt, the compound is

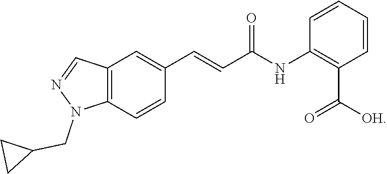 [SMILES: O=C(/C=C/c1ccc2c(cnn2CC2CC2)c1)Nc1ccccc1C(=O)O]

The compounds of the present disclosure may be provided in the form of a salt, preferably a pharmaceutically acceptable salt, solvate and/or stereoisomer thereof. In some embodiments, the compounds of the present disclosure may be provided in the form of a pharmaceutically acceptable salt thereof.

### Preparation of the Compounds

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

### Pharmaceutical and Compositions and Administration

While it is possible for compounds of the present disclosure to be administered as a raw chemical, it is also possible to present them as a pharmaceutical formulation.

Accordingly, the present disclosure provides a pharmaceutical composition comprising a compound or a pharmaceutically acceptable salt, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients can be used as suitable and as understood in the art; e.g., in Remington's Pharmaceutical Sciences. The pharmaceutical compositions of the present disclosure can be manufactured in a manner that is itself known, e.g., by means of conventional mixing, dissolving, granulating, dragee-making, levigating, emulsifying, encapsulating, entrapping or compression processes, for example.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route depends upon for example the condition and disorder of the recipient. The formulations can conveniently be presented in unit dosage form and can be prepared by any of the methods well known in the art. All methods include the step of bringing into association a compound of the present disclosure or a pharmaceutically acceptable salt, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present disclosure suitable for oral administration can be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient can also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets can be made by compression or molding, optionally with one or more accessory ingredients. Compressed tablets can be prepared by compressing in a suitable machine the active ingredient in a free-flowing form such as a powder or granules, optionally mixed with binders, inert diluents, or lubricating, surface active or dispersing agents. Molded tablets can be made by molding in a suitable machine a mixture of the powdered compound moistened with an inert liquid diluent. The tablets can optionally be coated or scored and can be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. The formulations can be presented in unit-dose or multi-dose containers, for example sealed ampoules and vials, and can be stored in powder form or in a freeze-dried (lyophilized) condition requiring only the addition of the sterile liquid carrier, for example, saline or sterile pyrogen-free water, immediately prior to use. Extemporaneous injection solutions and suspensions can be prepared from sterile powders, granules and tablets of the kind previously described.

Formulations for parenteral administration include aqueous and non-aqueous (oily) sterile injection solutions of the active compounds which can contain antioxidants, buffers, bacteriostats and solutes which render the formulation isotonic with the blood of the intended recipient; and aqueous and non-aqueous sterile suspensions which can include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions.

In addition to the formulations described above, the compounds of the present disclosure can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compounds can also be formulated in rectal compositions such as suppositories or retention enemas, e.g., containing conventional suppository bases such as cocoa butter, polyethylene glycol, or other glycerides. The compounds can also be formulated in vaginal compositions as gels, suppositories, or as dendrimers conjugates.

Compounds of the present disclosure can be administered topically, that is by non-systemic administration. Formulations suitable for topical administration include liquid or semi-liquid preparations suitable for penetration through the skin such as gels, liniments, lotions, creams, ointments or pastes.

Gels for topical or transdermal administration of compounds of the present disclosure can include a mixture of volatile solvents, nonvolatile solvents, and water. The volatile solvent component of the buffered solvent system can preferably include lower (C1-C6)alkyl alcohols, lower alkyl glycols and lower glycol polymers. More preferably, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. Preferably, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound can crystallize due to evaporation of volatile solvent, while an excess will result in a lack of bioavailability due to poor release of drug from solvent mixture. The buffer component of the buffered solvent system can be selected from any buffer commonly used in the art; preferably, water is used. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions or liniments for application to the skin can also include an agent to hasten drying and to cool the skin, such as an alcohol or acetone, and/or a moisturizer such as glycerol or an oil such as castor oil or arachis oil.

Creams, ointments or pastes according to the present disclosure are semi-solid formulations of the active ingredient for external application. They can be made by mixing the active ingredient in finely-divided or powdered form, alone or in solution or suspension in an aqueous or non-aqueous fluid, with the aid of suitable machinery, with a greasy or non-greasy base. The base can comprise hydrocarbons such as hard, soft or liquid paraffin, glycerol, beeswax, a metallic soap; a mucilage; an oil of natural origin such as almond, com, arachis, castor or olive oil; wool fat or its derivatives or a fatty acid such as steric or oleic acid together with an alcohol such as propylene glycol or a macrogel. The formulation can incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, can also be included.

### Methods of Use

Without being bound by theory, the compounds of the present disclosure were found to modulate, optionally to inhibit, proton-activated GPCRs such as GPR68 (OGR1).

Accordingly, in one aspect, the present disclosure provides a method of treating and/or preventing a disease, disorder or condition mediated by a proton-activated GPCR in a subject in need thereof, comprising administering to the subject an effective amount of a compound disclosed herein, or a pharmaceutically acceptable salt, solvate or stereoisomer thereof, or a pharmaceutical composition as disclosed herein, to the subject. The methods may include identifying a subject at risk for or having a disease, disorder or condition mediated by a proton-activated GPCR, and administering a compound to the subject in an effective amount for therapeutic treatment or prophylactic treatment.

In some embodiments, the proton-activated GPCR is GPR68 (OGR1). In other embodiments, the proton-activated GPCR is selected from GPR68 (OGR1), GPR4 (GPR4), GPR65 (TDAG8), and GPR132 (G2A).

The proton-activated GPCRs, such as GPR68 (OGR1), GPR4 (GPR4) and GPR65 (TDAG8), are involved in modulating key processes in inflammation, tumour biology, and fibrosis.

Accordingly, in some embodiments, the disease, disorder or condition mediated by a proton-activated GPCR is an inflammatory, fibrotic, or proliferative disease, disorder or condition. In certain embodiments, the disease, disorder or condition is a cancer.

In some embodiments, the compounds and methods provided herein are useful in the treatment of an inflammatory disease, disorder or condition.

Generally, inflammatory diseases, disorders or conditions treatable with the compounds disclosed herein relate to any diseases, disorders or condition characterized by an abnormal, irregular, excessive, rogue or unwarranted inflammatory response.

In some embodiment, the inflammatory disease, disorder or condition is the result of an injury to a tissue. In some embodiments, the inflammatory disease, disorder or condition is the result of an autoimmune condition, while in further embodiment, the inflammatory disease, disorder or condition is the result of a bacterial or viral infection or the presence of a toxin.

The compounds provided herein can have anti-inflammatory activity and/or immunomodulatory activity and can be useful in the treatment of diseases including but not limited to septic shock, haemodynamic shock, sepsis syndrome, post ischaemic reperfusion injury, malaria, mycobacterial infection, meningitis, psoriasis, congestive heart failure, fibrotic diseases, cachexia, graft rejection, cancers such as cutaneous T-cell lymphoma, diseases involving angiogenesis, autoimmune diseases, skin inflammatory diseases, inflammatory bowel diseases such as Crohn's disease and colitis, ankylosing spondylitis, psoriatic arthritis, adult Still's disease, ureitis, Wegener's granulomatosis, Behcehe disease, Sjogren's syndrome, sarcoidosis, polymyositis, dermatomyositis, multiple sclerosis, sciatica, complex regional pain syndrome, radiation damage, hyperoxic alveolar injury, periodontal disease, HIV, non-insulin dependent diabetes mellitus, systemic lupus erythematosus, glaucoma, sarcoidosis, idiopathic pulmonary fibrosis, bronchopulmonary dysplasia, retinal disease, scleroderma, osteoporosis, renal ischemia, myocardial infarction, cerebral stroke, cerebral ischemia, nephritis, hepatitis, glomerulonephritis, cryptogenic fibrosing aveolitis, psoriasis, transplant rejection, atopic dermatitis, vasculitis, allergy, seasonal allergic rhinitis, reversible airway obstruction, adult respiratory distress syndrome, asthma, chronic obstructive pulmonary disease (COPD) and/or bronchitis. The compounds and methods provided herein may be useful in treating one or more of these diseases, disorders or conditions.

In some embodiments, the inflammatory disease or disorder or condition is at least one inflammatory disease or disorder or condition selected from the group consisting of inflammatory bowel disease, celiac disease, colitis, irritable bowel syndrome, intestinal hyperplasia, metabolic syndrome, obesity, diabetes, rheumatoid arthritis, liver disease, hepatic steatosis, fatty liver disease, non-alcoholic fatty liver disease (NAFLD), and nonalcoholic steatohepatitis (NASH).

In some embodiments, the inflammatory disease, disorder or condition is arthritis, osteoarthritis, psoriatic arthritis, rheumatoid arthritis, diabetic retinopathy, retinal inflammation, retinitis, Sjogren's syndrome, macular degeneration, gout, pseudogout, pericarditis, or uveitis.

In some embodiments, the inflammatory disease, disorder or condition is focal segmental glomerulosclerosis, lupus nephritis, membranous nephropathy, IgA nephropathy, chronic obstructive pulmonary disorder (COPD), asthma, or cystic fibrosis.

In some embodiments, the inflammatory disease, disorder or condition is an autoimmune disease, disorder or condition. In some embodiments, the autoimmune disease, disorder or condition treatable by the compounds and methods provided herein is systemic lupus erythematosus (SLE), inflammatory bowel disease, Crohn's disease, ulcerative colitis, graft versus host disease, multiple sclerosis, or rheumatoid arthritis.

In some embodiments, the compounds and methods provided herein are useful in the treatment of a fibrotic disease, disorder or condition.

A “fibrotic condition”, “fibrotic disease”, and “fibrotic disorder”, are used interchangeably to refer to a condition, disease or disorder that is characterized by dysregulated proliferation or activity of fibroblasts and/or abnormal accumulation of fibronectin and/or pathologic or excessive accumulation of collagenous tissue. Fibrotic disorders include, but are not limited to, renal fibrosis, dermal fibrosis, pancreatic fibrosis, liver fibrosis (e.g., hepatic fibrosis associated with chronic active hepatitis), and pulmonary fibrosis, including idiopathic pulmonary fibrosis (IPF) and pulmonary fibrosis from a known etiology.

In some embodiments, the fibrotic disease, disorder or condition is renal fibrosis, pulmonary fibrosis, cardiovascular fibrosis, ocular fibrosis, dermal fibrosis, pancreatic fibrosis, or liver fibrosis.

In some embodiments, the fibrotic diseases, disorders or conditions suitable for treatment by the methods provided herein include, but are not limited to, kidney disease, such as progressive glomerular kidney disease, glomerulonephritis, and diabetic nephropathy; lung disease, such as pulmonary fibrosis; fibrotic skin disorders, such as keloids, hypertrophic scars and scleroderma; heart disease, such as heart failure due to ischaemic heart disease, valvular heart disease and hypertensive heart disease, diabetic cardiomyopathy and hypertension; and liver disease, such as fibrosis of the liver.

In certain embodiments, the fibrotic disease, disorder or condition is kidney disease. In some embodiments, the kidney disease may include, but is not limited to, a progressive glomerular kidney disease including without limitation diabetic nephropathy (e.g., as a consequence of Type I or Type Il diabetes or systemic lupus), primary glomerulonephritis (e.g., membranous nephropathy, focal segmental glomerulosclerosis, membranoproliferative glomerulonephritis, diffuse proliferative glomerulonephritis, membranous focal segmental glomerulosclerosis) or secondary glomerulonephritis (e.g., diabetic nephropathy, ischemic nephropathy). In some embodiments, the kidney disease may include progressive kidney diseases with origins primarily in the tubulointerstitium. In some embodiments, the kidney disease may include, e.g., chronic interstitial nephritis, autosomal dominant tubulointerstitial fibrosis, or reflux nephropathy.

In some embodiments, the fibrotic diseases, disorders or conditions suitable for treatment by the methods provided herein include, but are not limited to, diabetic cardiomyopathy, congestive heart failure, ischemic heart disease, hypertension, peripheral artery disease, cerebrovascular disease, chronic kidney disease, diabetic nephropathy, systemic sclerosis (scleroderma), hypertrophic scars, keloids, idiopathic pulmonary fibrosis (IPF), non-alcoholic steatohepatitis (NASH), non-alcoholic fatty liver disease (NAFLD), primary biliary cirrhosis (PBC), and primary sclerosis cholangitis (PSC).

In some embodiments, the compounds and methods provided herein are useful in the treatment of a proliferative disease, disorder or condition.

Generally, proliferative diseases, disorders or conditions treatable with the compounds disclosed herein relate to any diseases, disorders or condition characterized by aberrant cell proliferation. Cell proliferation may be self-propagating and includes an inappropriate or excessive wound healing response. The cellular proliferation can increase the influx of inflammatory cytokines and inflammatory cells, and thus be associated with inflammation and/or degeneration.

Proliferative diseases, disorders or conditions treatable with the compounds disclosed herein include, but are not limited to, various retinopathies.

In some embodiments, the proliferative diseases, disorders or conditions suitable for treatment by the methods provided herein include, but are not limited to, diabetic retinopathy, diabetic macular edema, macular degeneration, retinopathy of prematurity (ROP), and proliferative vitreoretinopathy (PVR).

Proliferative diseases, disorders or conditions treatable with the compounds disclosed herein further include, but are not limited to, various tumours and cancers, benign or malignant, metastatic or non-metastatic.

Cancers treatable with the compounds disclosed herein include a variety of cancers, including, among others, breast cancer, skin cancer, ovarian cancer, renal cancer, gastrointestinal cancer, kidney cancer, bladder cancer, pancreatic cancer, lung squamous carcinoma, and adenocarcinoma.

In some embodiments, the cancer is a cancer that is characterized by the presence of one or more tumours in the subject. Examples of cancers suitable for treatment by the compounds and methods provided herein include, but are not limited to, metastatic melanoma, metastatic prostate cancer, metastatic breast cancer, triple negative breast cancer, bladder cancer, brain cancer, esophageal cancer, liver cancer, head and neck cancer, squamous cell lung cancer, non-small lung cell cancer, Merkel cell carcinoma, sarcoma, hepatocellular cancer, multiple myeloma, pancreatic cancer, colorectal carcinoma, cervical cancer, gastric carcinoma, kidney cancer, metastatic renal cell carcinoma, leukemia, ovarian cancer, and malignant glioma. In certain embodiments, the cancer is metastatic melanoma, metastatic prostate cancer, or metastatic breast cancer. In some embodiments, the subject has received an allogeneic tissue graft associated with treatment for cancer, e g, after hematopoietic stem cell transplantation used for treatment of a leukemia.

In some embodiments, the cancer suitable for treatment by the compounds and methods provided herein is a breast cancer, skin cancer (melanoma), pancreatic cancer, lung cancer, prostate cancer, colon cancer, liver cancer (hepatocellular carcinoma), head and neck cancer, ovarian cancer, or kidney cancer.

In some embodiments, the subject is a human.

The term “therapeutically effective amount,” as used herein, refers to an amount of a compound sufficient to cure, ameliorate, slow progression of, prevent, or reduce the likelihood of onset of the identified disease or condition, or to exhibit a detectable therapeutic, prophylactic, or inhibitory effect. The effect can be detected by, for example, the assays disclosed in the following examples. The precise effective amount for a subject will depend upon the subject's body weight, size, and health; the nature and extent of the condition; and the therapeutic or combination of therapeutics selected for administration. Therapeutically and prophylactically effective amounts for a given situation can be determined by routine experimentation that is within the skill and judgment of the clinician.

For any compound, the therapeutically or prophylactically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans.

Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are preferred. However, pharmaceutical compositions that exhibit narrow therapeutic indices are also within the scope of the invention. The data obtained from cell culture assays and animal studies may be used in formulating a range of dosage for human use. The dosage contained in such compositions is preferably within a range of circulating concentrations that include an ED₅₀ with little or no toxicity. The dosage may vary within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

An effective amount of the compounds of the present disclosure for modulating, such as inhibiting, a proton-activated GPCR such as GPR68 (OGR1) includes an amount that can establish a concentration between about 1 nm and about 1,000 μM in one embodiment, between about 1 nM to about 250 μM in another embodiment, between about 1 nM to about 100 μM in another embodiment, between about 1 nM to about 50 μM in yet another embodiment. In some embodiments, an effective amount of the compounds of the present disclosure for modulating, such as inhibiting, a proton-activated GPCR such as GPR68 (OGR1) is an amount that can establish a concentration of about between about 1 μM to about 100 μM, about 1 μM to about 50 μM, or about 1 μM to about 20 μM. In some embodiments, an effective amount of the compounds of the present disclosure for modulating, such as inhibiting, a proton-activated GPCR such as GPR68 (OGR1) is an amount that can establish a concentration of about 100 μM, about 90 μM, about 80 μM, about 70 μM, about 60 μM, about 50 μM, about 40 μM, about 30 μM, about 20 μM, about 10 μM, or about 1 μM. In certain embodiments, an effective amount of the compounds of the present disclosure for modulating, such as inhibiting, a proton-activated GPCR such as GPR68 (OGR1) is an amount that can establish a concentration of about between about 1 μM to about 20 μM, about 1 μM to about 15 μM, about 1 μM to about 10 μM, or about 1 μM to about 5 μM. As will be clear to a skilled artisan, the exact amount of the compound can depend on a variety of factors including weight, age, sex. An effective amount of compounds of the present disclosure can include dosing regimens according to the pharmacokinetics/metabolism of the compounds and can also vary depending on the stage or severity of the disease, condition or disorder when the disease, condition or disorder is already established. Dosing and quantities are also influenced by mode of administration, such as oral versus intravenous administration.

In some embodiments, treating a disease, disorder or condition described herein results in an increase in average survival time of a population of treated subjects in comparison to a population of untreated subjects. Preferably, the average survival time is increased by more than about 30 days; more preferably, by more than about 60 days; more preferably, by more than about 90 days; and even more preferably by more than about 120 days. An increase in survival time of a population may be measured by any reproducible means. In a preferred aspect, an increase in average survival time of a population may be measured, for example, by calculating for a population the average length of survival following initiation of treatment with an active compound. In an another preferred aspect, an increase in average survival time of a population may also be measured, for example, by calculating for a population the average length of survival following completion of a first round of treatment with an active compound.

In some embodiments, treating a disease, disorder or condition described herein results in a decrease in the mortality rate of a population of treated subjects in comparison to a population of subjects receiving carrier alone. In another aspect, treating a condition described herein results in a decrease in the mortality rate of a population of treated subjects in comparison to an untreated population. In a further aspect, treating a condition described herein results a decrease in the mortality rate of a population of treated subjects in comparison to a population receiving monotherapy with a drug that is not a compound of the embodiments, or a pharmaceutically acceptable salt, metabolite, analog or derivative thereof. Preferably, the mortality rate is decreased by more than about 2%; more preferably, by more than about 5%; more preferably, by more than about 10%; and most preferably, by more than about 25%. In a preferred aspect, a decrease in the mortality rate of a population of treated subjects may be measured by any reproducible means. In another preferred aspect, a decrease in the mortality rate of a population may be measured, for example, by calculating for a population the average number of disease-related deaths per unit time following initiation of treatment with an active compound. In another preferred aspect, a decrease in the mortality rate of a population may also be measured, for example, by calculating for a population the average number of disease related deaths per unit time following completion of a first round of treatment with an active compound.

In certain embodiments, treating a disease, disorder or condition described herein results in a reduction in the rate of cellular proliferation. Preferably, after treatment, the rate of cellular proliferation is reduced by at least about 5%; more preferably, by at least about 10%; more preferably, by at least about 20%; more preferably, by at least about 30%; more preferably, by at least about 40%; more preferably, by at least about 50%; even more preferably, by at least about 60%; and most preferably, by at least about 75%. The rate of cellular proliferation may be measured by any reproducible means of measurement. In a preferred aspect, the rate of cellular proliferation is measured, for example, by measuring the number of dividing cells in a tissue sample per unit time.

In another aspect, treating a disease, disorder or condition described herein results in a reduction in the proportion of proliferating cells. Preferably, after treatment, the proportion of proliferating cells is reduced by at least about 5%; more preferably, by at least about 10%; more preferably, by at least about 20%; more preferably, by at least about 30%; more preferably, by at least about 40%; more preferably, by at least about 50%; even more preferably, by at least about 60%; and most preferably, by at least about 75%. The proportion of proliferating cells may be measured by any reproducible means of measurement. In a preferred aspect, the proportion of proliferating cells is measured, for example, by quantifying the number of dividing cells relative to the number of nondividing cells in a tissue sample. In another preferred aspect, the proportion of proliferating cells is equivalent to the mitotic index.

In another aspect, treating a disease, disorder or condition described herein results in a decrease in size of an area or zone of cellular proliferation. Preferably, after treatment, the size of an area or zone of cellular proliferation is reduced by at least 5% relative to its size prior to treatment; more preferably, reduced by at least about 10%; more preferably, reduced by at least about 20%; more preferably, reduced by at least about 30%; more preferably, reduced by at least about 40%; more preferably, reduced by at least about 50%; even more preferably, reduced by at least about 60%; and most preferably, reduced by at least about 75%. Size of an area or zone of cellular proliferation may be measured by any reproducible means of measurement. In a preferred aspect, size of an area or zone of cellular proliferation may be measured as a diameter or width of an area or zone of cellular proliferation.

The methods described herein may include identifying a subject in need of treatment. In a preferred embodiment, the methods include identifying a mammal in need of treatment. In a highly preferred embodiment, the methods include identifying a human in need of treatment. Identifying a subject in need of treatment may be accomplished by any means that indicates a subject who may benefit from treatment. For example, identifying a subject in need of treatment may occur by clinical diagnosis, laboratory testing, or any other means known to one of skill in the art, including any combination of means for identification.

As described elsewhere herein, the compounds described herein may be formulated in pharmaceutical compositions, if desired, and can be administered by any route that permits treatment of the disease or condition. A preferred route of administration is oral administration. Administration may take the form of single dose administration, or the compound of the embodiments can be administered over a period of time, either in divided doses or in a continuous-release formulation or administration method (e.g., a pump). However the compounds of the embodiments are administered to the subject, the amounts of compound administered and the route of administration chosen should be selected to permit efficacious treatment of the disease condition.

Further embodiments include administering a combination of compounds to a subject in need thereof. A combination can include a compound, composition, pharmaceutical composition described herein with an additional medicament for treating and/or preventing a disease, disorder or condition mediated by a proton-activated GPCR.

Some embodiments include co-administering a compound, composition, and/or pharmaceutical composition described herein, with an additional medicament. By “co-administration,” it is meant that the two or more agents may be found in the patient's bloodstream at the same time, regardless of when or how they are actually administered. In some embodiments, the agents are administered simultaneously. In some such embodiments, administration in combination is accomplished by combining the agents in a single dosage form. In some embodiments, the agents are administered sequentially. In some embodiments the agents are administered through the same route, such as orally. In some other embodiments, the agents are administered through different routes, such as one being administered orally and another being administered by i.v. Thus, for example, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods described herein may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

For example, the compounds and/or pharmaceutical compositions described herein may be co-administered with an additional medicament or therapy for treating and/or preventing a disease, disorder or condition mediated by a proton-activated GPCR. In some embodiments, the compounds and/or pharmaceutical compositions described herein may be co-administered with a medicament or therapy for the treatment and/or prevention of an inflammatory, fibrotic, or proliferative disease, disorder or condition. In certain embodiments, the compounds and/or pharmaceutical compositions described herein may be co-administered with a medicament or therapy for the treatment and/or prevention of a cancer.

Further embodiments of the present disclosure provide a method of inhibiting a proton-activated GPCR in a cell, comprising administering to the cell an effective amount of compounds of the present disclosure. The cell may be in a cell culture. The cell may be present in or otherwise comprise a tissue. The tissue may be a human tissue.

In some embodiments, the proton-activated GPCR is a GPR68 (OGR1).

In some embodiments, the cell is a tumour cell. The tumour cell may be a tumour cell of the pancreas, a tumour cell of the head and neck, a tumour cell of the lung, a tumour cell of the kidney, a tumour cell of the breast, a tumour cell of the colon, a tumour cell of the ovary, a tumour cell of a lymph node, a tumour cell of the stomach, a tumour cell of the esophagus, a tumour cell of the skin, a tumour cell of the brain, a tumour cell of the oral cavity, a tumour cell of the pharynx, a tumour cell of the thyroid, a tumour of the adrenal gland, a leukemia cell, a sarcoma cell, a tumour cell of the testes, a tumour cell of the bladder, or a tumour cell of the prostate gland. The tumour cell may be in a cell culture. The cell may be present in or otherwise comprise a tumour tissue.

The present disclosure is further described by the following examples, which are not intended to limit the scope of the claims.

### Example 1. Experimental Preparative and Analytical Conditions

### Abbreviations

Chloroform-d (deuterated chloroform); DMSO-d₆ (deuterated dimethylsulfoxide); Methanol-d₄ (deuterated methanol); DMF (N,N-dimethylformamide); DCM (dichloromethane); PE (petroleum ether); ESI (electrospray atmospheric pressure ionization); TEA (triethylamine); TFA (trifluoroacetic acid); dioxane (1,4-dioxane); THF (tetrahydrofuran); EtOH (ethanol); H₂O (water); MeCN (Acetonitrile); EtOAc (ethyl acetate); g (gram); h (hour); nm (nanometer); ¹H NMR (proton nuclear magnetic resonance); Hz (hertz); LC-MS (liquid chromatography-mass spectrometry); MS (mass spectrometry); mg (milligrams); MHz (megahertz); min (minutes); mL (millilitres), mmol (millimoles); ppm (parts per million); Rt (retention time); RT (room temperature); TLC (thin layer chromatography); v/v (volume/volume); m/z (mass charge ratio); HOAc (acetic acid); (CH₃)₃SnOH (Trimethyltin hydroxide); PPSE (Trimethylsilyl polyphosphate); DCE (1,2-Dichloroethane); HATU (O-(7-Azabenzotriazol-1-yl)-N,N,N′,N′-tetraMethyluroniuM hexafluorophosphate); (COCl)₂ (Oxalyl chloride); Cs₂CO₃ (Cesium carbonate); NH₄Cl (ammonium chloride); SOCl₂ (Thionyl chloride); DIPEA (N,N-diisopropylethylamine); MW (microwave); NH₄Cl (Ammonium chloride); DMAP (4-Dimethylaminopyridine); LiOH (Lithium hydroxide); NaH (Sodium hydride); Aq (aqueous); HPLC: high performance liquid chromatography; M: molar, molecular ion; UV: ultraviolet; UPLC: ultra performance liquid chromatography.

### General Experimental

All starting materials and solvents were obtained either from commercial sources or prepared according to the literature citation. Reaction mixtures were magnetically stirred and reactions performed at room temperature (ca. 20° C.) unless otherwise indicated.

Column chromatography was performed on an automated flash chromatography system, such as a Biotage Isolera Rf system, using pre-packed silica (40 μm) cartridges, unless otherwise indicated.

¹H NMR spectra were recorded using a Bruker AVANCE 400 MHz spectrometer. Data for 1H are reported as chemical shift (ppm) and multiplicity (s=singlet, d=doublet, t=triplet, q=quartet, m=multiplet). Chemical shifts are expressed in parts per million using either the central peaks of the residual protic solvent or an internal standard of tetramethylsilane as references. The spectra were recorded at 298K unless otherwise indicated.

Analytical UPLC-MS experiments to determine retention times and associated mass ions were performed using a Waters ACQUITY UPLC® H-Class system, equipped with ACQUITY PDA Detector and ACQUITY QDa Mass Detector, running one of the analytical methods described below.

Analytical LC-MS experiments to determine retention times and associated mass ions were performed using an Agilent 1200 series HPLC system coupled to an Agilent 1956, 6100 or 6120 series single quadrupole mass spectrometer running one of the analytical methods described below.

### Preparative HPLC Generic Methods:

HPLC Instruments: Shimadzu 20AP UV detector: SPD-20A. UV wavelength: 214 nm and 254 nm.

Conditions 1: Mobile phase A: water; Mobile phase B: acetonitrile.

Conditions 2: Mobile phase A: water with 0.1% trifluoroacetic acid; Mobile phase B: acetonitrile.

Conditions 3: Mobile phase A: water with 0.1% formic acid; Mobile phase B: acetonitrile.

Conditions 4: Mobile phase A: water with 0.1% ammonium hydroxide; Mobile phase B: acetonitrile.

Column: Agilent 10 Prep-C18 250×21.2 mm. Column temperature: Ambient

LC gradient: 20% to 85% in 20 min; then 85% to 100% in 0.01 min; then hold 100% for 5 min; then 100% to 20% in 0.01 min; hold at 20% for 5 min.

LC Flow rate: 20 mL/min binary pump.

Nomenclature of structures was generated using ‘Structure to Name’ conversion from ChemDraw® Professional 17 (PerkinElmer).

### Analytical Methods

### Method 1—Acidic Method (Shimadzu 3 Min)

Column: Shimadzu LC-20AD series, Binary Pump, Diode Array Detector. Agilent Poroshell 120 EC-C18, 2.7 μm, 4.6×50 mm column.

Detection: 2020, Quadrupole LC/MS, Ion Source: API-ESI, TIC: 100~900 m/z, Drying gas flow: 15 L/min, Nebulizer pressure: 1.5 L/min, Drying gas temperature: 250° C., Vcap: 4500V. Samples were dissolved in methanol at 1~10 μg/mL, then filtered through a 0.22 μm filter membrane. Injection volume: 1~10 μL. Detector: 214 nm, 254 nm. Detection wavelength: 214 nm, 254 nm.

Solvents: A: 0.05% v/v Formic acid in water, B: 0.05% v/v Formic acid in MeCN.

### Gradient:

| | | | Flow rate |
|---|---|---|---|
| T (min) | A(%) | B(%) | (mL/min) |
| 0.00 | 80 | 15 | 1.5 |
| 0.28 | 80 | 15 | 1.5 |
| 2.38 | 10 | 90 | 1.5 |
| 2.39 | 0 | 100 | 1.5 |
| 2.69 | 0 | 100 | 1.5 |
| 2.70 | 85 | 15 | 1.5 |
| 3.00 | 85 | 15 | 1.5 |

### Method 2—Acidic 5 Min Method (Shimadzu 5 Min)

Column: Shimadzu LC-20AD series, Binary Pump, Diode Array Detector. Agilent Poroshell 120 EC-C18, 2.7 μm, 4.6×50 mm column.

Detection: 2020, Quadrupole LC/MS, Ion Source: API-ESI, TIC: 100~900 m/z, Drying gas flow: 15 L/min, Nebulizer pressure: 1.5 L/min, Drying gas temperature: 250° C., Vcap: 4500V. Samples were dissolved in methanol at 1~10 μg/mL, then filtered through a 0.22 μm filter membrane. Injection volume: 1~10 μL. Detection wavelength: 214 nm, 254 nm.

Solvents: A: 0.05% formic acid in water (v/v), B: 0.05% formic acid in MeCN (v/v).

### Gradient:

| | | | Flow rate |
|---|---|---|---|
| T (min) | A(%) | B(%) | (mL/min) |
| 0.00 | 80 | 15 | 1.0 |
| 0.50 | 80 | 15 | 1.0 |
| 4.00 | 15 | 85 | 1.0 |
| 4.01 | 0 | 100 | 1.0 |
| 4.50 | 0 | 100 | 1.0 |
| 4.51 | 85 | 15 | 1.0 |
| 5.00 | 85 | 15 | 1.0 |

### Method 3—Acidic Method (Waters QDa 3 Min)

Column: Waters QDa, Binary Pump, Diode Array Detector. Waters CORTECS UPLC, C18, 1.6 μm, 2.1×50 mm column.

Detection: QDa, Quadrupole LC/MS, Ion Source: API-ES, TIC: 70~900 m/z, Fragmentor: 70, Drying gas flow: 12 L/min, Nebulizer pressure: 36 psi, Drying gas temperature: 350° C., Vcap: 3000V. Samples were dissolved in methanol at 1~10 μg/mL, then filtered through a 0.22 μm filter membrane. Injection volume: 1~10 μL. Detector: 214 nm, 254 nm.

Solvents: A: 0.05% Formate in water (v/v), B: 0.05% Formate in MeCN(v/v).

### Gradient:

| | | | Flow rate |
|---|---|---|---|
| T (min) | A(%) | B(%) | (mL/min) |
| 0.00 | 80 | 20 | 0.6 |
| 1.80 | 20 | 80 | 0.6 |
| 2.65 | 20 | 80 | 0.6 |
| 2.80 | 80 | 20 | 0.6 |
| 3.00 | 80 | 20 | 0.6 |

### Method 4—Acidic Method (Agilent 3 Min)

Column: Agilent Technologies 1290 series, Binary Pump, Diode Array Detector. Agilent EclipsePlus RRHD C18, 1.8 μm, 3.0×50 mm.

Detection: G6120A, Quadrupole LC/MS, Ion Source: API-ES, TIC: 70~1000 m/z, Fragmentor: 70, Drying gas flow: 12 L/min, Nebulizer pressure: 36 psi, Drying gas temperature: 350° C., Vcap: 3000V. Samples were dissolved in methanol at 1~10 μg/mL, then filtered through a 0.22 μm filter membrane. Injection volume: 1~10 μL. Detector: 214 nm, 254 nm.

Solvents: A: 0.05% Formate in water (v/v), B: 0.05% Formate in MeCN(v/v).

### Gradient:

| | | | Flow rate |
|---|---|---|---|
| T (min) | A(%) | B(%) | (mL/min) |
| 0.00 | 80 | 20 | 0.8 |
| 2.65 | 15 | 85 | 0.8 |
| 3.00 | 15 | 85 | 0.8 |

### Example 2. Synthesis of Intermediates

The compounds of the present disclosure may be prepared by methods well known to those skilled in the art, and/or as described in the synthetic experimental procedures shown below.

### Intermediate 1. (E)-3-(1-(cyclopropylmethyl)-1H-indazol-5-yl)acrylic acid

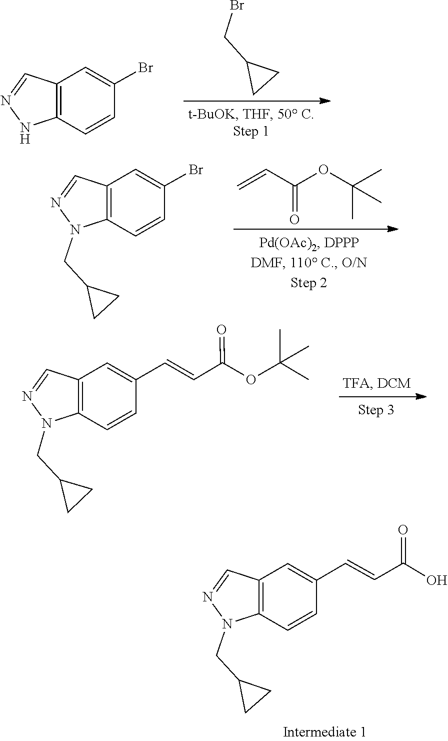

### Step 1:5-Bromo-1-(Cyclopropylmethyl)-1H-Indazole

To a solution of 5-bromo-1H-indazole (200.00 g, 1.02 mol) in THF (1200 mL) was added t-BuOK (136.68 g, 1.22 mol) and (Bromomethyl)cyclopropane (164.44 g, 1.22 mol) at 0° C. The reaction mixture was stirred at 50° C. overnight. The reaction mixture was treated with water and extracted with EtOAc (1000 mL*2), washed by brine (1000 mL) and dried over Na₂SO₄. The organic phase was concentrated in vacuum and purified by silica gel column chromatography (PE/EtOAc=30/1 to 10/1) to afford the title compound (125 g, 0.49 mol, 49.0% yield) as a yellow oil. ¹H NMR (400 MHz, DMSO-d₆) δ 8.01 (dd, J=20.2, 1.4 Hz, 2H), 7.74-7.66 (m, 1H), 7.47 (dd, J=8.9, 1.8 Hz, 1H), 4.29 (d, J=7.0 Hz, 2H), 1.32-1.12 (m, 1H), 0.51-0.40 (m, 2H), 0.43-0.30 (m, 2H).

Step 2: tert-butyl (E)-3-(1-(cyclopropylmethyl)-1H-indazol-5-yl)acrylate

To a solution of 5-bromo-1-(cyclopropylmethyl)-1H-indazole (125.00 g, 0.49 mol) in DMF (500 mL) was added Pd (OAc) 2 (11.17 g, 0.05 mol), DPPP (41.06 g, 0.10 mol), TEA (100.73 g, 1.00 mol) and tert-butyl acrylate (318.99 g, 2.49 mol) under N₂ atmosphere and stirred at 110° C. overnight. The mixture was concentrated in vacuum and extracted with EtOAc (1000 ml*3), washed by brine (1000 mL*2) and dried over Na₂SO₄. The organic phase was concentrated in vacuum and purified by silica gel column chromatography (PE/EtOAc=40/1 to DCM/EtOAc=100/1) to afford the title compound (110.0 g, 0.37 mol, 74.1% yield) as a white solid. ¹H NMR (400 MHz, DMSO-d₆) δ 8.10 (d, J=0.9 Hz, 1H), 8.04 (d, J=1.5 Hz, 1H), 7.78 (dd, J=8.9, 1.5 Hz, 1H), 7.76-7.63 (m, 2H), 6.50 (d, J=15.9 Hz, 1H), 4.31 (d, J=7.0 Hz, 2H), 1.49 (s, 9H), 1.44-1.20 (m, 1H), 0.53-0.42 (m, 2H), 0.46-0.35 (m, 2H).

Step 3: (E)-3-(1-(cyclopropylmethyl)-1H-indazol-5-yl)acrylic acid

To a solution of tert-butyl (E)-3-(1-(cyclopropylmethyl)-1H-indazol-5-yl)acrylate (110.0 g, 0.37 mol) in DCM (350 mL) was added TFA (840.7 g, 7.37 mol) at 0° C. The mixture reaction was stirred at RT for 2 hours. The mixture was concentrated under vacuum and washed by MeOH, and the filter cake was concentrated under vacuum to afford the title compound (76.0 g, 0.31 mol, 85.1% yield) as a white solid. ¹H NMR (400 MHz, DMSO-d₆) δ 12.28 (s, 1H), 8.13 (d, J=0.7 Hz, 1H), 8.06 (d, J=1.4 Hz, 1H), 7.83-7.69 (m, 3H), 6.53 (d, J=15.9 Hz, 1H), 4.34 (d, J=7.0 Hz, 2H), 1.35-1.23 (m, 1H), 0.55-0.44 (m, 2H), 0.48-0.37 (m, 2H).

### Intermediate 2. (E)-3-(1-methyl-1H-indazol-5-yl)acrylic acid

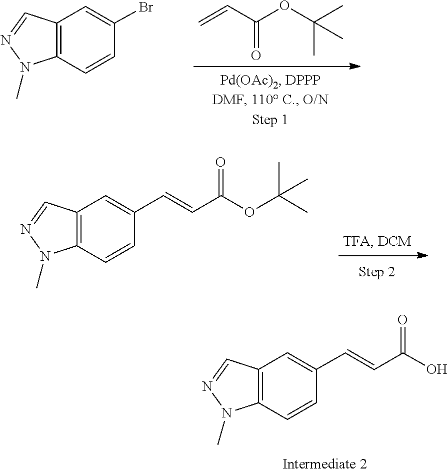

Step 1: tert-butyl (E)-3-(1-methyl-1H-indazol-5-yl)acrylate

Using the procedure outlined in Step 2 of Intermediate 1, starting with 5-bromo-1-methyl-1H-indazole (10.0 g, 47.62 mmol). The title compound was obtained (7.0 g, 27.13 mmol, 57% yield) as a white solid. ¹H NMR (400 MHz, DMSO-d₆) δ 8.08 (s, 1H), 8.03 (s, 1H), 7.80 (s, 1H), 7.66 (s, 2H), 6.47 (s, 1H), 4.02 (s, 3H), 1.49 (s, 9H).

Step 2: (E)-3-(1-methyl-1H-indazol-5-yl)acrylic acid

Using the procedure outlined in Step 3 of Intermediate 1, starting with tert-butyl (E)-3-(1-methyl-1H-indazol-5-yl)acrylate (7.0 g, 27.13 mmol). The title compound was obtained (5.0 g, 24.75 mmol, 91% yield) as a white solid. ¹H NMR (400 MHz, DMSO-d₆) δ 12.15 (s, 1H), 8.10 (s, 1H), 8.04 (s, 1H), 7.79 (d, J=8.9 Hz, 1H), 7.71 (d, J=16.0 Hz, 1H), 7.66 (d, J=8.8 Hz, 1H), 6.51 (d, J=15.9 Hz, 1H), 4.05 (s, 3H).

### Intermediate 3. 3-(2-aminophenyl)-1,2,4-oxadiazol-5 (4H)-one

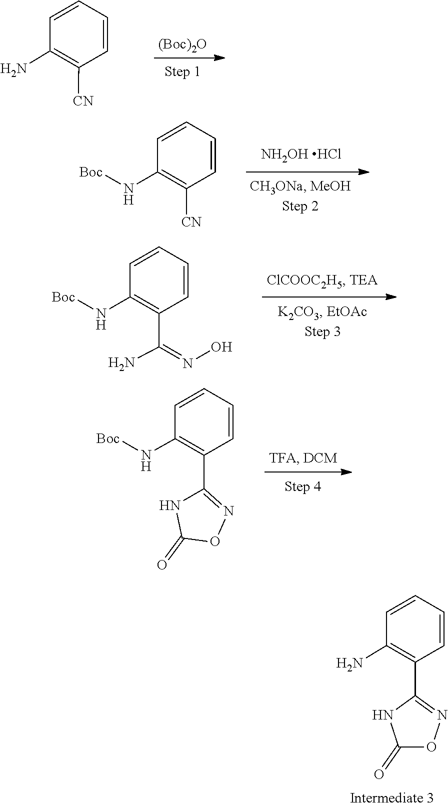

Step 1: tert-butyl(2-cyanophenyl)carbamate

To a solution of 2-aminobenzonitrile (1.0 g, 8.46 mmol) in DCM (30 mL) at RT was added di-tert-butyl dicarbonate (2.2 g, 10.15 mmol), TEA (1.28 g, 12.69 mmol), DMAP (100 mg, 0.85 mmol). The resulting solution was stirred at RT for 16 hours. The solvent was removed and treated with water, extracted with EtOAc (100 ml*3), washed by brine (100 mL*2) and dried over Na₂SO₄. The organic phase was concentrated in vacuum and purified by silica gel column chromatography (PE/EtOAc=10/1) to afford the title compound (700.0 mg, 3.2 mmol, 38% yield) as an colourless oil. ¹H NMR (400 MHz, Chloroform-d) δ 8.23 (d, J=8.5 Hz, 1H), 7.59-7.50 (m, 2H), 7.13-6.96 (m, 2H), 1.54 (d, J=1.5 Hz, 9H).

Step 2: tert-butyl (E)-(2-(N′-hydroxycarbamimidoyl)phenyl)carbamate

To a solution of tert-butyl(2-cyanophenyl)carbamate (700.0 mg, 3.2 mmol) in MeOH (10 mL) at RT was added hydroxylamine hydrochloride (449 mg, 6.4 mmol) and MeONa (346 mg, 6.4 mmol). The resulting solution was reflux for 16 hours. The solvent was removed and the crude product was purified by silica gel column chromatography (PE/EtOAc=1/1) to afford the title compound (350.0 mg, 1.4 mmol, 44% yield) as a brown solid. ¹H NMR (400 MHz, DMSO-d₆) δ 10.41 (s, 1H), 9.98 (s, 1H), 8.15 (dd, J=8.4, 1.2 Hz, 1H), 7.58 (dd, J=7.9, 1.6 Hz, 1H), 7.32 (ddd, J=8.6, 7.4, 1.6 Hz, 1H), 7.04 (td, J=7.6, 1.3 Hz, 1H), 6.12 (s, 2H), 1.47 (s, 9H).

Step 3: tert-butyl(2-(5-oxo-4,5-dihydro-1,2,4-oxadiazol-3-yl)phenyl)carbamate

To a solution of tert-butyl (E)-(2-(N′-hydroxycarbamimidoyl)phenyl)carbamate (350.0 mg, 1.4 mmol) in DCM (10 mL) at RT was added ethyl carbonochloridate (228 mg, 2.09 mmol) and TEA (282 mg, 2.78 mmol). The resulting solution was stirred at RT for 2 hours. The solvent was removed and treated with water, extracted with EtOAc (100 ml*3), washed by brine (100 mL*3) and dried over Na₂SO₄. The organic phase was concentrated in vacuum to give the crude product which was dissolved in EtOAc (10 mL) and K₂CO₃ (959 mg, 6.95 mmol) added. The mixture solution was refluxed for 16 hours. The solvent was removed and purified by silica gel column chromatography (DCM/MeOH=50/1) to afford the title compound (280.0 mg, 1.0 mmol, 71% yield) as a brown solid. ¹H NMR (400 MHz, DMSO-d₆) δ 12.84 (s, 1H), 9.21 (s, 1H), 7.94 (d, J=8.3 Hz, 1H), 7.61 (d, J=1.3 Hz, 2H), 7.29-7.17 (m, 1H), 1.46 (s, 9H).

Step 4:3-(2-aminophenyl)-1,2,4-oxadiazol-5 (4H)-one

The procedure outlined in Step 3 of Intermediate 1 was used, starting with tert-butyl(2-(5-oxo-4,5-dihydro-1,2,4-oxadiazol-3-yl)phenyl)carbamate (280.0 mg, 1.0 mmol). The title compound was obtained (177.0 mg, 1.0 mmol, 100% yield) as a white solid, then used in the next step directly without further purification. UPLC-MS (Method 3) m/z 178.0, (M+H)⁺ at 1.126 min.

### Intermediate 4. 3-(2-aminophenyl)-1,2,4-oxadiazol-5 (4H)-one

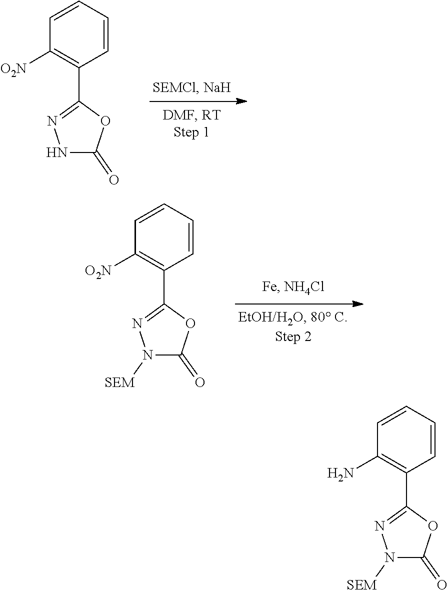

Step 1:5-(2-nitrophenyl)-3-((2-(trimethylsilyl)ethoxy)methyl)-1,3,4-oxadiazol-2 (3H)-one

To a solution of 5-(2-nitrophenyl)-1,3,4-oxadiazol-2 (3H)-one (600.0 mg, 2.9 mmol) in DMF (10 mL) at 0° C. was added NaH (66.6 mg, 2.9 mmol). The solution was stirred at the same temperature for 1 hour, then SEMCl (579.4 mg, 3.47 mmol) added. The resulting solution was stirred at RT for 16 hours. The solvent was treated with water, extracted with EtOAc (100 ml*3), washed by brine (100 mL*3) and dried over Na₂SO₄. The organic phase was concentrated in vacuum and purified by prep-TLC (PE/EtOAc=5/1) to afford the title compound (700.0 mg, 2.07 mmol, 71% yield) as a yellow solid. ¹H NMR (400 MHz, DMSO-d₆) δ 8.16 (s, 1H), 7.90 (s, 3H), 5.14 (s, 2H), 3.70-3.63 (m, 2H), 0.93-0.88 (m, 2H), -0.00 (s, 9H).

Step 2:5-(2-aminophenyl)-3-((2-(trimethylsilyl)ethoxy)methyl)-1,3,4-oxadiazol-2 (3H)-one

A mixture solution of 5-(2-nitrophenyl)-3-((2-(trimethylsilyl)ethoxy)methyl)-1,3,4-oxadiazol-2 (3H)-one (700 mg, 2.07 mmol), Fe powder (579.0 mg, 10.37 mmol) and NH₄Cl (555.0 mg, 10. 37 mmol) in EtOH/H₂O (16 mL, v/v=3:1) was stirred at 80° C. for 3 hours. The solvent was treated with water, extracted with EtOAc (100 ml*3), washed by brine (100 mL*2) and dried over Na₂SO₄. The organic phase was concentrated in vacuum to afford the title compound (380.0 mg, 1.23 mmol, 59% yield) as a white solid. ¹H NMR (400 MHz, DMSO-d₆) δ 7.48 (s, 1H), 7.25 (s, 1H), 6.87 (s, 1H), 6.67 (s, 1H), 6.38 (s, 2H), 5.12 (s, 2H), 3.70 (s, 2H), 0.93-0.88 (m, 2H), -0.02 (s, 9H).

### Example 3. Synthetic Route to Compound 6

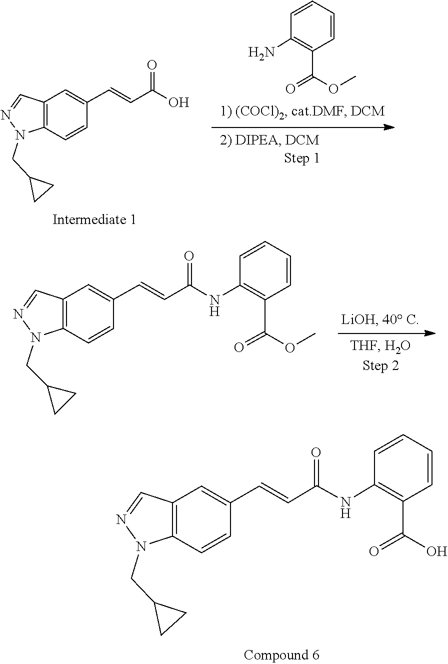 [SMILES: O=C(O)/C=C/c1ccc2c(cnn2CC2CC2)c1 ; COC(=O)c1ccccc1N ; COC(=O)c1ccccc1NC(=O)/C=C/c1ccc2c(cnn2CC2CC2)c1 ; O=C(/C=C/c1ccc2c(cnn2CC2CC2)c1)Nc1ccccc1C(=O)O]

Step 1: methyl (E)-2-(3-(1-(cyclopropylmethyl)-1H-indazol-5-yl)acrylamido)benzoate

To a mixture of (E)-3-(1-(cyclopropylmethyl)-1H-indazol-5-yl)acrylic acid (Intermediate 1, 76.0 g, 0.31 mol) in DCM (500 mL) were added (COCl)₂ (59.65 g, 0.47 mol) and cat. DMF at 0° C. The reaction was stirred at RT for 1 hour. The mixture reaction was concentrated and dissolved in DCM (500 mL), then methyl 2-aminobenzoate (47.42 g, 0.31 mol) and DIPEA (120.19 g, 0.93 mol) added. The resulting solution was stirred at RT for 3 hours. The mixture reaction was concentrated in vacuum and purified by silica gel column chromatography (PE/EtOAc=5/1 to DCM/EtOAc=30/1) to give a residue, which was washed by MeOH and filtered. The filter cake was concentrated in vacuum to afford the title compound (80 g, 67.9%) as a white solid. ¹H NMR (400 MHz, DMSO-d₆) δ 10.86 (s, 1H), 8.48 (dd, J=8.5, 1.1 Hz, 1H), 8.13 (d, J=16.2 Hz, 2H), 7.98 (dd, J=8.0, 1.6 Hz, 1H), 7.86 (dd, J=8.8, 1.5 Hz, 1H), 7.78 (d, J=15.9 Hz, 2H), 7.67 (ddd, J=8.6, 7.3, 1.7 Hz, 1H), 7.28-7.19 (m, 1H), 6.93 (d, J=15.6 Hz, 1H), 4.35 (d, J=6.9 Hz, 2H), 3.92 (s, 3H), 1.37-1.22 (m, 1H), 0.51 (dt, J=8.0, 2.8 Hz, 2H), 0.49-0.38 (m, 2H).

Step 2: (E)-2-(3-(1-(cyclopropylmethyl)-1H-indazol-5-yl)acrylamido)benzoic acid

A mixture of methyl (E)-2-(3-(1-(cyclopropylmethyl)-1H-indazol-5-yl)acrylamido)benzoate (100.0 g, 0.26 mol) and LiOH (33.48 g, 0.8 mmol) in THF (400 mL) and water (80 mL) was stirred at 40° C. for 2 hours. The solvent was removed under reduced pressure and the pH of the aqueous solution was adjusted to 3 with 2M HCl. The mixture was extracted with EtOAc, dried over Na₂SO₄ and concentrated under pressure to give the crude product. The crude product was washed with MeOH and the filter cake was dried under vacuum to afford the crude title compound (55.3 g, 0.15 mmol, 59% yield) as a white solid. UPLC-MS (Method 4) m/z 360.2, (M−H)⁻ at 2.185 min. ¹H NMR (400 MHz, DMSO-d₆) δ 13.60 (s, 1H), 11.33 (s, 1H), 8.64 (d, J=8.4 Hz, 1H), 8.11 (d, J=12.9 Hz, 2H), 8.02 (d, J=7.8 Hz, 1H), 7.84 (d, J=8.9 Hz, 1H), 7.82-7.71 (m, 2H), 7.63 (t, J=7.8 Hz, 1H), 7.17 (t, J=7.6 Hz, 1H), 6.87 (d, J=15.6 Hz, 1H), 4.32 (d, J=6.9 Hz, 2H), 1.29 (t, J=9.8 Hz, 1H), 0.49 (d, J=7.6 Hz, 2H), 0.41 (t, J=4.4 Hz, 2H).

### Example 4. Synthetic Route to Compound 9

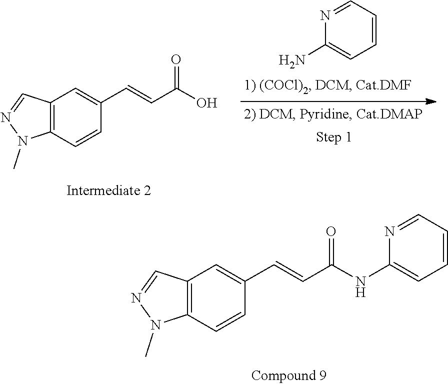 [SMILES: Cn1ncc2cc(/C=C/C(=O)O)ccc21 ; Nc1ccccn1 ; Cn1ncc2cc(/C=C/C(=O)Nc3ccccn3)ccc21]

Step 1: (E)-3-(1-methyl-1H-indazol-5-yl)-N-(pyridin-2-yl)acrylamide

To a mixture of (E)-3-(1-methyl-1H-indazol-5-yl)acrylic acid (Intermediate 2, 100.0 mg, 0.49 mol) in DCM (5 mL) was added (COCl)₂ (62.0 g, 0.49 mol) and cat.DMF at 0° C. The reaction was stirred at RT 1 hour. The mixture reaction was concentrated and dissolved in pyridine (1 mL), then added a solution of methyl 2-aminobenzoate (184.0 mg, 1.96 mol) in DCM (5 mL) and DMAP (6 mg, 0.05 mmol). The resulting solution was stirred at RT for 12 hours. The mixture reaction was concentrated in vacuum and purified by prep-TLC to give the title compound (47.0 mg, 0.17 mmol, 35%) as a white solid. UPLC-MS (Method 2) m/z 279.15 (M+H)⁺ at 3.017 min. ¹H NMR (400 MHz, DMSO-d₆) δ 10.68 (s, 1H), 8.37-8.33 (m, 1H), 8.23 (d, J=8.4 Hz, 1H), 8.13 (s, 1H), 8.01 (s, 1H), 7.83 (t, J=8.0 Hz, 1H), 7.79-7.66 (m, 3H), 7.13 (t, J=6.2 Hz, 1H), 7.03 (d, J=15.7 Hz, 1H), 4.07 (s, 3H).

### Example 5. Synthetic Route to Compound 12

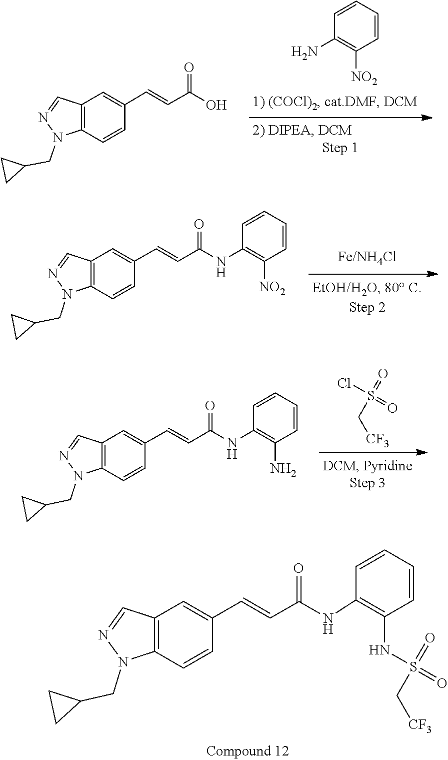 [SMILES: O=C(O)/C=C/c1ccc2c(cnn2CC2CC2)c1 ; Nc1ccccc1[N+](=O)[O-] ; O=C(/C=C/c1ccc2c(cnn2CC2CC2)c1)Nc1ccccc1[N+](=O)[O-] ; Nc1ccccc1NC(=O)/C=C/c1ccc2c(cnn2CC2CC2)c1 ; O=S(=O)(Cl)CC(F)(F)F ; O=C(/C=C/c1ccc2c(cnn2CC2CC2)c1)Nc1ccccc1NS(=O)(=O)CC(F)(F)F]

Step 1: (E)-3-(1-(cyclopropylmethyl)-1H-indazol-5-yl)-N-(2-nitrophenyl)acrylamide

Using the procedure outlined in Step 1 of Example 1, starting with (E)-3-(1-(cyclopropylmethyl)-1H-indazol-5-yl)acrylic acid (500.0 mg, 2.06 mmol). The title compound was obtained (500.0 mg, 1.38 mmol, 67% yield) as a yellow solid. ¹H NMR (400 MHz, DMSO-d₆) δ 10.47 (s, 1H), 8.14 (d, J=0.9 Hz, 1H), 8.05 (s, 1H), 7.98 (s, 1H), 7.85-7.78 (m, 2H), 7.77-7.71 (m, 3H), 7.40 (s, 1H), 6.88 (s, 1H), 4.32 (s, 2H), 1.29-1.25 (m, 1H), 0.53-0.47 (m, 2H), 0.40 (s, 2H).

Step 2: (E)-N-(2-aminophenyl)-3-(1-(cyclopropylmethyl)-1H-indazol-5-yl)acrylamide

Using the procedure outlined in Step 2 of Intermediate 4, starting with (E)-3-(1-(cyclopropylmethyl)-1H-indazol-5-yl)-N-(2-nitrophenyl)acrylamide (500.0 mg, 1.38 mmol). The title compound was obtained (400.0 mg, 1.2 mmol, 87% yield) as a yellow solid. ¹H NMR (400 MHz, DMSO-d₆) δ 9.34 (s, 1H), 8.13 (s, 1H), 7.99 (s, 1H), 7.78 (d, J=8.8 Hz, 1H), 7.72-7.62 (m, 2H), 7.35 (d, J=7.9 Hz, 1H), 6.96-6.83 (m, 2H), 6.76 (dd, J=8.0, 1.4 Hz, 1H), 6.58 (td, J=7.6, 1.4 Hz, 1H), 4.95 (s, 2H), 4.32 (d, J=7.0 Hz, 2H), 1.28 (d, J=2.9 Hz, 1H), 0.49 (dt, J=7.8, 2.9 Hz, 2H), 0.43-0.37 (m, 2H).

Step 3: (E)-3-(1-(cyclopropylmethyl)-1H-indazol-5-yl)-N-(2-((2,2,2-trifluoroethyl)sulfonamido)phenyl)acrylamide

To a mixture of (E)-N-(2-aminophenyl)-3-(1-(cyclopropylmethyl)-1H-indazol-5-yl)acrylamide (150.0 mg, 0.45 mol) in DCM (500 mL) at 0° C. was added pyridine (71.0 mg, 0.90 mol) and 2,2,2-trifluoroethane-1-sulfonyl chloride (91.0 mg, 0.50). The reaction was stirred at RT for 1 hour. The solvent was treated with water, extracted with EtOAc (50 ml*3), washed by brine (50 mL*2) and dried over Na₂SO₄. The organic phase was concentrated in vacuum to give the crude product and purified by prep-HPLC (eluting with 10% to 90% MeCN/H₂O, contained 0.1% HCOOH) to afford the title compound (80.0 mg, 0.17 mmol, 38% yield) as a yellow solid. UPLC-MS (Method 1) m/z 477.1, (M−H)⁻ at 2.300 min. ¹H NMR (400 MHz, DMSO-d₆) δ 9.73 (s, 1H), 9.66 (s, 1H), 8.14 (s, 1H), 8.05 (s, 1H), 7.82-7.70 (m, 4H), 7.51-7.44 (m, 1H), 7.35-7.19 (m, 2H), 6.94 (d, J=15.6 Hz, 1H), 4.55 (q, J=9.8 Hz, 2H), 4.33 (d, J=6.9 Hz, 2H), 1.29 (ddt, J=12.5, 7.7, 3.9 Hz, 1H), 0.53-0.45 (m, 2H), 0.41 (h, J=5.5, 5.0 Hz, 2H).

### Example 6. Analytical Data for Example Synthesised Compounds

The following example compounds were prepared by methods described in Examples 3 to 5 hereinabove; or by methods analogous to the methods described hereinabove, substituting appropriate starting materials and intermediates where necessary.

| No. | Compound Structure | Name/Analytical Data |
|---|---|---|
| 4 | 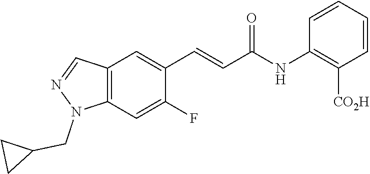 [SMILES: O=C(/C=C/c1cc2cnn(CC3CC3)c2cc1F)Nc1ccccc1C(=O)O] | (E)-2-(3-(1-(cyclopropylmethyl)-6-fluoro-1H- indazol-5-yl)acrylamido)benzoic acid. UPLC-MS (Method 2) m/z 380.10, (M + H)⁺ at 4.350 min. ¹H NMR (400 MHz, DMSO-d₆) δ 13.95- 13.22 (m, 1H), 11.40 (s, 1H), 8.61 (d, J = 8.3 Hz, 1H), 8.35 (d, J = 7.2 Hz, 1H), 8.16 (s, 1H), 8.01 (dd, J = 8.0, 1.6 Hz, 1H), 7.76 (dd, J = 16.7, 13.8 Hz, 2H), 7.63 (td, J = 8.4, 7.8, 1.7 Hz, 1H), 7.22- 7.14 (m, 1H), 6.92 (d, J = 15.7 Hz, 1H), 4.28 (d, J = 7.0 Hz, 2H), 1.27 (d, J = 7.2 Hz, 1H), 0.49 (dt, J = 7.8, 2.8 Hz, 2H), 0.41 (dt, J = 5.1, 2.7 Hz, 2H). |
| 6 | 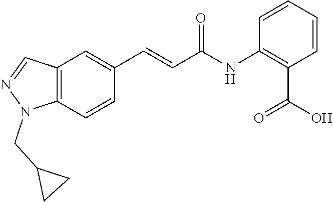 [SMILES: O=C(/C=C/c1ccc2c(cnn2CC2CC2)c1)Nc1ccccc1C(=O)O] | (E)-2-(3-(1-(cyclopropylmethyl)-1H-indazol-5- yl)acrylamido)benzoic acid. UPLC-MS (Method 4) m/z 360.2, (M − H)⁻; at 2.185 min. ¹H NMR (400 MHz, DMSO-d₆) δ 13.60 (s, 1H), 11.33 (s, 1H), 8.64 (d, J = 8.4 Hz, 1H), 8.11 (d, J = 12.9 Hz, 2H), 8.02 (d, J = 7.8 Hz, 1H), 7.84 (d, J = 8.9 Hz, 1H), 7.82- 7.71 (m, 2H), 7.63 (t, J = 7.8 Hz, 1H), 7.17 (t, J = 7.6 Hz, 1H), 6.87 (d, J = 15.6 Hz, 1H), 4.32 (d, J = 6.9 Hz, 2H), 1.29 (t, J = 9.8 Hz, 1H), 0.49 (d, J = 7.6 Hz, 2H), 0.41 (t, J = 4.4 Hz, 2H). |
| 9 | 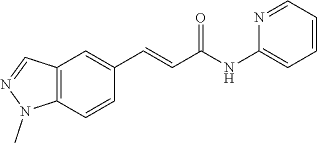 [SMILES: Cn1ncc2cc(/C=C/C(=O)Nc3ccccn3)ccc21] | (E)-3-(1-methyl-1H-indazol-5-yl)-N-(pyridin-2- yl)acrylamide. UPLC-MS (Method 2) m/z 279.15 (M + H)⁺ at 3.017 min. ¹H NMR (400 MHz, DMSO-d₆) δ 10.68 (s, 1H), 8.37- 8.33 (m, 1H), 8.23 (d, J = 8.4 Hz, 1H), 8.13 (s, 1H), 8.01 (s, 1H), 7.83 (t, J = 8.0 Hz, 1H), 7.79- 7.66 (m, 3H), 7.13 (t, J = 6.2 Hz, 1H), 7.03 (d, J = 15.7 Hz, 1H), 4.07 (s, 3H). |
| 5 | 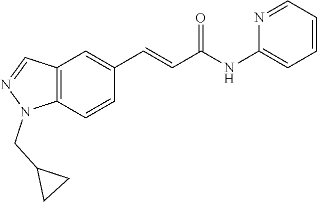 [SMILES: O=C(/C=C/c1ccc2c(cnn2CC2CC2)c1)Nc1ccccn1] | (E)-3-(1-(cyclopropylmethyl)-1H-indazol-5-yl)-N- (pyridin-2-yl)acrylamide. UPLC-MS (Method 1) m/z 319.10 (M + H)⁺ at 1.700 min. ¹H NMR (400 MHz, DMSO-d₆) δ 10.76- 10.62 (m, 1H), 8.38- 8.32 (m, 1H), 8.26- 8.19 (m, 1H), 8.15 (s, 1H), 8.01 (s, 1H), 7.85 (d, J = 9.2 Hz, 1H), 7.81- 7.74 (m, 2H), 7.66 (d, J = 9.1 Hz, 1H), 7.13 (s, 1H), 7.02 (d, J = 15.8 Hz, 1H), 4.32 (d, J = 7.0 Hz, 2H), 1.32- 1.26 (m, 1H), 0.52- 0.47 (m, 2H), 0.40 (d, J = 5.0 Hz, 2H). |
| 7 | 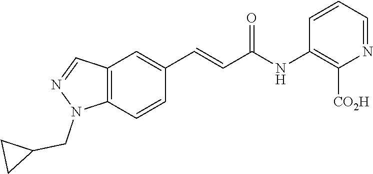 [SMILES: O=C(/C=C/c1ccc2c(cnn2CC2CC2)c1)Nc1cccnc1C(=O)O] | (E)-3-(3-(1-(cyclopropylmethyl)-1H-indazol-5- yl)acrylamido)picolinic acid. UPLC-MS (Method 4) m/z 363.10, (M + H)⁺ at 1.442 min. ¹H NMR (400 MHz, DMSO-d₆) δ 11.51 (s, 1H), 9.04 (d, J = 8.3 Hz, 1H), 8.46- 8.33 (m, 1H), 8.13 (s, 1H), 8.11 (s, 1H), 7.85 (d, J = 8.9 Hz, 1H), 7.80 (s, 1H), 7.76 (s, 1H), 7.73 (d, J = 8.6 Hz, 1H), 6.93 (d, J = 15.6 Hz, 1H), 4.33 (d, J = 7.0 Hz, 2H), 1.35- 1.25 (m, 1H), 0.50 (s, 2H), 0.40 (dd, J = 5.0, 1.7 Hz, 2H). |
| 2 | 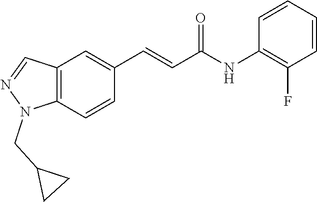 [SMILES: O=C(/C=C/c1ccc2c(cnn2CC2CC2)c1)Nc1ccccc1F] | (E)-3-(1-(cyclopropylmethyl)-1H-indazol-5-yl)-N- (2-fluorophenyl)acrylamide. UPLC-MS (Method 1) m/z 334.0, (M − H)⁻; at 1.916 min. ¹H NMR (400 MHz, DMSO-d₆) δ 9.86 (s, 1H), 8.13 (d, J = 4.3 Hz, 2H), 8.00 (s, 1H), 7.79 (d, J = 8.8 Hz, 1H), 7.72 (d, J = 15.7 Hz, 1H), 7.67 (d, J = 8.9 Hz, 1H), 7.28 (dd, J = 11.1, 9.6 Hz, 1H), 7.22- 7.13 (m, 2H), 7.06 (d, J = 15.7 Hz, 1H), 4.32 (d, J = 7.0 Hz, 2H), 1.34- 1.24 (m, 1H), 0.52- 0.47 (m, 2H), 0.40 (q, J = 6.2, 5.6 Hz, 2H). |
| 3 | 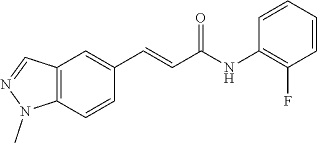 [SMILES: Cn1ncc2cc(/C=C/C(=O)Nc3ccccc3F)ccc21] | (E)-N-(2-fluorophenyl)-3-(1-methyl-1H-indazol-5- yl)acrylamide. UPLC-MS (Method 4) m/z 296.0, (M + H)⁺ at 1.880 min. ¹H NMR (400 MHz, DMSO-d₆) δ 10.69 (s, 1H), 8.58- 8.44 (m, 2H), 7.57- 7.53 (m, 2H), 7.50 (td, J = 8.7, 3.1 Hz, 1H), 7.24 (dd, J = 9.2, 4.1 Hz, 1H), 4.99 (s, 2H). |
| 10 | 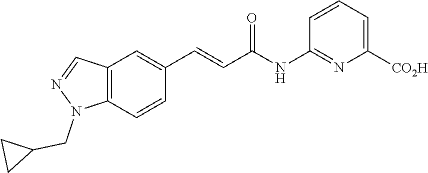 [SMILES: O=C(/C=C/c1ccc2c(cnn2CC2CC2)c1)Nc1cccc(C(=O)O)n1] | (E)-6-(3-(1-(cyclopropylmethyl)-1H-indazol-5- yl)acrylamido)picolinic acid. UPLC-MS (Method 2) m/z 363.20, (M + H)⁺ at 3.517 min. ¹H NMR (400 MHz, DMSO-d₆) δ 10.92 (s, 1H), 8.45 (d, J = 8.3 Hz, 1H), 8.14 (s, 1H), 8.01- 7.96 (m, 2H), 7.80- 7.75 (m, 3H), 7.65 (d, J = 8.8 Hz, 1H), 7.08 (d, J = 15.6 Hz, 1H), 4.32 (d, J = 7.0 Hz, 2H), 1.29 (d, J = 12.0 Hz, 1H), 0.49 (d, J = 7.6 Hz, 2H), 0.40 (d, J = 5.0 Hz, 2H). |
| 11 | 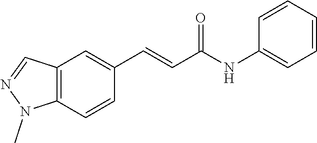 [SMILES: Cn1ncc2cc(/C=C/C(=O)Nc3ccccc3)ccc21] | (E)-3-(1-methyl-1H-indazol-5-yl)-N- phenylacrylamide. UPLC-MS (Method 2) m/z 278.20, (M + H)⁺ at 3.683 min. ¹H NMR (400 MHz, DMSO-d₆) δ 10.15 (s, 1H), 8.11 (s, 1H), 8.00 (s, 1H), 7.72 (d, J = 4.7 Hz, 1H), 7.70 (d, J = 3.9 Hz, 4H), 7.33 (t, J = 7.8 Hz, 2H), 7.06 (t, J = 7.4 Hz, 1H), 6.82 (d, J = 15.7 Hz, 1H), 4.06 (s, 3H). |
| 8 | 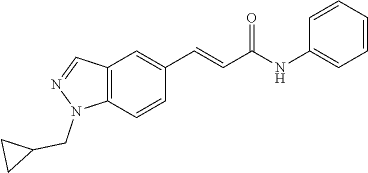 [SMILES: O=C(/C=C/c1ccc2c(cnn2CC2CC2)c1)Nc1ccccc1] | (E)-3-(1-(cyclopropylmethyl)-1H-indazol-5-yl)-N- phenylacrylamide. UPLC-MS (Method 2) m/z 318.20, (M + H)⁺ at 4.150 min. ¹H NMR (400 MHz, DMSO-d₆) δ 10.13 (s, 1H), 8.13 (s, 1H), 8.00 (s, 1H), 7.79 (d, J = 8.8 Hz, 1H), 7.71 (dd, J = 11.8, 3.8 Hz, 4H), 7.33 (t, J = 7.8 Hz, 2H), 7.06 (t, J = 7.3 Hz, 1H), 6.82 (d, J = 15.6 Hz, 1H), 4.32 (d, J = 6.9 Hz, 2H), 1.32- 1.24 (m, 1H), 0.49 (q, J = 5.2, 3.9 Hz, 2H), 0.40 (q, J = 5.9, 5.2 Hz, 2H). |
| 18 | 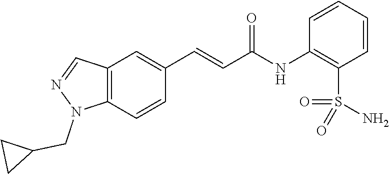 [SMILES: NS(=O)(=O)c1ccccc1NC(=O)/C=C/c1ccc2c(cnn2CC2CC2)c1] | (E)-3-(1-(cyclopropylmethyl)-1H-indazol-5-yl)-N- (2-sulfamoylphenyl)acrylamide. UPLC-MS (Method 4) m/z 395.10, (M − H)⁻; at 2.009 min. ¹H NMR (400 MHz, DMSO-d₆) δ 9.55 (s, 1H), 8.38- 8.34 (m, 1H), 8.14 (s, 1H), 8.09 (s, 1H), 7.88 (d, J = 9.4 Hz, 1H), 7.78 (d, J = 10.7 Hz, 3H), 7.73 (s, 2H), 7.62 (t, J = 8.5 Hz, 1H), 7.30 (t, J = 8.2 Hz, 1H), 6.87 (d, J = 15.6 Hz, 1H), 4.33 (d, J = 6.9 Hz, 2H), 1.29 (t, J = 7.7 Hz, 1H), 0.53- 0.47 (m, 2H), 0.41 (q, J = 4.9 Hz, 2H). |
| 16 | 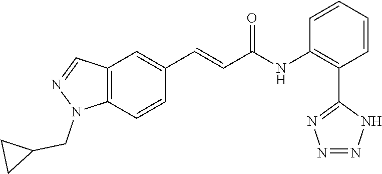 [SMILES: O=C(/C=C/c1ccc2c(cnn2CC2CC2)c1)Nc1ccccc1-c1nnn[nH]1] | (E)-N-(2-(1H-tetrazol-5-yl)phenyl)-3-(1- (cyclopropylmethyl)-1H-indazol-5-yl)acrylamide. UPLC-MS (Method 4) m/z 384.1, (M − H)⁻; at 2.071 min. ¹H NMR (400 MHz, DMSO-d₆) δ 10.80 (s, 1H), 8.45 (dd, J = 8.5, 1.1 Hz, 1H), 8.14 (s, 1H), 8.08 (s, 1H), 7.94 (dd, J = 7.9, 1.5 Hz, 1H), 7.76 (d, J = 15.8 Hz, 3H), 7.60 (td, J = 8.3, 7.9, 1.6 Hz, 1H), 7.34 (td, J = 7.6, 1.2 Hz, 1H), 6.86 (d, J = 15.6 Hz, 1H), 4.33 (d, J = 6.9 Hz, 2H), 1.28 (ddd, J = 12.5, 5.8, 3.7 Hz, 1H), 0.53- 0.47 (m, 2H), 0.44- 0.37 (m, 2H). |
| 14 | 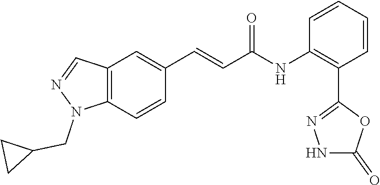 [SMILES: O=C(/C=C/c1ccc2c(cnn2CC2CC2)c1)Nc1ccccc1-c1n[nH]c(=O)o1] | (E)-3-(1-(cyclopropylmethyl)-1H-indazol-5-yl)-N- (2-(5-oxo-4,5-dihydro-1,3,4-oxadiazol-2- yl)phenyl)acrylamide. UPLC-MS (Method 1) m/z 400.10, (M − H)⁻ at 1.750 min. ¹H NMR (400 MHz, DMSO-d₆) δ 12.75 (s, 1H), 10.11 (s, 1H), 8.23 (d, J = 8.1 Hz, 1H), 8.14 (s, 1H), 8.05 (s, 1H), 7.81- 7.72 (m, 4H), 7.58 (t, J = 7.8 Hz, 1H), 7.30 (t, J = 7.7 Hz, 1H), 6.82 (d, J = 15.7 Hz, 1H), 4.33 (d, J = 6.9 Hz, 2H), 1.29 (t, J = 7.2 Hz, 1H), 0.49 (d, J = 8.0 Hz, 2H), 0.41 (d, J = 3.8 Hz, 2H). |
| 12 | 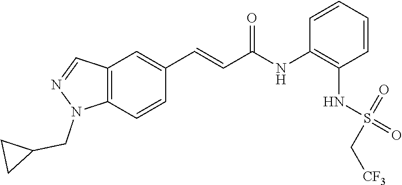 [SMILES: O=C(/C=C/c1ccc2c(cnn2CC2CC2)c1)Nc1ccccc1NS(=O)(=O)CC(F)(F)F] | (E)-3-(1-(cyclopropylmethyl)-1H-indazol-5-yl)-N- (2-((2,2,2- trifluoroethyl)sulfonamido)phenyl)acrylamide. UPLC-MS (Method 1) m/z 477.1, (M − H)⁻; at 2.300 min. ¹H NMR (400 MHz, DMSO-d₆) δ 9.73 (s, 1H), 9.66 (s, 1H), 8.14 (s, 1H), 8.05 (s, 1H), 7.82- 7.70 (m, 4H), 7.51- 7.44 (m, 1H), 7.35- 7.19 (m, 2H), 6.94 (d, J = 15.6 Hz, 1H), 4.55 (q, J = 9.8 Hz, 2H), 4.33 (d, J = 6.9 Hz, 2H), 1.29 (ddt, J = 12.5, 7.7, 3.9 Hz, 1H), 0.53- 0.45 (m, 2H), 0.41 (h, J = 5.5, 5.0 Hz, 2H). |
| 13 | 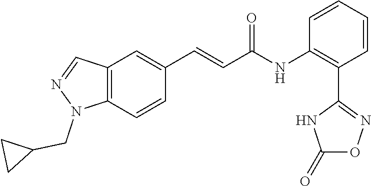 [SMILES: O=C(/C=C/c1ccc2c(cnn2CC2CC2)c1)Nc1ccccc1-c1noc(=O)[nH]1] | (E)-3-(1-(cyclopropylmethyl)-1H-indazol-5-yl)-N- (2-(5-oxo-4,5-dihydro-1,2,4-oxadiazol-3- yl)phenyl)acrylamide. UPLC-MS (Method 1) m/z 402.10, (M + H)⁺ at 1.600 min. ¹H NMR (400 MHz, DMSO-d₆) δ 12.80 (s, 1H), 10.02 (s, 1H), 8.15 (d, J = 7.4 Hz, 2H), 8.04 (s, 1H), 7.82- 7.70 (m, 3H), 7.68- 7.58 (m, 2H), 7.32 (t, J = 7.6 Hz, 1H), 6.85 (d, J = 15.6 Hz, 1H), 4.32 (d, J = 7.0 Hz, 2H), 1.28 (tt, J = 8.0, 3.9 Hz, 1H), 0.49 (dd, J = 7.7, 4.6 Hz, 2H), 0.41 (t, J = 4.8 Hz, 2H). |

### Example 7. Biological Testing

### GPR68 CAMP GloSensor Assay Protocol

The assay was developed to provide a quick screen assay to determine the potency and EC50s/IC50s of compounds against the GPR68 receptor with transient transfection cell.

### Assay Design Principle

GPR68 senses extracellular pH. Levels of cyclic adenosine monophosphate (cAMP), a secondary messenger associated with activation of GPCRs in the cAMP-dependent pathway, were found to be elevated in neutral to acidic extracellular pH (pH 7.0-6.5) in cells expressing GPR68. The pH-sensing ability of GPR68 was further tested and confirmed, as it was found that CAMP levels increased when GPR68 was stimulated by pH values less than pH 7.2.

To determine the compound activity in GPR68 inhibition, an GPR68 dependent cAMP assay was developed and validated. Principle of this method: Expression of GPR68 in HEK293T cells by transient transfection of a GPR68-expression vector and a cAMP-sensing reporter vector (GloSensor, Promega). The transfected cells as a pool will be re-plated uniformly in assay plates, to minimize the in-experiment variation. The GPR68 receptor signal will be measured via Gs to CAMP increase as indicated by the Luc activation under basal (pH8.4 or pH7.4) or activated (pH6.5 or pH6.0) states.

### Reagent List

GloSensor™ CAMP Reagent (Promega, Cat #E1291)
human GPR68 NM_001177676.2_pcDNA3.1 (+)(Genscript)
The GloSensor™-22F CAMP Reporter Vector (Promega, Cat #E2301)
DMEM (Gibco, Cat #11995065)
FBS, Qualified, New Zealand origin (Gibco, Cat #10091148)
Penicillin-Streptomycin (Gibco, Cat #15140122)
0.25% Trypsin-EDTA (Gibco, Cat #25200056)
PBS (Gibco, Cat #10010049)
1x HBSS (Gibco, Cat #14175079)
HEPES (Gibco, Cat #15630080)
MES (Sigma, Cat #M2933-25G)
TAPS (Sigma, Cat #T5316-25G)
NaOH (Sigma, Cat #221465)
BSA (Sigma, Cat #B2064-50G)
Opti-MEM (Gibco, Cat #31985070)
DMSO (Sigma, Cat #D5879-1L)
FuGene HD Transfection Reagent (Promega, Cat #E2311)

### Consumables/Supplies List

96 well plate (Corning, Cat #3610)
6-well plate (Corning, Cat #3516)
96 well V bottom plate (Greiner Bio-one, Cat #651201)
Cell culture dish 100 mm (Corning, Cat #CLS430167)

### Equipment & Settings

Liquid handler: Echo (LABCYTE Echo550), BRAVO (Agilent)
Incubator (Grant-bio Thermo-shaker PHMP)
Plate Centrifuge (Eppendorf 5910R)
Plate reader: Envision (PE).

### Experimental Procedures

### Cell Culture Procedures

### 1. Thawing Cells

1) Quickly thawed frozen cells at 37° C. water bath with a gently continuous agitation.
2) Gently added the cells, drop by drop, to a 15 ml centrifuge tube containing 10 ml of fresh pre-warmed complete medium. Then centrifuged the cells at 1000 rpm for 5 minutes.
3) Discarded the supernatant medium and resuspended the cell pellet in 10 ml of fresh pre-warmed complete medium. Transferred cells to a 10 cm dish and incubated at 37° C. with 5% CO₂ until the cells reach >90% confluence. The recovery rate for frozen cells was usually 80% or above.

### 2. Subculturing

This cell line was normally split twice every 1-2 days at a 1:3 to 1:5 ratio dilutions. The fresh cells typically needed 1-2 days to grow to 85% confluence.
1) Carefully aspirated all the media, washed the cell monolayer with 5 mL pre-warmed PBS for 10 cm dish gently, rinsed the cell layer with appropriate amount (gently 1 mL for 10 cm dish) of 0.2% trypsin-EDTA, and then aspirated it off.
2) Added 5-fold of trypsin volume culture medium to stop the trypsin, harvested cells by centrifuged at 1000 rpm for 5 min at RT.
3)Resuspended cells with appropriate amount of complete medium, and split cells as desired.

### 3. Changing Medium

1) Gently aspirated off the medium.
2) Transferred fresh warm complete medium (37° C.) into the 10 cm dish.

### 4. Freezing Cells

1)Repeated subculturing steps 1-3.
2) Centrifuged down the cells at 1000 rpm for 5 min.
3) Aspirated off the supernatant and resuspended the cells in fresh freezing medium at a density of 1-2×10⁶ cells/ml. Added 1 ml cells per Cryogenic Vial.
4) Put the Cryogenic Vial of cells into Cryo Freezing Container followed by transferring the container into −80° C. and staying overnight.
5) Transferred Cryogenic Vial into liquid nitrogen (−196° C.).

### Assay Procedures

### Step 1: Seeded Cells in 6-Well Plate

1) Detached cells with 0.25% Trypsin when the cell confluence was about 80%.
2)Resuspended the detached cells in 3 ml fresh cell culture medium. Counted the cell number and diluted the cells to proper density.
3) Seeded 1.5∧10∧6 cells into 6-well plate (Cat. #3516) in 2 ml per well contained the need to transfection well so that the cells were 80-85% confluent at the time of transfection.
4) Incubated the cells at 37° C. in a CO₂ incubator for 6 hours.

### Step 2: Transfected in 6-Well Plate

5) Gently aspirated off the DMEM medium. Then added the Opti-MEM® Medium without serum into 6-well plate.
6) Added 2.5 μg GPR68 and 2.5 μg GloSensor-22F in 1:1 ratio and 12 μl FuGeneHD into 120 μl of Opti-MEM® Medium without serum. Mixed gently and incubated for 15 min at room temperature.
7) After the 15-minute incubation, added 137 μl of DNA-Transfection Reagent complexes to each well containing cells and medium. Mixed gently by rocking the plate back and forth.
8) Incubated the cells at 37° C. in a co₂ incubator for 19 h until they were ready to assay for transgene expression.

### Step 3: Re-Seeded Cells in 96-Well Plate

Seeded 40000 cells/well, 100 μl/well in 96-well plate (Cat. #3610) with complete DMEM medium; incubated overnight.

### Step 4: Test Compounds

1. Prepared different pH buffers.

| | Stock | Final | | Buffer 1 | Buffer 2 | Buffer 3 | Buffer 4 |
|---|---|---|---|---|---|---|---|
| Item | (mM) | (mM) | Fold | (pH 6.0) | (pH 5.82) | (pH 7.4) | (pH 8.4) |
| 1 × HBSS | 1X | 1X | 1 | 47000 | 47000 | 47000 | 47000 |
| MES | 1000 | 20 | 50 | 1000 | 1000 | — | — |
| TAPS | 1000 | 20 | 50 | — | — | — | 1000 |
| HEPES | 1000 | 20 | 50 | — | — | 1000 | — |
| BSA | 10% | 0.1% | 100 | 500 | 500 | 500 | 500 |
| NaOH (based | — | — | — | | | | |
| on pH | | | | | | | |
| detection) | | | | | | | |
| NaCl | 5000 | 130 | 38.46 | 1300 | 1300 | 1300 | 1100 |
| 1 × HBSS | — | — | — | | | | |
| supplemented | | | | | | | |
| Total(ul) | — | — | — | 50000 | 50000 | 50000 | 50000 |

### 2. Prepared Compounds

1) Compounds were solubilized in 100% DMSO to a concentration of 50 mM.
2) Prepared compound DMSO solutions in 384 LDV echo plate: top concentration was 10 mM, 3-fold, 10 dose-response, so intermediate concentrations were created by serially diluting by (1:2) beginning from the 10000 μM. 5 μL tested intermediate compounds+10 μl DMSO solutions were transferred to 384 LDV echo plate. Centrifuged the echo plate at 1000 rpm for 30s before Echo procedure. Compounds were transferred 180 nL volume by Echo550 to a 96-assay plate (corning, Cat #3599), to make compound 10 concentration points in duplicate (the final top concentration was 10 μM, 3-fold, 10 dose-response, in duplicate). Final DMSO concentration is 0.1%.
3) For the Low control wells and High control wells, 180 nL DMSO were transferred by ECHO.
4) Layout of assay plate 180 nL/well of compounds in 100% DMSO 180 nL/well DMSO in Low control wells and High control wells was as shown below. Compounds were prepared into assay plate at 100×working concentration in 100% DMSO, the assay volume was 150 μL, and final DMSO concentration was 0.1%.

| | | 1 | 2 | 3 | 4 | 5 | 6 | 7 | 8 | 9 | 10 | 11 | 12 |
|---|---|---|---|---|---|---|---|---|---|---|---|---|---|
| Cpd | A | DMSO | 10000 | 3333 | 1111 | 370.37 | 123.46 | 41.15 | 13.72 | 4.572 | 1.524 | 0.5081 | DMSO |
| 1 | B | (low | 10000 | 3333 | 1111 | 370.37 | 123.46 | 41.15 | 13.72 | 4.572 | 1.524 | 0.5081 | (high |
| Cpd | C | control) | 10000 | 3333 | 1111 | 370.37 | 123.46 | 41.15 | 13.72 | 4.572 | 1.524 | 0.5081 | control) |
| 2 | D | | 10000 | 3333 | 1111 | 370.37 | 123.46 | 41.15 | 13.72 | 4.572 | 1.524 | 0.5081 | |
| Cpd | E | | 10000 | 3333 | 1111 | 370.37 | 123.46 | 41.15 | 13.72 | 4.572 | 1.524 | 0.5081 | |
| 3 | F | | 10000 | 3333 | 1111 | 370.37 | 123.46 | 41.15 | 13.72 | 4.572 | 1.524 | 0.5081 | |
| Cpd | G | | 10000 | 3333 | 1111 | 370.37 | 123.46 | 41.15 | 13.72 | 4.572 | 1.524 | 0.5081 | |
| 4 | H | | 10000 | 3333 | 1111 | 370.37 | 123.46 | 41.15 | 13.72 | 4.572 | 1.524 | 0.5081 | |

10000 nM top, 3-fold dilution, 10 points, in duplicates

### 3. Luciferin Loading

1)Removed culture medium, added 100 μL/well GloSensor (50×) to cells, then incubated for 2 h at room temperature.

| | Stock (mg/mL) | Final (mg/mL) | Fold | Add (μL) |
|---|---|---|---|---|
| GloSensor | 30 | 0.6 | 50 | 220 |
| Buffer 4 (pH 8.4) | | | | 10780 |
| Total(μl) | | | | 11000 |

2) Pre-read Luminescence at −5 min for background after 2 h incubation.

### 4. Compound Addition and Lum Signal Collection

1) Compounds were diluted with assay buffer to 10 μM top as below:
ECHO-Transferred 180 nL compounds/DMSO to 96-well plate (corning, Cat #651201).
Added 150 μL different pH buffers to compound plate. Mixed well.
2)Removed 80 μL GloSensor from the cell plate, added pH buffer with or without compound, 100 μL/well to cell plate.
3) Transferred the cell plate into plate reader immediately.
4)Read and collected Luminescence signal from 0 min to 30 min on Envision.

The map of compounds plate and pH activation

| | | 1 | 2 | 3 | 4 | 5 | 6 | 7 | 8 | 9 | 10 | 11 | 12 |
|---|---|---|---|---|---|---|---|---|---|---|---|---|---|
| Cpd | A | DMSO | 10000 | 3333 | 1111 | 370.37 | 123.46 | 41.15 | 13.72 | 4.572 | 1.524 | 0.5081 | DMSO |
| 1 | B | (low | 10000 | 3333 | 1111 | 370.37 | 123.46 | 41.15 | 13.72 | 4.572 | 1.524 | 0.5081 | (high |
| Cpd | C | control) | 10000 | 3333 | 1111 | 370.37 | 123.46 | 41.15 | 13.72 | 4.572 | 1.524 | 0.5081 | control) |
| 2 | D | | 10000 | 3333 | 1111 | 370.37 | 123.46 | 41.15 | 13.72 | 4.572 | 1.524 | 0.5081 | |
| Cpd | E | | 10000 | 3333 | 1111 | 370.37 | 123.46 | 41.15 | 13.72 | 4.572 | 1.524 | 0.5081 | |
| 3 | F | | 10000 | 3333 | 1111 | 370.37 | 123.46 | 41.15 | 13.72 | 4.572 | 1.524 | 0.5081 | |
| Cpd | G | | 10000 | 3333 | 1111 | 370.37 | 123.46 | 41.15 | 13.72 | 4.572 | 1.524 | 0.5081 | |
| 4 | H | | 10000 | 3333 | 1111 | 370.37 | 123.46 | 41.15 | 13.72 | 4.572 | 1.524 | 0.5081 | |

10000 nM top, 3-fold dilution, 10 points, in duplicates

High control: pH 6.0+0.1% DMSO
Low control: pH 8.4+0.1% DMSO Compound: pH 6.0 buffer+Compound

### Data Analysis

The percent (%) inhibition at each concentration of compound was calculated based on and relative to the signal in the High and Low control wells contained within each assay plate. The High control wells as 0% inhibition (pH 6.0), and the low control wells as 100% inhibition (pH 8.4).

Inhibition rate of the compound was calculated according to the formula:

$$Inhibition \% = [ CTL pH 6. - CPD pH 6. ] / [ CTL pH 6. - CTL pH 8.4 ] ⋆ 100$$

The concentrations and % inhibition values for tested compounds were plotted and the concentration of compound required for 50% inhibition (IC50) was determined with a four-parameter logistic dose response equation.

The endpoint value (IC50) for the reference peptide/compound was evaluated in each experiment as a quality control measure. If the endpoint value was within 3-fold of the expected value, then the experiment was deemed acceptable.

### Results

The results of the described GPR68 CAMP GloSensor assay, given as IC50 values, indicated the concentration of test compound required for 50% inhibition. The results for this assay for sample compounds of the present disclosure are provided in Table 1 below.

TABLE 1
GPR68 cAMP Glosensor assay results

| Compound number | GPR68 cAMP Glosensor assay IC₅₀ (μM) |
|---|---|
| 1 | 0.593 |
| 2 | 0.861 |
| 3 | 1.2 |
| 4 | 1.29 |
| 5 | 1.41 |
| 6 | 1.68 |
| 7 | 2.02 |
| 8 | 2.08 |
| 9 | 3.46 |
| 10 | 3.82 |
| 11 | 3.83 |
| 12 | 4.49 |
| 13 | 4.81 |
| 14 | 5.72 |
| 15 | 6.68 |
| 16 | 6.9 |
| 17 | 7.02 |
| 18 | 7.39 |
| 19 | 9.7 |
| 23 | 17 |
| 25 | 14.9 |
| 36 | 11.5 |
| 55 | 8.6 |
| 56 | 3.52 |

The present disclosure is further described by reference to the following numbered embodiments.

### Itemised Listing of Embodiments

Embodiment 1. A compound having the structure of formula (I):

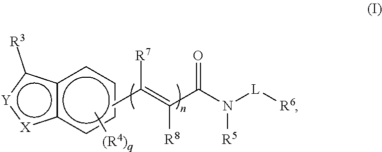

or a pharmaceutically acceptable salt, solvate or stereoisomer thereof,
wherein:
X is selected from N and NR¹;
Y is selected from N and NR²;
R¹ and R² are independently selected from H, C₁₋₁₀ alkyl, C₁₋₁₀ heteroalkyl, C₂₋₁₀ alkenyl, C₂₋₁₀ alkynyl, C₃₋₆ carbocyclyl, heterocyclyl, aryl, and heteroaryl, each of which is optionally substituted with one or more substituents selected from C₁₋₁₀ alkyl, halogen, —CN, —OR⁹, —SR⁹, —S(O)₂R⁹, —NR⁹R¹⁰, —NO₂, ═(O), ═(S), ═(N)R⁹, —(N)R¹⁰, —C(O)R⁹, —C(O)NR⁹R¹⁰, —C(O)SR⁹ or —C(O)OR⁹; or with one or more groups selected from C₃₋₆ carbocyclyl, heterocyclyl, aryl, and heteroaryl, each of which is optionally substituted with one or more substituents selected from C₁₋₁₀ alkyl, halogen, —CN, —OR⁹, —SR⁹, —S(O)₂R⁹, —NR⁹R¹⁰, —NO₂, ═(O), ═(S), —(N)R⁹, ═(N)R¹⁰, —C(O)R⁹, —C(O) NR⁹R¹⁰, —C(O)SR⁹ or —C(O)OR⁹;
or R¹ and R², together with the atoms to which they are attached, form a fused 5-6 membered heteroaryl or a fused 5-6 membered heterocyclyl, optionally substituted with one or more substituents selected from optionally substituted C₁₋₁₀ alkyl, halogen, —CN, —OR⁹, —SR⁹, —S(O)₂R⁹, —NR⁹R¹⁰, —NO₂, ═(O), ═(S), ═(N)R⁹, ═(N)R¹⁰, —C(O)R⁹, —C(O)NR⁹R¹⁰, —C(O)SR⁹ or —C(O)OR⁹;
each R⁹ is independently selected from H, optionally substituted C₁₋₆ alkyl, and optionally substituted C₅₋₆ aryl;
each R¹⁰ is independently selected from H, optionally substituted C₁₋₆ alkyl, optionally substituted C₅₋₆ aryl, and —C(O)R⁹;
R³ is selected from H, halogen, hydroxy, optionally substituted C₁₋₆ alkyl, optionally substituted C₂₋₆ alkenyl, optionally substituted C₂₋₆ alkynyl, optionally substituted C₁₋₆ alkylthio, optionally substituted C₂₋₈ alkoxyalkyl, optionally substituted C₃₋₁₀ carbocyclyl, optionally substituted C₅₋₁₀ aryl, —CN, —OR⁹, —SR⁹, —NR⁹R¹⁰, —C(O)R⁹, —C(O)OR⁹, —S(O)₂R⁹, and —NO₂;
or R³ and R², together with the atoms to which they are attached, form a fused 5-6 membered heteroaryl or a fused 5-6 membered heterocyclyl, optionally substituted with one or more substituents selected from C₁₋₁₀ alkyl, halogen, —CN, —OR⁹, —SR⁹, —S(O)₂R⁹, —NR⁹R¹⁰, —NO₂, ═(O), ═(S), ═(N)R⁹, ═(N)R¹⁰, —C(O)R⁹, —C(O)NR⁹R¹⁰, —C(O)SR⁹ or —C(O)OR⁹;
q is an integer from 0 to 3;
R⁴ is selected from halogen, hydroxy, optionally substituted C₁₋₆ alkyl, optionally substituted C₂₋₆ alkenyl, optionally substituted C₂₋₆ alkynyl, optionally substituted C₁₋₆ alkylthio, optionally substituted C₂₋₈ alkoxyalkyl, optionally substituted C₃₋₁₀ carbocyclyl, optionally substituted C₆₋₁₀ aryl, —CN, —OR⁹, —SR⁹, —NR⁹R¹⁰, —C(O)R⁹, —C(O)OR⁹, —S(O)₂R⁹, and —NO₂;
R⁵ is selected from H, optionally substituted C₁₋₆ alkyl, optionally substituted C₂₋₆ alkenyl, optionally substituted C₂₋₆ alkynyl, optionally substituted C₂₋₈ alkoxyalkyl, optionally substituted C₃₋₆ carbocyclyl, optionally substituted C₅₋₁₀ aryl, optionally substituted 5-6 membered heteroaryl, and optionally substituted 5-6 membered heterocyclyl;
L is a bond, or a linker selected from —C(O)N(R⁹)—, —S(O)₂—, —C(O)—, or —C(O)O—; or from optionally substituted C₁₋₄ alkylene, optionally substituted C₂₋₄ alkenylene, —OC₁₋₄ alkylene, or —OC₂₋₄ alkenylene, each of which is optionally interrupted with —O—, —S—, —NR⁹—, —NR¹⁰—, —C(O)N(R⁹)—, —N(R⁹) C(O)—, —S(O)₂—, —N(R⁹) S(O)₂—, —S(O)₂N(R⁹)—, —C(O)—, —C(O)O—, or —O(O)C—;
R⁶ is selected from C₅₋₁₀ aryl, 5-10 membered heteroaryl, 3-10 membered heterocyclyl, C₃₋₁₀ carbocyclyl, C₁₋₁₀ alkyl, C₂₋₁₀ alkenyl, or C₂₋₁₀ alkynyl, each optionally substituted with one or more substituents optionally selected from optionally substituted C₁₋₆ alkyl, optionally substituted C₅₋₆ aryl, optionally substituted 5-6 membered heteroaryl, optionally substituted 5-6 membered heterocyclyl, halogen, —CN, —OR¹¹, —SR¹¹, —S(O)₂R¹¹, —S(O)₂N(R¹¹)₂, —C(O)N(R¹¹)₂, —NR¹¹C(O)R¹¹, —N(R¹¹) S(O)₂R¹¹, —N(R¹¹)₂, —NO₂,═(O),═(S), ═(N)R¹¹, —C(O)R¹¹, —C(O)OR¹¹, —C(O) N(R¹¹)₂, or —C(O)SR¹¹; wherein two adjacent optional substituents, when present, together with the atoms to which they are attached, may form an optionally substituted fused 5-6 membered heteroaryl or an optionally substituted fused 5-6 membered heterocyclyl;
or R⁶ and R⁵, together with the atoms to which they are attached, form a 3-10 membered heterocyclyl or 5-10 membered heteroaryl, optionally substituted with one or more substituents optionally selected from optionally substituted C₁₋₁₀ alkyl, optionally substituted C₂₋₆ alkenyl, optionally substituted C₂₋₆ alkynyl, halogen, —CN, —OR¹¹, —SR¹¹, —S(O)₂R¹¹, —S(O)₂N(R¹¹)₂, —C(O)N(R¹¹)₂, —NR¹¹C(O)R¹¹, —N(R¹¹) S(O)₂R¹¹, —N(R¹¹)₂, —NO₂,═(O),═(S), ═(N)R¹¹, —C(O)R¹¹, —C(O)OR¹¹, —C(O) N(R¹¹)₂, or —C(O)SR¹¹;
each R¹¹ is independently selected from H, optionally substituted C₁₋₆ alkyl, optionally substituted C₂₋₆ alkenyl, optionally substituted C₂₋₆ alkynyl, optionally substituted C₅₋₁₀ aryl, optionally substituted C₂₋₂₀ alkoxyalkyl, C₁-C₆ haloalkyl, —C(O)R⁹, —OR⁹, —S(O)₂R⁹, and —S(O)₂N(R⁹) 2.
R⁷ and R⁸ are independently selected from H, C₁₋₆ alkyl, C₅₋₁₀ aryl, 5-10 membered heteroaryl, and C₃₋₁₀ carbocyclyl, each optionally substituted with one or more substituents selected from C₁₋₆ alkyl, C₅₋₆ aryl, halogen, —CN, —OR⁹, —SR⁹, —S(O)₂R⁹, —NR⁹R¹⁰, —NO₂,═(O), ═(S), ═(N)R⁹, ═(N)R¹⁰, —C(O)R⁹, —C(O)NR⁹R¹⁰, —C(O)SR⁹ or —C(O)OR⁹;
n is an integer from 1 to 4.

Embodiment 2. The compound according to embodiment 1, having the structure of Formula (Ia):

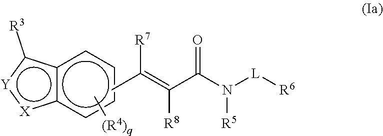

or a pharmaceutically acceptable salt, solvate or stereoisomer thereof.

Embodiment 3. The compound according to embodiment 1 or embodiment 2, having the structure of Formula (II):

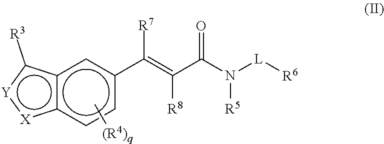

or a pharmaceutically acceptable salt, solvate or stereoisomer thereof.

Embodiment 4. The compound according to any one of embodiments 1 to 3, having the structure of Formula (IIa):

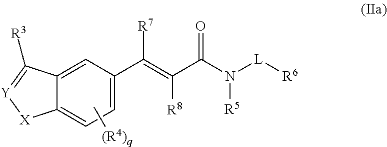

or a pharmaceutically acceptable salt, solvate or stereoisomer thereof.

Embodiment 5. The compound according to any one of embodiments 1 to 4, having the structure of Formula (III):

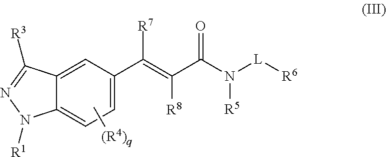

or a pharmaceutically acceptable salt, solvate or stereoisomer thereof.

Embodiment 6. The compound according to any one of embodiments 1 to 5, wherein R¹ is C₁₋₁₀ alkyl, C₁₋₁₀ heteroalkyl, C₁-6 carbocyclyl, or heterocyclyl, each of which is optionally substituted with one or more substituents selected from halogen, —CN, —OR⁹, —SR⁹, —S(O)₂R⁹, —NR⁹R¹⁰, —NO₂, ═(O), ═(S), ═(N)R⁹, ═(N)R¹⁰, —C(O)R⁹, —C(O) NR⁹R¹⁰, —C(O)SR⁹ or —C(O)OR⁹; or with one or more groups selected from C1-6 carbocyclyl, heterocyclyl, and heteroaryl, each of which is optionally substituted with one or more substituents selected from halogen, —CN, —OR⁹, —SR⁹, —S(O)₂R⁹, —NR⁹R¹⁰, —NO₂,═(O),═(S), ═(N)R⁹, ═(N)R¹⁰, —C(O)R⁹, —C(O)NR⁹R¹⁰, —C(O)SR⁹ or —C(O)OR⁹.

Embodiment 7. The compound according to any one of embodiments 1 to 6, wherein R¹ is C₁₋₆ alkyl, optionally substituted with one or more substituents selected from halogen, —CN, —OR⁹, —SR⁹, —S(O)₂R⁹, —NR⁹R¹⁰, —NO₂, ═(O), ═(S), ═(N)R⁹, ═(N)R¹⁰, —C(O)R⁹, —C(O)NR⁹R¹⁰, —C(O)SR⁹ or —C(O)OR⁹; or with one or more groups selected from C₁₋₆ carbocyclyl, heterocyclyl, and heteroaryl, each of which is optionally substituted with one or more substituents selected from halogen, —CN, —OR⁹, —SR⁹, —S(O)₂R⁹, —NR⁹R¹⁰, —NO₂, ═(O), ═(S), ═(N)R⁹, ═(N)R¹⁰, —C(O)R⁹, —C(O)NR⁹R¹⁰, —C(O)SR⁹ or —C(O)OR⁹.

Embodiment 8. The compound according to any one of embodiments 1 to 7, wherein R¹ is C₁₋₆ alkyl.

Embodiment 9. The compound according to any one of embodiments 1 to 8, wherein R¹ is methyl, ethyl, propyl or isopropyl.

Embodiment 10. The compound according to any one of embodiments 1 to 9, wherein R¹ is methyl.

Embodiment 11. The compound according to any one of embodiments 1 to 8, wherein R¹ is C₁₋₆ alkyl substituted with —OR⁹, —C(O)R⁹, —C(O)NR⁹R¹⁰, C(O)SR⁹ or —C(O)OR⁹.

Embodiment 12. The compound according to embodiment 11, wherein R⁹ is methyl.

Embodiment 13. The compound according to any one of embodiments 1 to 8, wherein R¹ is C₁₋₆ alkyl substituted with halogen or —CN, optionally wherein halogen is F.

Embodiment 14. The compound according to any one of embodiments 1 to 8, wherein R¹ is C₁₋₆ alkyl substituted with heteroaryl, which is optionally substituted with one or more substituents selected from halogen, —CN, —OR⁹, —SR⁹, —S(O)₂R⁹, —NR⁹R¹⁰, —NO₂, ═(O),═(S), ═(N)R⁹, —(N)R¹⁰, —C(O)R⁹, —C(O)NR⁹R¹⁰, —C(O)SR⁹ or —C(O)OR⁹.

Embodiment 15. The compound according to embodiment 14, wherein R¹ is an optionally substituted 5-membered heteroaryl, wherein the 5-membered heteroaryl is selected from furyl, thienyl, pyrrolyl, oxazolyl, thiazolyl, imidazolyl, pyrazolyl, isoxazolyl, isothiazolyl, triazolyl, and thiadiazolyl, each of which may be optionally substituted.

Embodiment 16. The compound according to any one of embodiments 1 to 8, wherein R¹ is C₁₋₆ alkyl substituted with heterocyclyl, wherein the heterocyclyl is optionally substituted with one or more substituents selected from halogen, —CN, —OR⁹, —SR⁹, —S(O)₂R⁹, —NR⁹R¹⁰, —NO₂, —(O), —(S), —(N)R⁹, —(N)R¹⁰, —C(O)R⁹, —C(O) NR⁹R¹⁰, —C(O)SR⁹ or —C(O)OR⁹.

Embodiment 17. The compound according to embodiment 16, wherein the heterocyclyl is an optionally substituted 3- to 6-membered heterocyclyl.

Embodiment 18. The compound according to embodiment 16 or embodiment 17, wherein the heterocyclyl is an optionally substituted oxygen heterocycle.

Embodiment 19. The compound according to embodiment 18, wherein the oxygen heterocycle is selected from:

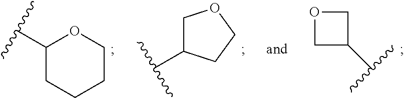 [SMILES: *C1CCCCO1 ; *C1CCOC1 ; *C1COC1]

each of which may be optionally substituted.

Embodiment 20. The compound according to any one of embodiments 1 to 8, wherein R¹ is C₁₋₆ alkyl substituted with C₁₋₆ carbocyclyl, which is optionally substituted with one or more substituents selected from halogen, —CN, —OR⁹, —SR⁹, —S(O)₂R⁹, —NR⁹R¹⁰, —NO₂, ═(O), ═(S), ═(N)R⁹, —(N)R¹⁰, —C(O)R⁹, —C(O)NR⁹R¹⁰, —C(O)SR⁹ or —C(O)OR⁹.

Embodiment 21. The compound according to embodiment 20, wherein the C₁₋₆ carbocyclyl is an optionally substituted cyclopropyl.

Embodiment 22. The compound according to embodiment 20 or 21, wherein R¹ is

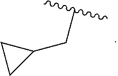 [SMILES: *CC1CC1]

Embodiment 23. The compound according to any one of embodiments 1 to 22, wherein R⁷ and R⁸ are H, and having the structure of Formula (IV):

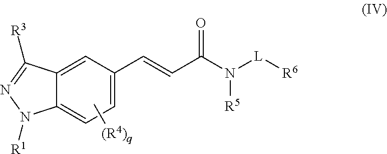

or a pharmaceutically acceptable salt, solvate or stereoisomer thereof.

Embodiment 24. The compound according to any one of embodiments 1 to 23, wherein R³ is H.

Embodiment 25. The compound according to any one of embodiments 1 to 24, wherein q is 0, 1 or 2.

Embodiment 26. The compound according to any one of embodiments 1 to 25, having the structure of Formula (Va), (Vb) or (Vc):

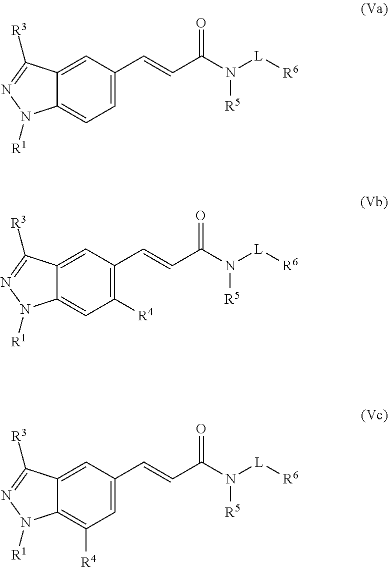

or a pharmaceutically acceptable salt, solvate or stereoisomer thereof.

Embodiment 27. The compound according to any one of embodiments 1 to 26, wherein R⁴ is selected from halogen, hydroxy, optionally substituted C₂₋₈ alkoxyalkyl, —CN, —OR⁹, —SR⁹, —NR⁹R¹⁰, —C(O)R⁹, —C(O)OR⁹, —S(O)₂R⁹, and —NO₂.

Embodiment 28. The compound according to any one of embodiments 1 to 27, wherein R⁴ is selected from halogen, —CN, and —NO₂.

Embodiment 29. The compound according to any one of embodiments 1 to 28, wherein R⁴ is a halogen, preferably F.

Embodiment 30. The compound according to any one of embodiments 1 to 29, wherein L is a linker selected from —S(O) 2—, optionally substituted C₁₋₄ alkylene, or C2-4 alkenylene.

Embodiment 31. The compound according to any one of embodiments 1 to 30, wherein L is a bond, and having the structure of Formula (VI):

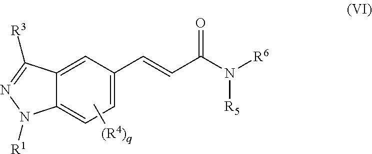

or a pharmaceutically acceptable salt, solvate or stereoisomer thereof.

Embodiment 32. The compound according to embodiment 31, having the structure of Formula (VIa), (VIb) or (VIc):

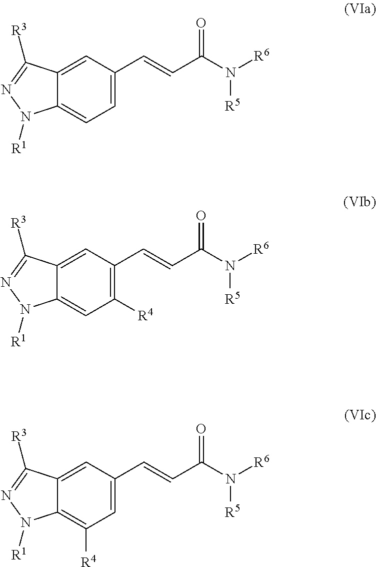

or a pharmaceutically acceptable salt, solvate or stereoisomer thereof.

Embodiment 33. The compound according to any one of embodiments 1 to 32, wherein R⁵ is H, C₁-6 alkyl, or C₂-8 alkoxyalkyl.

Embodiment 34. The compound according to any one of embodiments 1 to 33, wherein R⁵ is H.

Embodiment 35. The compound according to any one of embodiments 1 to 34, wherein R⁶ is selected from C₆₋₁₀ aryl or 5-6 membered heteroaryl, each optionally substituted with one or more substituents optionally selected from optionally substituted C₁₋₆ alkyl, optionally substituted C₁₋₆ aryl, optionally substituted 5-6 membered heteroaryl, optionally substituted 5-6 membered heterocyclyl, halogen, —CN, —OR¹¹, —SR¹¹, —S(O)₂R¹¹, —S(O)₂N(R¹¹)₂, —S(O)(NR¹⁰)R¹¹, —C(O)N(R¹¹)₂, —NR¹¹C(O)R¹¹, —N(R¹¹) S(O)₂R¹¹, —N(R¹¹)₂, —NO₂,═(O), ═(S), ═(N)R¹¹, —C(O)R¹¹, —C(O)OR¹¹, —C(O)N(R¹¹)₂, or —C(O)SR¹¹; wherein two adjacent optional substituents, when present, together with the atoms to which they are attached, may form an optionally substituted fused 5-6 membered heteroaryl or an optionally substituted fused 5-6 membered heterocyclyl.

Embodiment 36. The compound according to any one of embodiments 1 to 35, wherein the optional substituent of R⁶ is one or more substituents selected from halogen, optionally substituted 5 membered heterocyclyl, optionally substituted 5 membered heteroaryl, —C(O)OR¹¹, —N(R¹¹) S(O)₂R¹¹, —C(O)N(R¹¹)₂, and —S(O)₂N(R¹¹) 2.

Embodiment 37. The compound according to any one of embodiments 1 to 36, wherein R⁶ is selected from C6 aryl, 5 membered heteroaryl, or 6 membered heteroaryl, each of which may be optionally substituted.

Embodiment 38. The compound according to any one of embodiments 1 to 37, wherein R⁶ is selected from:

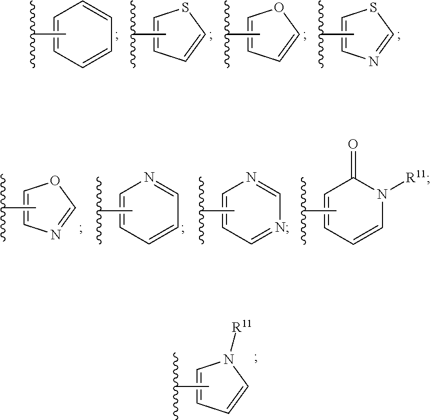

each of which may be optionally substituted.

Embodiment 39. The compound according to any one of embodiments 35 to 38, selected from the group consisting of:

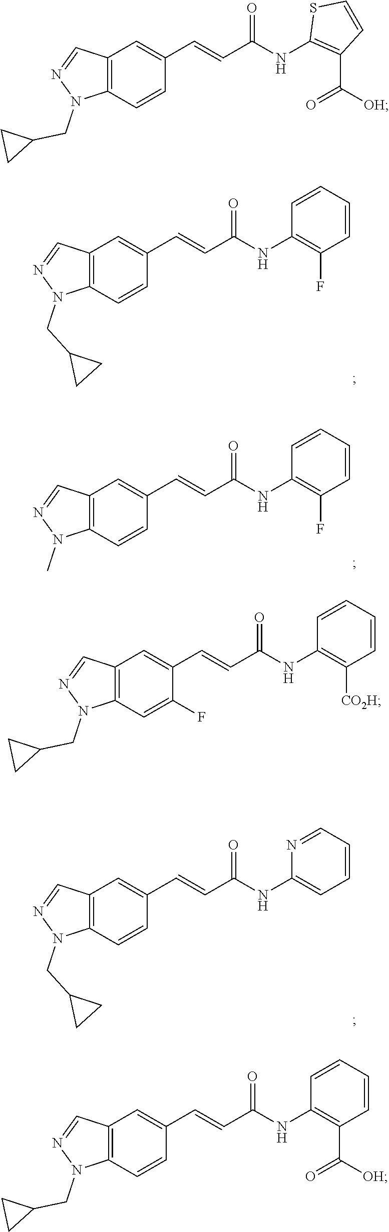 [SMILES: O=C(/C=C/c1ccc2c(cnn2CC2CC2)c1)Nc1sccc1C(=O)O ; O=C(/C=C/c1ccc2c(cnn2CC2CC2)c1)Nc1ccccc1F ; Cn1ncc2cc(/C=C/C(=O)Nc3ccccc3F)ccc21 ; O=C(/C=C/c1cc2cnn(CC3CC3)c2cc1F)Nc1ccccc1C(=O)O ; O=C(/C=C/c1ccc2c(cnn2CC2CC2)c1)Nc1ccccn1 ; O=C(/C=C/c1ccc2c(cnn2CC2CC2)c1)Nc1ccccc1C(=O)O]

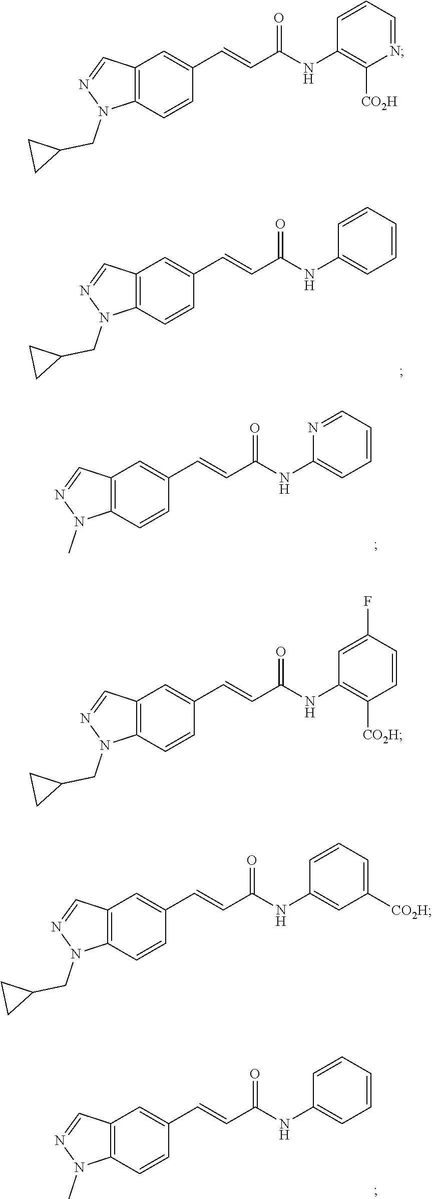 [SMILES: O=C(/C=C/c1ccc2c(cnn2CC2CC2)c1)Nc1cccnc1C(=O)O ; O=C(/C=C/c1ccc2c(cnn2CC2CC2)c1)Nc1ccccc1 ; Cn1ncc2cc(/C=C/C(=O)Nc3ccccn3)ccc21 ; O=C(/C=C/c1ccc2c(cnn2CC2CC2)c1)Nc1cc(F)ccc1C(=O)O ; O=C(/C=C/c1ccc2c(cnn2CC2CC2)c1)Nc1cccc(C(=O)O)c1 ; Cn1ncc2cc(/C=C/C(=O)Nc3ccccc3)ccc21]

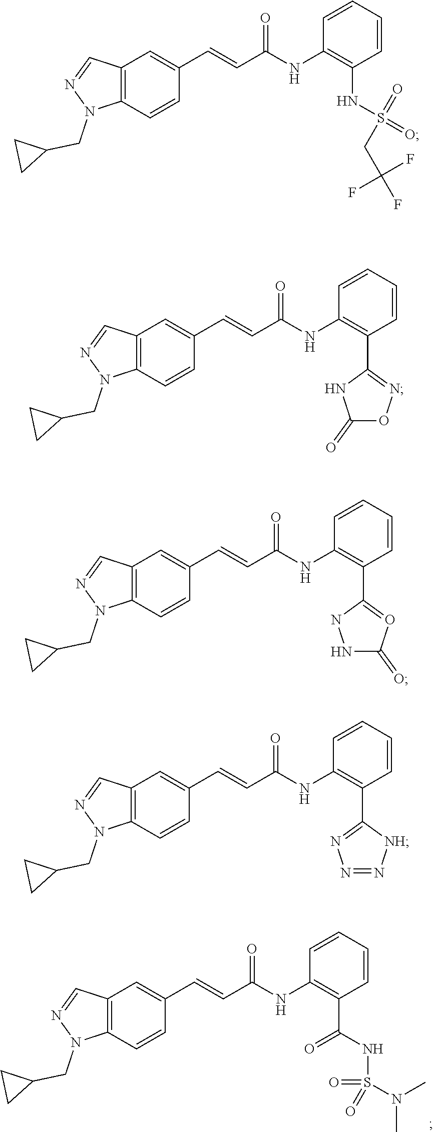

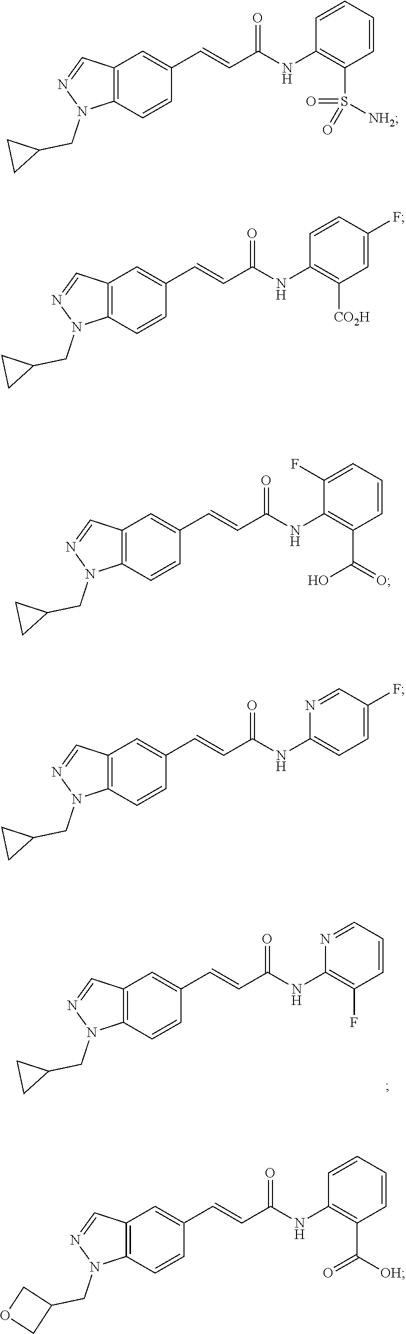 [SMILES: NS(=O)(=O)c1ccccc1NC(=O)/C=C/c1ccc2c(cnn2CC2CC2)c1 ; O=C(/C=C/c1ccc2c(cnn2CC2CC2)c1)Nc1ccc(F)cc1C(=O)O ; O=C(/C=C/c1ccc2c(cnn2CC2CC2)c1)Nc1c(F)cccc1C(=O)O ; O=C(/C=C/c1ccc2c(cnn2CC2CC2)c1)Nc1ccc(F)cn1 ; O=C(/C=C/c1ccc2c(cnn2CC2CC2)c1)Nc1ncccc1F ; O=C(/C=C/c1ccc2c(cnn2CC2COC2)c1)Nc1ccccc1C(=O)O]

or a pharmaceutically acceptable salt, solvate or stereoisomer thereof.

Embodiment 40. The compound according to any one of embodiments 35 to 39, selected from the group consisting of:

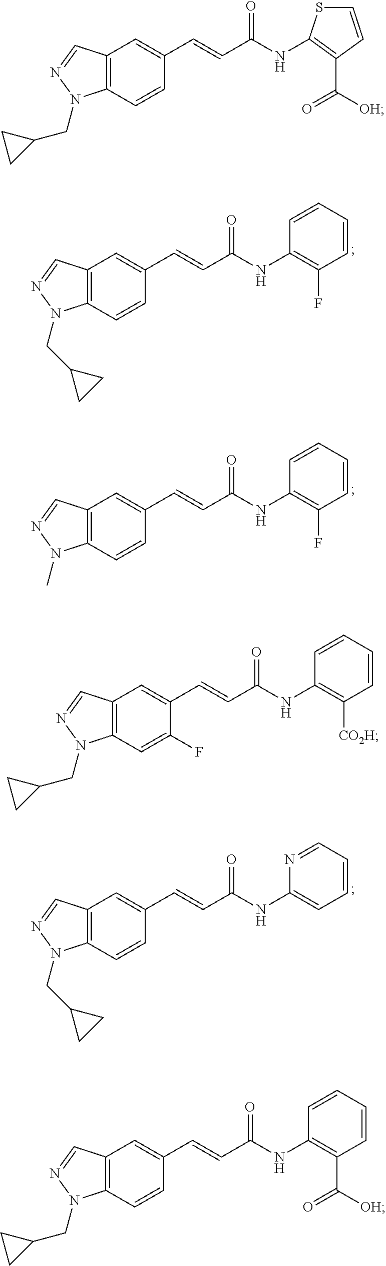 [SMILES: O=C(/C=C/c1ccc2c(cnn2CC2CC2)c1)Nc1sccc1C(=O)O ; O=C(/C=C/c1ccc2c(cnn2CC2CC2)c1)Nc1ccccc1F ; Cn1ncc2cc(/C=C/C(=O)Nc3ccccc3F)ccc21 ; O=C(/C=C/c1cc2cnn(CC3CC3)c2cc1F)Nc1ccccc1C(=O)O ; O=C(/C=C/c1ccc2c(cnn2CC2CC2)c1)Nc1ccccn1 ; O=C(/C=C/c1ccc2c(cnn2CC2CC2)c1)Nc1ccccc1C(=O)O]

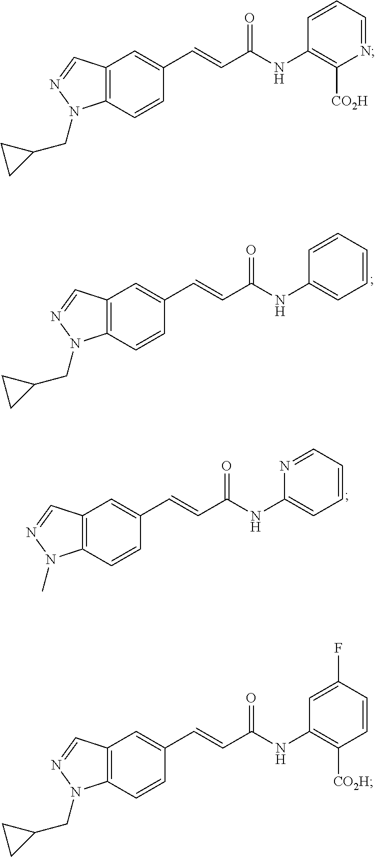 [SMILES: O=C(/C=C/c1ccc2c(cnn2CC2CC2)c1)Nc1cccnc1C(=O)O ; O=C(/C=C/c1ccc2c(cnn2CC2CC2)c1)Nc1ccccc1 ; Cn1ncc2cc(/C=C/C(=O)Nc3ccccn3)ccc21 ; O=C(/C=C/c1ccc2c(cnn2CC2CC2)c1)Nc1cc(F)ccc1C(=O)O]

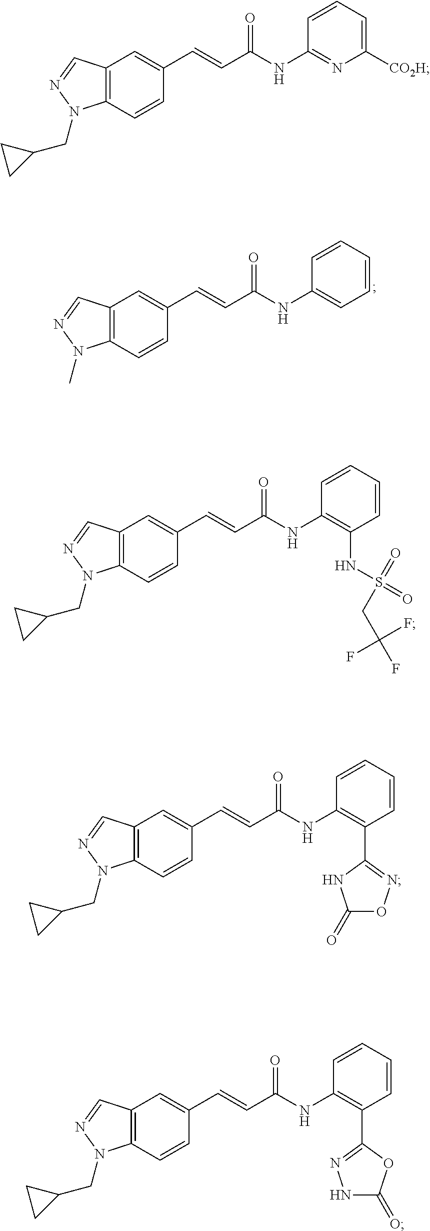 [SMILES: O=C(/C=C/c1ccc2c(cnn2CC2CC2)c1)Nc1cccc(C(=O)O)n1 ; Cn1ncc2cc(/C=C/C(=O)Nc3ccccc3)ccc21 ; O=C(/C=C/c1ccc2c(cnn2CC2CC2)c1)Nc1ccccc1NS(=O)(=O)CC(F)(F)F ; O=C(/C=C/c1ccc2c(cnn2CC2CC2)c1)Nc1ccccc1-c1noc(=O)[nH]1 ; O=C(/C=C/c1ccc2c(cnn2CC2CC2)c1)Nc1ccccc1-c1n[nH]c(=O)o1]

or a pharmaceutically acceptable salt, solvate or stereoisomer thereof.

Embodiment 41. The compound according to any one of embodiments 35 to 40, selected from the group consisting of:

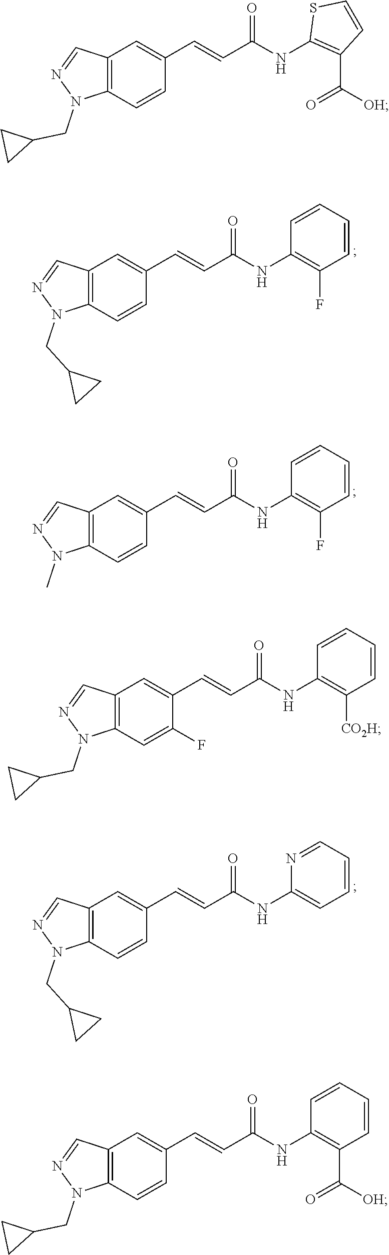 [SMILES: O=C(/C=C/c1ccc2c(cnn2CC2CC2)c1)Nc1sccc1C(=O)O ; O=C(/C=C/c1ccc2c(cnn2CC2CC2)c1)Nc1ccccc1F ; Cn1ncc2cc(/C=C/C(=O)Nc3ccccc3F)ccc21 ; O=C(/C=C/c1cc2cnn(CC3CC3)c2cc1F)Nc1ccccc1C(=O)O ; O=C(/C=C/c1ccc2c(cnn2CC2CC2)c1)Nc1ccccn1 ; O=C(/C=C/c1ccc2c(cnn2CC2CC2)c1)Nc1ccccc1C(=O)O]

or a pharmaceutically acceptable salt, solvate or stereoisomer thereof.

Embodiment 42. The compound according to any one of embodiments 1 to 34, wherein R⁶ is selected from 3-10 membered heterocyclyl and C₃₋₁₀ carbocyclyl, each optionally substituted with one or more substituents optionally selected from optionally substituted C₁₋₆ alkyl, optionally substituted C₁₋₆ aryl, optionally substituted 5-6 membered heteroaryl, optionally substituted 5-6 membered heterocyclyl, halogen, —CN, —OR¹¹, —SR¹¹, —S(O)₂R¹¹, —S(O)₂N(R¹¹)₂, —S(O)(NR¹⁰)R¹¹, —C(O)N(R¹¹)₂, —NR¹¹C(O)R¹¹, —N(R¹¹) S(0) 2R¹¹, —N(R¹¹)₂, —NO₂,═(O),═(S), ═(N)R¹¹, —C(O)R¹¹, —C(O)OR¹¹, —C(O)N(R¹¹)₂, or —C(O)SR¹¹.

Embodiment 43. The compound according to embodiment 42, wherein the optional substituent of R⁶ is one or more substituents selected from halogen and —C(O)OR¹¹, wherein R¹¹ is H or optionally substituted C₁₋₆ alkyl.

Embodiment 44. The compound according to embodiment 42 or embodiment 43, selected from the group consisting of:

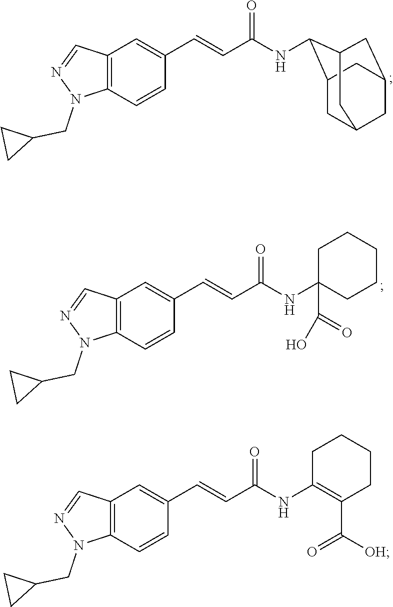 [SMILES: O=C(/C=C/c1ccc2c(cnn2CC2CC2)c1)NC1C2CC3CC(C2)CC1C3 ; O=C(/C=C/c1ccc2c(cnn2CC2CC2)c1)NC1(C(=O)O)CCCCC1 ; O=C(/C=C/c1ccc2c(cnn2CC2CC2)c1)NC1=C(C(=O)O)CCCC1]

or a pharmaceutically acceptable salt, solvate or stereoisomer thereof.

Embodiment 45. The compound according to any one of embodiments 1 to 34, wherein R⁶ and R⁵, together with the atoms to which they are attached, form a 3-10 membered heterocyclyl or 5-10 membered heteroaryl, optionally substituted with one or more substituents optionally selected from optionally substituted C₁₋₁₀ alkyl, optionally substituted C₂₋₆ alkenyl, optionally substituted C₂₋₆ alkynyl, halogen, —CN, —OR¹¹, —SR¹¹, —S(O)₂R¹¹, —S(O)₂N(R¹¹)₂, —S(O)(NR¹⁰)R¹¹, —C(O)N(R¹¹)₂, —NR¹¹C(O)R¹¹, —N(R¹¹) S(O)₂R¹¹, —N(R¹¹)₂, —NO₂,═(O),═(S), ═(N)R¹¹, —C(O)R¹¹, —C(O)OR¹¹, —C(O)N(R¹¹)₂, or —C(O)SR¹¹.

Embodiment 46. The compound according to embodiment 45, wherein the optional substituent of R⁶ is one or more substituents selected halogen from —C(O)OR¹¹, wherein R¹¹ is H or optionally substituted C₁₋₆ alkyl.

Embodiment 47. The compound according to embodiment 45 or

embodiment 46, having the structure:

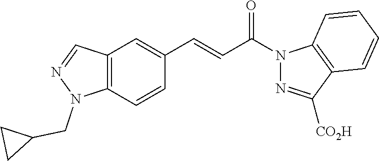 [SMILES: O=C(O)c1nn(C(=O)/C=C/c2ccc3c(cnn3CC3CC3)c2)c2ccccc12]

or a pharmaceutically acceptable salt, solvate or stereoisomer thereof.

Embodiment 48. A compound having the structure:

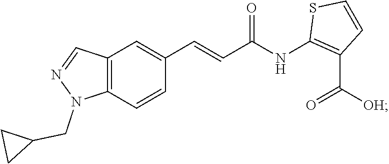 [SMILES: O=C(/C=C/c1ccc2c(cnn2CC2CC2)c1)Nc1sccc1C(=O)O]

or a pharmaceutically acceptable salt thereof.

Embodiment 49. The compound or pharmaceutically acceptable salt thereof according to embodiment 48, wherein the compound is:

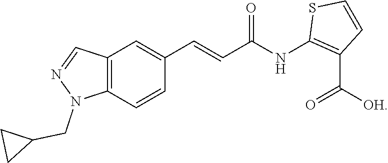 [SMILES: O=C(/C=C/c1ccc2c(cnn2CC2CC2)c1)Nc1sccc1C(=O)O]

Embodiment 50. A compound having the structure:

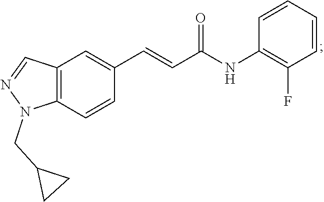 [SMILES: O=C(/C=C/c1ccc2c(cnn2CC2CC2)c1)Nc1ccccc1F]

or a pharmaceutically acceptable salt thereof.

Embodiment 51. The compound or pharmaceutically acceptable salt thereof according to embodiment 50, wherein the compound is:

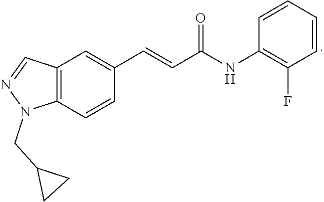 [SMILES: O=C(/C=C/c1ccc2c(cnn2CC2CC2)c1)Nc1ccccc1F]

Embodiment 52. A compound having the structure:

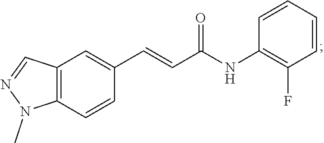 [SMILES: Cn1ncc2cc(/C=C/C(=O)Nc3ccccc3F)ccc21]

or a pharmaceutically acceptable salt thereof.

Embodiment 53. The compound or pharmaceutically acceptable salt thereof according to embodiment 52, wherein the compound is:

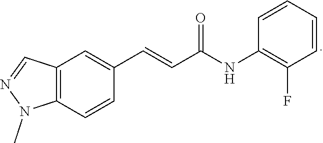 [SMILES: Cn1ncc2cc(/C=C/C(=O)Nc3ccccc3F)ccc21]

Embodiment 54. A compound having the structure:

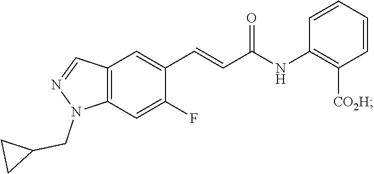 [SMILES: O=C(/C=C/c1cc2cnn(CC3CC3)c2cc1F)Nc1ccccc1C(=O)O]

or a pharmaceutically acceptable salt thereof.

Embodiment 55. The compound or pharmaceutically acceptable salt thereof according to embodiment 54, wherein the compound is:

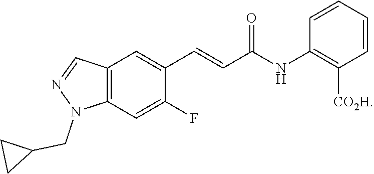 [SMILES: O=C(/C=C/c1cc2cnn(CC3CC3)c2cc1F)Nc1ccccc1C(=O)O]

Embodiment 56. A compound having the structure:

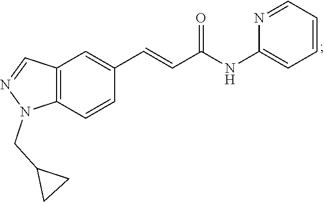 [SMILES: O=C(/C=C/c1ccc2c(cnn2CC2CC2)c1)Nc1ccccn1]

or a pharmaceutically acceptable salt thereof.

Embodiment 57. The compound or pharmaceutically acceptable salt thereof according to embodiment 56, wherein the compound is:

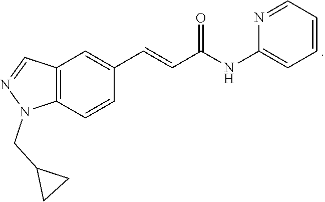 [SMILES: O=C(/C=C/c1ccc2c(cnn2CC2CC2)c1)Nc1ccccn1]

Embodiment 58. A compound having the structure:

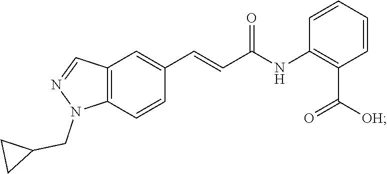 [SMILES: O=C(/C=C/c1ccc2c(cnn2CC2CC2)c1)Nc1ccccc1C(=O)O]

or a pharmaceutically acceptable salt thereof.

Embodiment 59. The compound or pharmaceutically acceptable salt thereof according to embodiment 58, wherein the compound is:

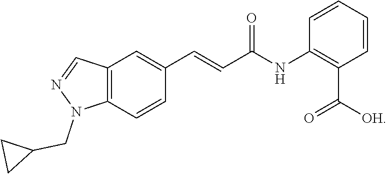 [SMILES: O=C(/C=C/c1ccc2c(cnn2CC2CC2)c1)Nc1ccccc1C(=O)O]

Embodiment 60. A pharmaceutical composition comprising a compound according to any one of embodiments 1 to 59, or a pharmaceutically acceptable salt, solvate or stereoisomer thereof, and a pharmaceutically acceptable excipient.

Embodiment 61. A method of treating and/or preventing a disease, disorder or condition mediated by a proton-activated GPCR in a subject in need thereof, comprising administering to the subject an effective amount of a compound according to any one of embodiments 1 to 59, or a pharmaceutically acceptable salt, solvate or stereoisomer thereof, or a pharmaceutical composition according to embodiment 60.

Embodiment 62. The method according to embodiment 61, wherein the proton-activated GPCR is a GPR68 (OGR1).

Embodiment 63. The method according to embodiment 61 or embodiment 62, wherein the disease, disorder or condition is selected from the group consisting of: an inflammatory disease, disorder or condition; a fibrotic disease, disorder or condition; a proliferative disease, disorder or condition, or a cancer.

Embodiment 64. The method according to embodiment 63, wherein the disease, disorder or condition is an inflammatory disease, disorder or condition.

Embodiment 65. The method according to embodiment 64, wherein the inflammatory disease, disorder or condition is selected from focal segmental glomerulosclerosis, lupus nephritis, membranous nephropathy, IgA nephropathy, chronic obstructive pulmonary disorder (COPD), asthma, and cystic fibrosis.

Embodiment 66. The method according to embodiment 64, wherein the inflammatory disease, disorder or condition is an autoimmune disease, disorder or condition.

Embodiment 67. The method according to embodiment 66, wherein the autoimmune disease, disorder or condition is selected from systemic lupus erythematosus (SLE), inflammatory bowel disease, Crohn's disease, ulcerative colitis, graft versus host disease, multiple sclerosis, and rheumatoid arthritis.

Embodiment 68. The method according to embodiment 63, wherein the disease, disorder or condition is a fibrotic disease, disorder or condition.

Embodiment 69. The method according to embodiment 68, wherein the fibrotic disease, disorder or condition is selected from renal fibrosis, pulmonary fibrosis, cardiovascular fibrosis, ocular fibrosis, dermal fibrosis, pancreatic fibrosis, and liver fibrosis.

Embodiment 70. The method according to embodiment 68, wherein the fibrotic disease, disorder or condition is selected from diabetic cardiomyopathy, congestive heart failure, ischemic heart disease, hypertension, peripheral artery disease, cerebrovascular disease, chronic kidney disease, diabetic nephropathy, systemic sclerosis (scleroderma), hypertrophic scars, keloids, idiopathic pulmonary fibrosis (IPF), non-alcoholic steatohepatitis (NASH), non-alcoholic fatty liver disease (NAFLD), primary biliary cirrhosis (PBC), and primary sclerosis cholangitis (PSC).

Embodiment 71. The method according to embodiment 63, wherein the disease, disorder or condition is a proliferative disease, disorder or condition.

Embodiment 72. The method according to embodiment 71, wherein the proliferative disease, disorder or condition is selected from diabetic retinopathy, diabetic macular edema, macular degeneration, retinopathy of prematurity (ROP), and proliferative vitreoretinopathy (PVR).

Embodiment 73. The method according to embodiment 63, wherein the disease, disorder or condition is a cancer.

Embodiment 74. The method according to embodiment 73, wherein the cancer is selected from a breast cancer, skin cancer (melanoma), pancreatic cancer, lung cancer, prostate cancer, colon cancer, liver cancer (hepatocellular carcinoma), head and neck cancer, ovarian cancer and kidney cancer.

Embodiment 75. The method according to any one of embodiments 61 to 74, wherein the subject is a human.

Embodiment 76. A method of inhibiting a proton-activated GPCR in a cell population, comprising administering to the cell population an effective amount of a compound according to any one of embodiments 1 to 59, or a pharmaceutically acceptable salt, solvate or stereoisomer thereof, or a pharmaceutical composition according to embodiment 60.

Embodiment 77. The method according to embodiment 76, wherein the proton-activated GPCR is a GPR68 (OGR1).

Embodiment 78. The method according to embodiment 76 or embodiment 77, wherein the cell is a tumour cell.

Embodiment 79. The steps, features, integers, compositions and/or compounds disclosed herein or indicated in the specification of this application individually or collectively, and any and all combinations of two or more of said steps or features.

1. A compound having the structure of Formula (II): 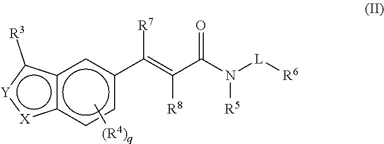
or a pharmaceutically acceptable salt, solvate or stereoisomer thereof,
wherein:
X is selected from N and NR¹;
Y is selected from N and NR²;
R¹ and R² are independently selected from H, C₁₋₁₀ alkyl, C₁₋₁₀ heteroalkyl, C₂₋₁₀ alkenyl, C₂₋₁₀ alkynyl, C₃₋₆ carbocyclyl, heterocyclyl, aryl, and heteroaryl, each of which is optionally substituted with one or more substituents selected from C₁₋₁₀ alkyl, halogen, —CN, —OR⁹, —SR⁹, —S(O)₂R⁹, —NR⁹R¹⁰, —NO₂, ═(O), ═(S), ═(N)R⁹, ═(N)R¹⁰, —C(O)R⁹, —C(O)NR⁹R¹⁰, —C(O)SR⁹ or —C(O)OR⁹; or with one or more substituents selected from C₃₋₆ carbocyclyl, heterocyclyl, aryl, and heteroaryl, each of which is optionally substituted with one or more substituents selected from C₁₋₁₀ alkyl, halogen, —CN, —OR⁹, —SR⁹, —S(O)₂R⁹, —NR⁹R¹⁰, —NO₂, ═(O), ═(S), ═(N)R⁹, ═(N)R¹⁰, —C(O)R⁹, —C(O)NR⁹R¹⁰, —C(O)SR⁹ or —C(O)OR⁹;
or R¹ and R², together with the atoms to which they are attached, form a fused 5-6 membered heteroaryl or a fused 5-6 membered heterocyclyl, each of which is optionally substituted with one or more substituents selected from optionally substituted C₁₋₁₀ alkyl, halogen, —CN, —OR⁹, —SR⁹, —S(O)₂R⁹, —NR⁹R¹⁰, —NO₂, —(O), ═(S), ═(N)R⁹, ═(N)R¹⁰, —C(O)R⁹, —C(O)NR⁹R¹⁰, —C(O)SR⁹ or —C(O)OR⁹;
each R⁹ is independently selected from H, optionally substituted C₁₋₆ alkyl, and optionally substituted C₅₋₆ aryl;
each R¹⁰ is independently selected from H, optionally substituted C₁₋₆ alkyl, optionally substituted C₅₋₆ aryl, and —C(O)R⁹;
R³ is selected from H, halogen, hydroxy, optionally substituted C₁₋₆ alkyl, optionally substituted C₂₋₆ alkenyl, optionally substituted C₂₋₆ alkynyl, optionally substituted C₁₋₆ alkylthio, optionally substituted C₂₋₈ alkoxyalkyl, optionally substituted C₃₋₁₀ carbocyclyl, optionally substituted C₅₋₁₀ aryl, —CN, —OR⁹, —SR⁹, —NR⁹R¹⁰, —C(O)R⁹, —C(O)OR⁹, —S(O)₂R⁹, and —NO₂;
or R³ and R², together with the atoms to which they are attached, form a fused 5-6 membered heteroaryl or a fused 5-6 membered heterocyclyl, each of which is optionally substituted with one or more substituents selected from C₁₋₁₀ alkyl, halogen, CN, —OR⁹, —SR⁹, —S(O)₂R⁹, —NR⁹R¹⁰, —NO₂, —(O), —(S), —(N)R⁹, —(N)R¹⁰, —C(O)R⁹, —C(O)NR⁹R¹⁰, —C(O)SR⁹ or —C(O)OR⁹;
q is an integer from 0 to 3;
R⁴ is selected from halogen, hydroxy, optionally substituted C₁₋₆ alkyl, optionally substituted C₂₋₆ alkenyl, optionally substituted C₂₋₆ alkynyl, optionally substituted C₁₋₆ alkylthio, optionally substituted C₂₋₈ alkoxyalkyl, optionally substituted C₃₋₁₀ carbocyclyl, optionally substituted C₆₋₁₀ aryl, —CN, —OR⁹, —SR⁹, —NR⁹R¹⁰, —C(O)R⁹, —C(O)OR⁹, —S(O)₂R⁹, and —NO₂;
R⁵ is selected from H, optionally substituted C₁₋₆ alkyl, optionally substituted C₂₋₆ alkenyl, optionally substituted C₂₋₆ alkynyl, optionally substituted C₂₋₈ alkoxyalkyl, optionally substituted C₃₋₆ carbocyclyl, optionally substituted C₅₋₁₀ aryl, optionally substituted 5-6 membered heteroaryl, and optionally substituted 5-6 membered heterocyclyl;
L is a bond, or a linker selected from —C(O)N(R⁹)—, —S(O)₂—, —C(O)—, or —C(O)O—; or from optionally substituted C₁₋₄ alkylene, optionally substituted C₂₋₄ alkenylene, —OC₁₋₄ alkylene, or —OC₂₋₄ alkenylene, each of which is optionally interrupted with —O—, —S—, —NR⁹—, —NR¹⁰—, —C(O)N(R⁹)—, —N(R⁹) C(O)—, —S(O)₂—, —N(R⁹) S(O)₂—, —S(O)₂N(R⁹)—, —C(O)—, —C(O)O—, or —O(O)C—;
R⁶ is selected from C₅₋₁₀ aryl, 5-10 membered heteroaryl, 3-10 membered heterocyclyl, C₃₋₁₀ carbocyclyl, C₁₋₁₀ alkyl, C₂₋₁₀ alkenyl, or C₂₋₁₀ alkynyl, each of which is optionally substituted with one or more substituents optionally selected from optionally substituted C₁₋₆ alkyl, optionally substituted C₅₋₆ aryl, optionally substituted 5-6 membered heteroaryl, optionally substituted 5-6 membered heterocyclyl, halogen, —CN, —OR¹¹, —SR¹¹, —S(O)₂R¹¹, —S(O)₂N(R¹¹)₂, —S(O)(NR¹⁰)R¹¹, —C(O)N(R¹¹)₂, —NR¹¹C(O)R¹¹, —N(R¹¹) S(O)₂R¹¹, —N(R¹¹)₂, —NO₂,═(O), ═(S), ═(N)R¹¹, —C(O)R¹¹, —C(O)OR¹¹, —C(O)N(R¹¹)₂, or —C(O)SR¹¹; wherein two adjacent optional substituents, when present, together with the atoms to which they are attached, may form an optionally substituted fused 5-6 membered heteroaryl or an optionally substituted fused 5-6 membered heterocyclyl;
or R⁶ and R⁵, together with the atoms to which they are attached, form a 3-10 membered heterocyclyl or 5-10 membered heteroaryl, each of which is optionally substituted with one or more substituents optionally selected from optionally substituted C₁₋₁₀ alkyl, optionally substituted C₂₋₆ alkenyl, optionally substituted C₂₋₆ alkynyl, halogen, —CN, —OR¹¹, —SR¹¹, —S(O)₂R¹¹, —S(O)₂N(R¹¹)₂, —S(O)(NR¹⁰)R¹¹, —C(O) N(R¹¹)₂, —NR¹¹C(O)R¹¹, —N(R¹¹) S(O)₂R¹¹, —N(R¹¹)₂, —NO₂, ═(O), ═(S), ═(N)R¹¹, —C(O)R¹¹, —C(O)OR¹¹, —C(O)N(R¹¹)₂, or —C(O)SR¹¹;
each R¹¹ is independently selected from H, optionally substituted C₁₋₆ alkyl, optionally substituted C₂₋₆ alkenyl, optionally substituted C₂₋₆ alkynyl, optionally substituted C₅₋₁₀ aryl, optionally substituted C₂₋₂₀ alkoxyalkyl, C₁-C₆ haloalkyl, —C(O)R⁹, —OR⁹, —S(O)₂R⁹, and —S(O)₂N(R⁹) 2.
R⁷ and R⁸ are independently selected from H, C₁₋₆ alkyl, C₅₋₁₀ aryl, 5-10 membered heteroaryl, and C₃₋₁₀ carbocyclyl, each of which is optionally substituted with one or more substituents selected from C₁₋₆ alkyl, C₂₋₆ alkenyl, C₂₋₆ alkynyl, C₅₋₆ aryl, halogen, —CN, —OR⁹, —SR⁹, —S(O)₂R⁹, —NR⁹R¹⁰, —NO₂, —(O), ═(S), —(N)R⁹, ═(N)R¹⁰, —C(O)R⁹, —C(O)NR⁹R¹⁰, —C(O)SR⁹ or —C(O)OR⁹.

2. The compound according to claim 1, having the structure of Formula (III): 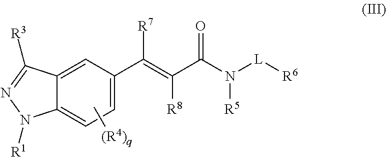
or a pharmaceutically acceptable salt, solvate or stereoisomer thereof.

3. The compound according to claim 1 or claim 2, having the structure of Formula (IV): 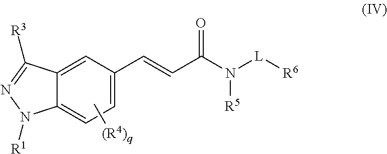
or a pharmaceutically acceptable salt, solvate or stereoisomer thereof.

4. The compound according to any one of claims 1 to 3, wherein R¹ is C₁₋₁₀ alkyl, C₂₋₁₀ alkenyl, C₂₋₁₀ alkynyl, or C₃₋₆ carbocyclyl, each of which is optionally substituted with one or more substituents selected from C₁₋₆ alkyl, halogen, CN, —OR⁹, and —SR⁹; or with one or more groups selected from C₃₋₆ carbocyclyl, 5-6 membered heterocyclyl, C₅₋₆ aryl, and 5-6 membered heteroaryl, each of which is optionally substituted with one or more substituents selected from C₁₋₆ alkyl, halogen, —CN, —OR⁹, —SR⁹, —S(O)₂R⁹, —NR⁹R¹⁰, —NO₂, —(O), ═(S), ═(N)R⁹, ═(N)R¹⁰, —C(O)R⁹, —C(O)NR⁹R¹⁰, —C(O)SR⁹ or —C(O)OR⁹.

5. The compound according to any one of claims 1 to 4, wherein R¹ is C₁₋₆ alkyl, optionally substituted with one or more substituents selected from C₁₋₆ alkyl, halogen, —CN, —OR⁹, and —SR⁹; or with one or more groups selected from C₃₋₆ carbocyclyl, 5-6 membered heterocyclyl, C₅₋₆ aryl, and 5-6 membered heteroaryl, each of which is optionally substituted with one or more substituents selected from C₁₋₆ alkyl, halogen, —CN, —OR⁹, —SR⁹, —S(O)₂R⁹, —NR⁹R¹⁰, —NO₂, ═(O), ═(S), ═(N)R⁹, —(N)R¹⁰, —C(O)R⁹, —C(O) NR⁹R¹⁰, —C(O)SR⁹ or —C(O)OR⁹.

6. The compound according to any one of claims 1 to 5, wherein R¹ is C₁₋₆ alkyl, optionally selected from methyl, ethyl, propyl or isopropyl.

7. The compound according to any one of claims 1 to 5, wherein R¹ is C₁₋₆ alkyl substituted with halogen, —CN, —OR⁹, —C(O)R⁹, —C(O)NR⁹R¹⁰, C(O)SR⁹ or —C(O)OR⁹, optionally wherein R⁹ is methyl.

8. The compound according to any one of claims 1 to 5, wherein R¹ is C₁₋₆ alkyl substituted with optionally substituted 5-6 membered heteroaryl or optionally substituted 3-6 membered heterocyclyl.

9. The compound according to claim 8, wherein R¹ is C₁₋₄ alkyl substituted with a 3-6 membered heterocyclyl selected from: 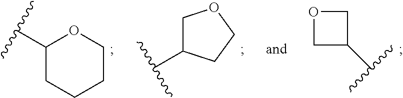 [SMILES: *C1CCCCO1 ; *C1CCOC1 ; *C1COC1]
each of which may be optionally substituted.

10. The compound according to any one of claims 1 to 5, wherein R¹ is C₁₋₆ alkyl substituted with optionally substituted C₁₋₆ carbocyclyl.

11. The compound according to claim 10, wherein R¹ is C₁₋₄ alkyl substituted with C₁₋₆ carbocyclyl, optionally wherein C₁₋₆ carbocyclyl is selected from optionally substituted cyclobutyl or optionally substituted cyclopropyl.

12. The compound according to claim 9 or claim 10, wherein R¹ is

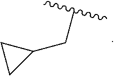 [SMILES: *CC1CC1]

13. The compound according to any one of claims 1 to 12, wherein R³ is H.

14. The compound according to any one of claims 1 to 13, wherein q is 0 or 1.

15. The compound according to any one of claims 1 to 14, having the structure of Formula (Va), (Vb) or (Vc): 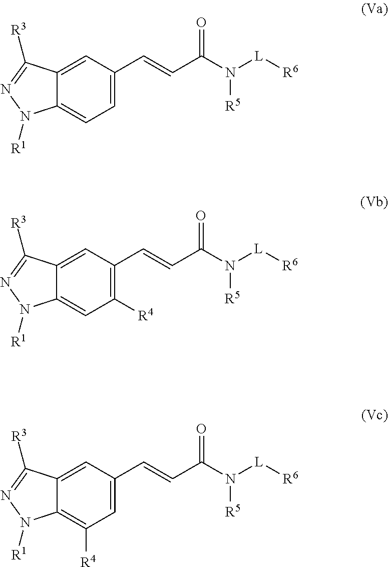
or a pharmaceutically acceptable salt, solvate or stereoisomer thereof.

16. The compound according to any one of claims 1 to 15, wherein R⁴ is selected from halogen, hydroxy, optionally substituted C₂₋₈ alkoxyalkyl, —CN, —OR⁹, —SR⁹, —NR⁹R¹⁰, —C(O)R⁹, —C(O)OR⁹, —S(O)₂R⁹, and —NO₂.

17. The compound according to any one of claims 1 to 16, wherein R⁴ is selected from halogen, —CN, and —NO₂, optionally wherein halogen is F.

18. The compound according to any one of claims 1 to 17, having the structure of Formula (VI): 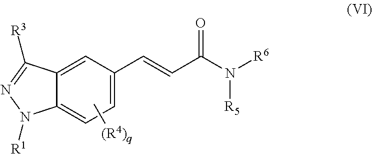
or a pharmaceutically acceptable salt, solvate or stereoisomer thereof.

19. The compound according to any one of claims 1 to 18, having the structure of Formula (VIa), (VIb) or (VIc): 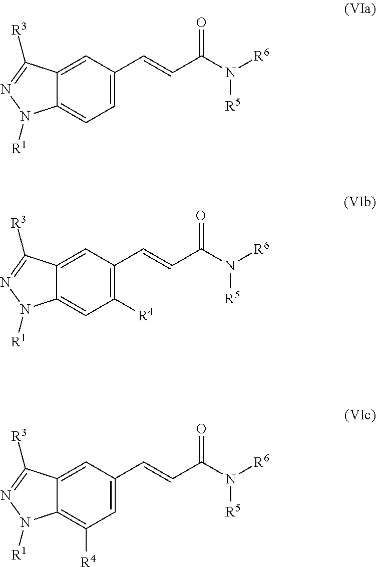
or a pharmaceutically acceptable salt, solvate or stereoisomer thereof.

20. The compound according to any one of claims 1 to 19, wherein R⁵ is H, optionally substituted C₁₋₄ alkyl, optionally substituted C₂₋₄ alkenyl, optionally substituted C₂₋₄ alkynyl, or optionally substituted C₂₋₈ alkoxyalkyl.

21. The compound according to any one of claims 1 to 20, wherein R⁵ is H.

22. The compound according to any one of claims 1 to 21, wherein R⁶ is selected from C₅₋₁₀ aryl, 5-10 membered heteroaryl, 3-10 membered heterocyclyl, C3-10 carbocyclyl, C₁₋₁₀ alkyl, C₂₋₁₀ alkenyl, or C₂₋₁₀ alkynyl, each of which is optionally substituted with one or more substituents optionally selected from optionally substituted C₁₋₆ alkyl, optionally substituted C₅₋₆ aryl, optionally substituted 5-6 membered heteroaryl, optionally substituted 5-6 membered heterocyclyl, halogen, —CN, —OR¹¹, —SR¹¹, —S(O)₂R¹¹, —S(O)₂N(R¹¹)₂, —S(O)(NR¹⁰)R¹¹, —C(O)N(R¹¹)₂, —NR¹¹C(O)R¹¹, —N(R¹¹) S(O)₂R¹¹, —N(R¹¹)₂, —NO₂,═(O),═(S), ═(N)R¹¹, —C(O)R¹¹, —C(O)OR¹¹, —C(O)N(R¹¹)₂, or —C(O)SR¹¹; wherein two adjacent optional substituents, when present, together with the atoms to which they are attached, may form an optionally substituted fused 5-6 membered heteroaryl or an optionally substituted fused 5-6 membered heterocyclyl.

23. The compound according to any one of claims 1 to 22, wherein R⁶ is selected from optionally substituted C6 aryl, optionally substituted 5 membered heteroaryl, and optionally substituted 6 membered heteroaryl.

24. The compound according to any one of claims 1 to 23, wherein R⁶ is selected from: 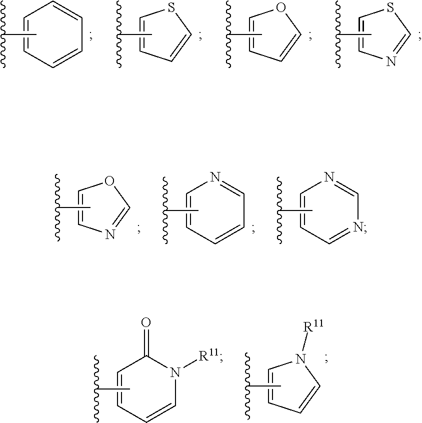
each of which may be optionally substituted.

25. The compound according to any one of claims 1 to 24, wherein R⁶ is selected from: 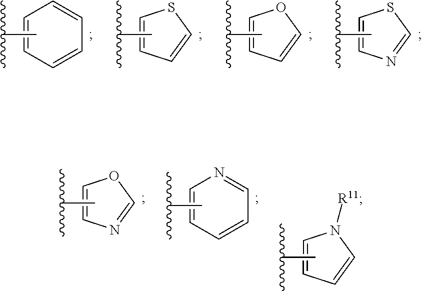
each of which may be optionally substituted.

26. The compound according to any one of claims 1 to 25, wherein the optional substituent of R⁶ is one or more substituents selected from halogen, optionally substituted 5 membered heterocyclyl, optionally substituted 5 membered heteroaryl, —C(O)OR¹¹, —N(R¹¹) S(O)₂R¹¹, —C(O)N(R¹¹)₂, and —S(O)₂N(R¹¹)₂.

27. The compound according to any one of claims 1 to 26, selected from the group consisting of: 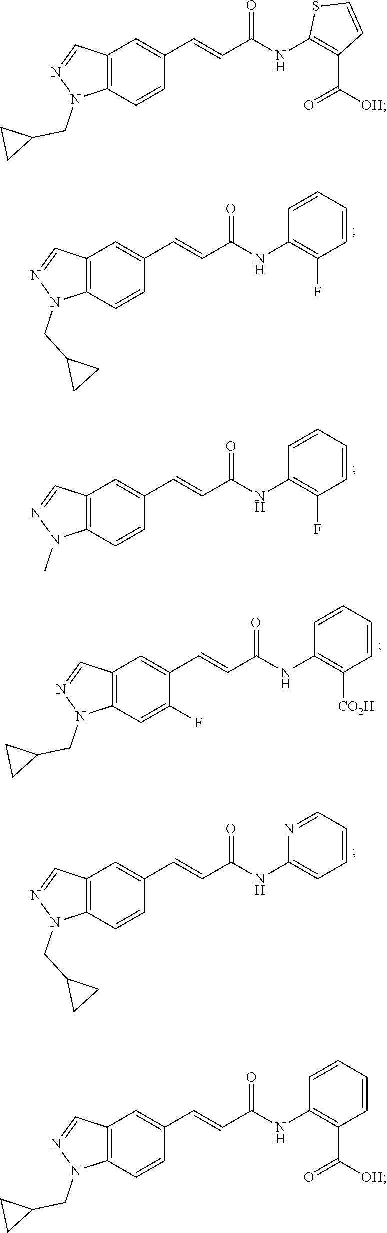 [SMILES: O=C(/C=C/c1ccc2c(cnn2CC2CC2)c1)Nc1sccc1C(=O)O ; O=C(/C=C/c1ccc2c(cnn2CC2CC2)c1)Nc1ccccc1F ; Cn1ncc2cc(/C=C/C(=O)Nc3ccccc3F)ccc21 ; O=C(/C=C/c1cc2cnn(CC3CC3)c2cc1F)Nc1ccccc1C(=O)O ; O=C(/C=C/c1ccc2c(cnn2CC2CC2)c1)Nc1ccccn1 ; O=C(/C=C/c1ccc2c(cnn2CC2CC2)c1)Nc1ccccc1C(=O)O] 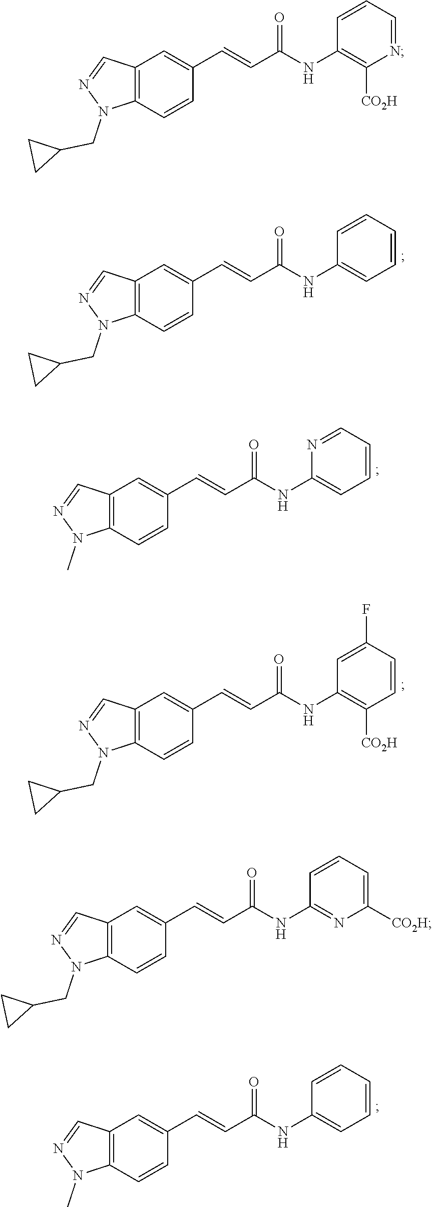 [SMILES: O=C(/C=C/c1ccc2c(cnn2CC2CC2)c1)Nc1cccnc1C(=O)O ; O=C(/C=C/c1ccc2c(cnn2CC2CC2)c1)Nc1ccccc1 ; Cn1ncc2cc(/C=C/C(=O)Nc3ccccn3)ccc21 ; O=C(/C=C/c1ccc2c(cnn2CC2CC2)c1)Nc1cc(F)ccc1C(=O)O ; O=C(/C=C/c1ccc2c(cnn2CC2CC2)c1)Nc1cccc(C(=O)O)n1 ; Cn1ncc2cc(/C=C/C(=O)Nc3ccccc3)ccc21] 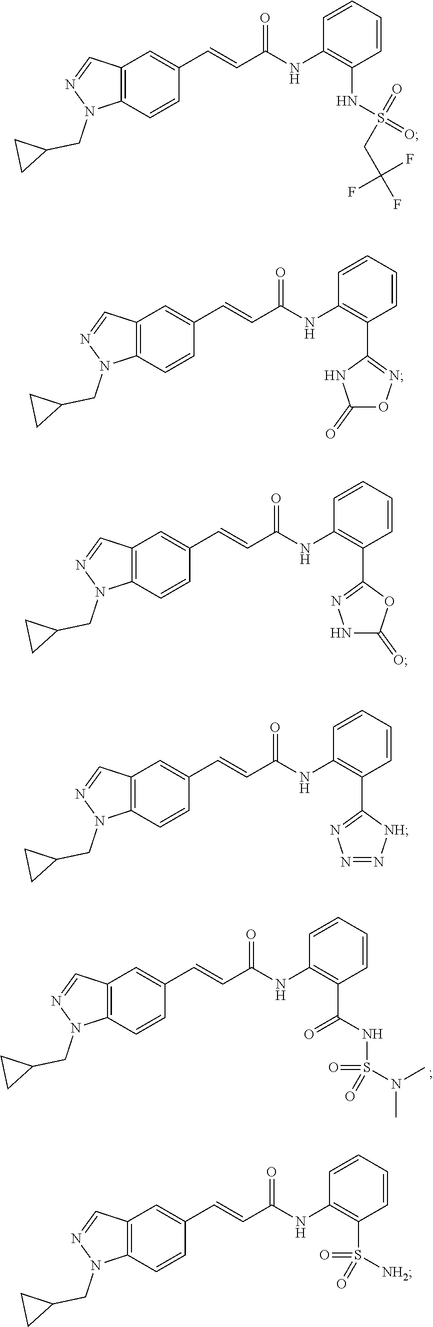 [SMILES: O=C(/C=C/c1ccc2c(cnn2CC2CC2)c1)Nc1ccccc1NS(=O)(=O)CC(F)(F)F ; O=C(/C=C/c1ccc2c(cnn2CC2CC2)c1)Nc1ccccc1-c1noc(=O)[nH]1 ; O=C(/C=C/c1ccc2c(cnn2CC2CC2)c1)Nc1ccccc1-c1n[nH]c(=O)o1 ; O=C(/C=C/c1ccc2c(cnn2CC2CC2)c1)Nc1ccccc1-c1nnn[nH]1 ; CN(C)S(=O)(=O)NC(=O)c1ccccc1NC(=O)/C=C/c1ccc2c(cnn2CC2CC2)c1 ; NS(=O)(=O)c1ccccc1NC(=O)/C=C/c1ccc2c(cnn2CC2CC2)c1] 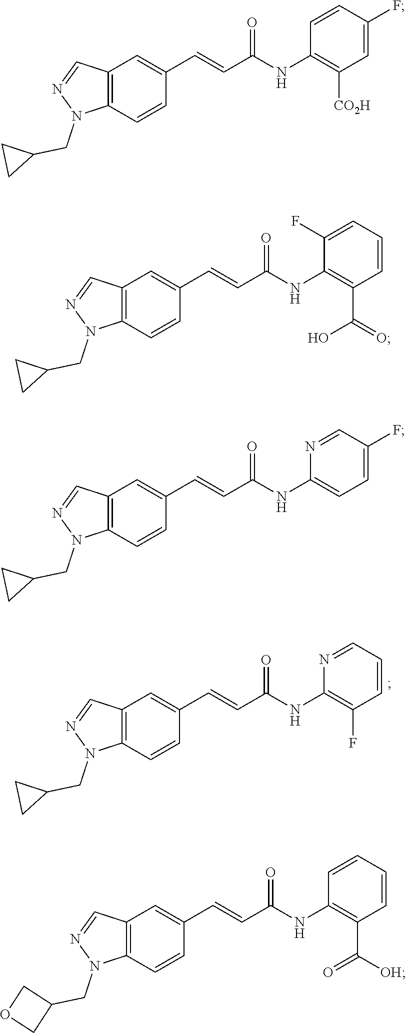 [SMILES: O=C(/C=C/c1ccc2c(cnn2CC2CC2)c1)Nc1ccc(F)cc1C(=O)O ; O=C(/C=C/c1ccc2c(cnn2CC2CC2)c1)Nc1c(F)cccc1C(=O)O ; O=C(/C=C/c1ccc2c(cnn2CC2CC2)c1)Nc1ccc(F)cn1 ; O=C(/C=C/c1ccc2c(cnn2CC2CC2)c1)Nc1ncccc1F ; O=C(/C=C/c1ccc2c(cnn2CC2COC2)c1)Nc1ccccc1C(=O)O]
or a pharmaceutically acceptable salt, solvate or stereoisomer thereof.

28. The compound according to any one of claims 1 to 27, selected from the group consisting of: 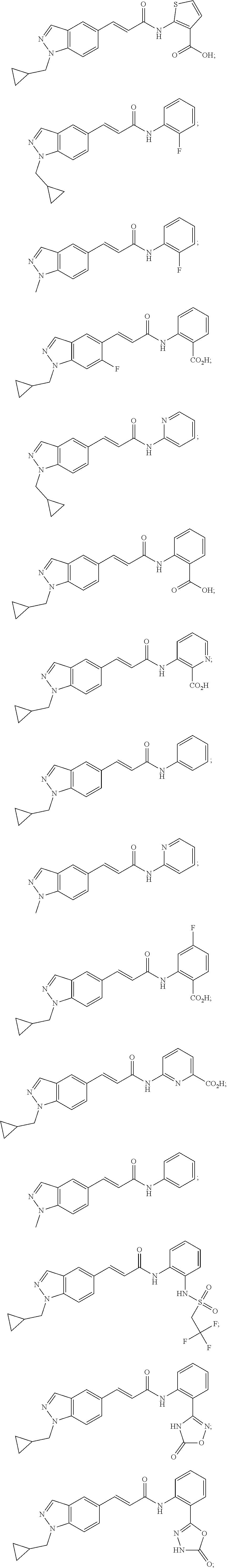
or a pharmaceutically acceptable salt, solvate or stereoisomer thereof.

29. The compound according to any one of claims 1 to 28, selected from the group consisting of: 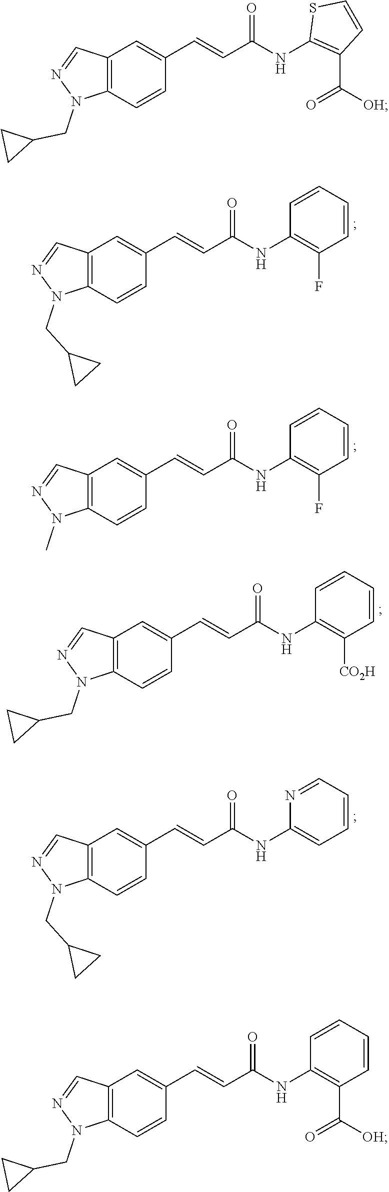 [SMILES: O=C(/C=C/c1ccc2c(cnn2CC2CC2)c1)Nc1sccc1C(=O)O ; O=C(/C=C/c1ccc2c(cnn2CC2CC2)c1)Nc1ccccc1F ; Cn1ncc2cc(/C=C/C(=O)Nc3ccccc3F)ccc21 ; O=C(/C=C/c1ccc2c(cnn2CC2CC2)c1)Nc1ccccc1C(=O)O ; O=C(/C=C/c1ccc2c(cnn2CC2CC2)c1)Nc1ccccn1 ; O=C(/C=C/c1ccc2c(cnn2CC2CC2)c1)Nc1ccccc1C(=O)O]
or a pharmaceutically acceptable salt, solvate or stereoisomer thereof.

30. The compound according to any one of claims 1 to 22, wherein R⁶ is selected from optionally substituted 3-10 membered heterocyclyl and optionally substituted C₃₋₁₀ carbocyclyl.

31. The compound according to claim 30, wherein the optional substituent of R⁶ is one or more substituents selected from halogen and —C(O)OR¹¹, optionally wherein R¹¹ is H or optionally substituted C₁₋₆ alkyl.

32. The compound according to claim 30 or claim 31, selected from the group consisting of: 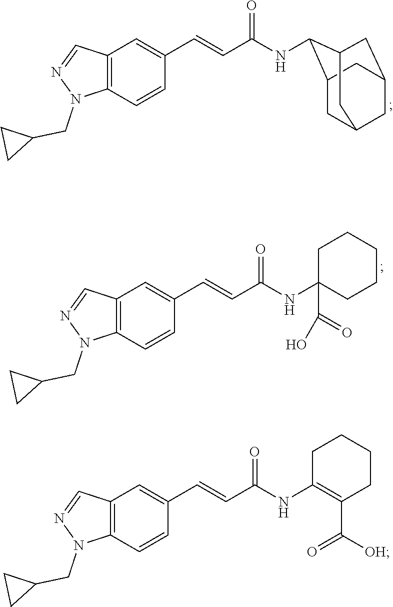 [SMILES: O=C(/C=C/c1ccc2c(cnn2CC2CC2)c1)NC1C2CC3CC(C2)CC1C3 ; O=C(/C=C/c1ccc2c(cnn2CC2CC2)c1)NC1(C(=O)O)CCCCC1 ; O=C(/C=C/c1ccc2c(cnn2CC2CC2)c1)NC1=C(C(=O)O)CCCC1]
or a pharmaceutically acceptable salt, solvate or stereoisomer thereof.

33. The compound according to any one of claims 1 to 22, wherein R⁶ and R⁵, together with the atoms to which they are attached, form a 3-10 membered heterocyclyl or 5-10 membered heteroaryl, each of which is optionally substituted with one or more substituents optionally selected from optionally substituted C₁₋₁₀ alkyl, optionally substituted C₂₋₆ alkenyl, optionally substituted C₂₋₆ alkynyl, halogen, —CN, —OR¹¹, —SR¹¹, —S(O)₂R¹¹, —S(O)₂N(R¹¹)₂, —S(O)(NR¹⁰)R¹¹, —C(O)N(R¹¹)₂, —NR¹¹C(O)R¹¹, —N(R¹¹) S(O)₂R¹¹, —N(R¹¹)₂, —NO₂,═(O),═(S), ═(N)R¹¹, —C(O)R¹¹, —C(O)OR¹¹, —C(O) N(R¹¹)₂, or —C(O)SR¹¹.

34. The compound according to claim 33, wherein the optional substituent of R⁶ is one or more substituents selected from halogen and —C(O)OR¹¹, optionally wherein R¹¹ is H or optionally substituted C₁₋₆ alkyl.

35. The compound according to claim 33 or claim 34, having the structure: 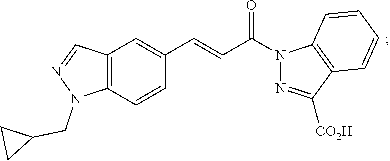 [SMILES: O=C(O)c1nn(C(=O)/C=C/c2ccc3c(cnn3CC3CC3)c2)c2ccccc12]
or a pharmaceutically acceptable salt, solvate or stereoisomer thereof.

36. A compound having the structure: 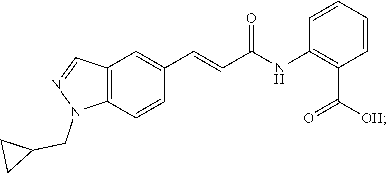 [SMILES: O=C(/C=C/c1ccc2c(cnn2CC2CC2)c1)Nc1ccccc1C(=O)O]
or a pharmaceutically acceptable salt thereof.

37. The compound or pharmaceutically acceptable salt thereof according to claim 36, wherein the compound is:

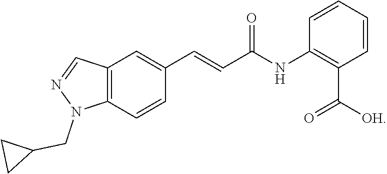 [SMILES: O=C(/C=C/c1ccc2c(cnn2CC2CC2)c1)Nc1ccccc1C(=O)O]

38. A pharmaceutical composition comprising a compound according to any one of claims 1 to 37, or a pharmaceutically acceptable salt, solvate or stereoisomer thereof, and a pharmaceutically acceptable excipient.

39. A method of treating and/or preventing a disease, disorder or condition mediated by a proton-activated GPCR in a subject in need thereof, comprising administering to the subject an effective amount of a compound according to any one of claims 1 to 37, or a pharmaceutically acceptable salt, solvate or stereoisomer thereof, or a pharmaceutical composition according to claim 38.

40. Use of a compound according to any one of claims 1 to 37, or a pharmaceutically acceptable salt, solvate or stereoisomer thereof, or a pharmaceutical composition according to claim 38, in the manufacture of a medicament for the treatment and/or prevention of a disease, disorder or condition mediated by a proton-activated GPCR in a subject in need thereof.

41. A compound according to any one of claims 1 to 37, or a pharmaceutically acceptable salt, solvate or stereoisomer thereof, or a pharmaceutical composition according to claim 38, for use in the treatment and/or prevention of a disease, disorder or condition mediated by a proton-activated GPCR in a subject in need thereof.

42. The method according to claim 39, the use according to claim 40, or the compound or a pharmaceutically acceptable salt, solvate or stereoisomer thereof or the pharmaceutical composition for use according to claim 41, wherein the proton-activated GPCR is a GPR68 (OGR1).

43. The method, use, or the compound or a pharmaceutically acceptable salt, solvate or stereoisomer thereof or the pharmaceutical composition for use according to any one of claims 39 to 42, wherein the disease, disorder or condition is selected from the group consisting of: an inflammatory disease, disorder or condition; a fibrotic disease, disorder or condition; a proliferative disease, disorder or condition, or a cancer.

44. The method, use, or the compound or a pharmaceutically acceptable salt, solvate or stereoisomer thereof or the pharmaceutical composition for use according to claim 43, wherein the disease, disorder or condition is an inflammatory disease, disorder or condition.

45. The method, use, or the compound or a pharmaceutically acceptable salt, solvate or stereoisomer thereof or the pharmaceutical composition for use according to claim 44, wherein the inflammatory disease, disorder or condition is selected from focal segmental glomerulosclerosis, lupus nephritis, membranous nephropathy, IgA nephropathy, chronic obstructive pulmonary disorder (COPD), asthma, and cystic fibrosis.

46. The method, use, or the compound or a pharmaceutically acceptable salt, solvate or stereoisomer thereof or the pharmaceutical composition for use according to claim 44, wherein the inflammatory disease, disorder or condition is an autoimmune disease, disorder or condition.

47. The method, use, or the compound or a pharmaceutically acceptable salt, solvate or stereoisomer thereof or the pharmaceutical composition for use according to claim 46, wherein the autoimmune disease, disorder or condition is selected from systemic lupus erythematosus (SLE), inflammatory bowel disease, Crohn's disease, ulcerative colitis, graft versus host disease, multiple sclerosis, and rheumatoid arthritis.

48. The method, use, or the compound or a pharmaceutically acceptable salt, solvate or stereoisomer thereof or the pharmaceutical composition for use according to claim 43, wherein the disease, disorder or condition is a fibrotic disease, disorder or condition.

49. The method, use, or the compound or a pharmaceutically acceptable salt, solvate or stereoisomer thereof or the pharmaceutical composition for use according to claim 48, wherein the fibrotic disease, disorder or condition is selected from renal fibrosis, pulmonary fibrosis, cardiovascular fibrosis, ocular fibrosis, dermal fibrosis, pancreatic fibrosis, and liver fibrosis.

50. The method, use, or the compound or a pharmaceutically acceptable salt, solvate or stereoisomer thereof or the pharmaceutical composition for use according to claim 48, wherein the fibrotic disease, disorder or condition is selected from diabetic cardiomyopathy, congestive heart failure, ischemic heart disease, hypertension, peripheral artery disease, cerebrovascular disease, chronic kidney disease, diabetic nephropathy, systemic sclerosis (scleroderma), hypertrophic scars, keloids, idiopathic pulmonary fibrosis (IPF), non-alcoholic steatohepatitis (NASH), non-alcoholic fatty liver disease (NAFLD), primary biliary cirrhosis (PBC), and primary sclerosis cholangitis (PSC).

51. The method, use, or the compound or a pharmaceutically acceptable salt, solvate or stereoisomer thereof or the pharmaceutical composition for use according to claim 43, wherein the disease, disorder or condition is a proliferative disease, disorder or condition.

52. The method, use, or the compound or a pharmaceutically acceptable salt, solvate or stereoisomer thereof or the pharmaceutical composition for use according to claim 51, wherein the proliferative disease, disorder or condition is selected from diabetic retinopathy, diabetic macular edema, macular degeneration, retinopathy of prematurity (ROP), and proliferative vitreoretinopathy (PVR).

53. The method, use, or the compound or a pharmaceutically acceptable salt, solvate or stereoisomer thereof or the pharmaceutical composition for use according to claim 43, wherein the disease, disorder or condition is a cancer.

54. The method, use, or the compound or a pharmaceutically acceptable salt, solvate or stereoisomer thereof or the pharmaceutical composition for use according to claim 53, wherein the cancer is selected from a breast cancer, skin cancer (melanoma), pancreatic cancer, lung cancer, prostate cancer, colon cancer, liver cancer (hepatocellular carcinoma), head and neck cancer, ovarian cancer and kidney cancer.

55. The method, use, or the compound or a pharmaceutically acceptable salt, solvate or stereoisomer thereof or the pharmaceutical composition for use according to any one of claims 49 to54, wherein the subject is a human.

56. A method of inhibiting a proton-activated GPCR in a cell, comprising administering to the cell an effective amount of a compound according to any one of claims 1 to 37, or a pharmaceutically acceptable salt, solvate or stereoisomer thereof, or a pharmaceutical composition according to claim 38.

57. Use of a compound according to any one of claims 1 to 37, or a pharmaceutically acceptable salt, solvate or stereoisomer thereof, or a pharmaceutical composition according to claim 38, in the manufacture of a medicament for the inhibition of a proton-activated GPCR in a cell.

58. A compound according to any one of claims 1 to 37, or a pharmaceutically acceptable salt, solvate or stereoisomer thereof, or a pharmaceutical composition according to claim 38, for use in the inhibition of a proton-activated GPCR in a cell.

59. The method according to claim 56, the use according to claim 57, or the compound or a pharmaceutically acceptable salt, solvate or stereoisomer thereof or the pharmaceutical composition for use according to claim 58, wherein the proton-activated GPCR is a GPR68 (OGR1).

60. The method, use, or the compound or a pharmaceutically acceptable salt, solvate or stereoisomer thereof or the pharmaceutical composition for use according to any one of claims 56 to 59, wherein the cell is a tumour cell.
